(19) United States
(12) Patent Application Publication
(10) Pub. No.: US 2021/0388389 A1
(43) Pub. Date: Dec. 16, 2021

(54) COMPOSITIONS AND METHODS FOR RAPID AND MODULAR GENERATION OF CHIMERIC ANTIGEN RECEPTOR T CELLS

(71) Applicant: Yale University, New Haven, CT (US)

(72) Inventors: Sidi Chen, Milford, CT (US); Xiaoyun Dai, West Haven, CT (US)

(21) Appl. No.: 17/290,641

(22) PCT Filed: Oct. 22, 2019
(86) PCT No.: PCT/US2019/057379

§ 371 Date: Apr. 30, 2021

Related U.S. Application Data
(60) Provisional application No. 62/752,684, filed on Oct. 30, 2018.
(60) Provisional application No. 62/790,622, filed on Jan. 10, 2019.

(51) Int. Cl. C12N 15/86; C12N 5/0783

(52) U.S. Cl. CPC C12N 15/86; C12N 2501/70; C12N 2501/515; C12N 5/0636

(57) ABSTRACT

Disclosed are compositions and methods for cellular genome engineering that permit simple, efficient, and versatile permutations of combinatorial or simultaneous knockout and knock-in genomic modifications. An exemplary method includes modifying the genome of a cell by introducing to the cell a Cpf1 endonuclease and one or more AAV vectors encoding one or more crRNAs that direct the endonuclease to one or more target genes. The AAV vectors further contain one or more HDR templates that provide a sequence that encodes a reporter gene, a chimeric antigen receptor (CAR), or combinations thereof, and sequences homologous to one or more target sites. Also disclosed are pharmaceutical compositions containing genetically modified cells and methods of use thereof in treating a subject having a disease or disorder, such as cancer. The disclosed compositions and methods are especially applicable to development of enhanced chimeric antigen receptor engineered T cell therapy (CAR-T).

### CROSS-REFERENCE TO RELATED APPLICATIONS

This application claims the benefit of and priority to U.S. Provisional Application No. 62/752,684 filed Oct. 30, 2018, and U.S. Provisional Application No. 62/790,622 filed Jan. 10, 2019, which are hereby incorporated by reference in their entirety.

### STATEMENT REGARDING FEDERALLY SPONSORED RESEARCH

This invention was made with Government Support under CA238295 and CA209992 awarded by the National Institutes of Health (NIH). The Government has certain rights in the invention.

### REFERENCE TO SEQUENCE LISTING

The Sequence Listing submitted Oct. 22, 2019 as a text file named “YU_7536_PCT_ST25.txt,” created on Oct. 16, 2019, and having a size of 8,069 bytes is hereby incorporated by reference pursuant to 37 C.F.R. § 1.52(e)(5).

### FIELD OF THE INVENTION

The invention is generally related to the fields of gene editing technology and immunotherapy, and more particularly to improved methods of engineering enhanced chimeric antigen receptor T cells using Cpf1 and AAV mediated delivery of crRNAs/HDR templates.

### BACKGROUND OF THE INVENTION

Adoptive immunotherapy, in which T cells that are specific for tumor-associated antigens are expanded to generate large numbers of cells and transferred into tumor-bearing hosts, is a promising strategy to treat cancer. The T cells used for adoptive immunotherapy can be generated either by expansion of antigen-specific T cells or redirection of T cells through genetic engineering. One approach to genetically engineering T cells is to modify the cells to target antigens expressed on tumor cells through the expression of chimeric antigen receptors (CARs). CARs are antigen receptors that are designed to recognize cell surface antigens in a human leukocyte antigen-independent manner Upon recognition and binding of the antigen, the CAR T cell activates an immune response against the antigen bearing cells.

Engineered CAR T cell treatments of patients with cancer have shown promising clinical results. For example, genetically modified T cells expressing anti-CD19 CARs have recently been approved for the treatment of patients with relapsed or refractory diffuse large B-cell lymphoma and B-cell acute lymphoblastic leukemia. However, the majority of current CAR T clinical trials utilize autologous T cells, which are often limited by poor quality and quantity of T cells, as well as the time and expense of manufacturing autologous T cell products. These limitations could be circumvented by the use of allogeneic CAR T cells, further modified to reduce risks of graft-versus-host disease (where the endogenous T cell receptor (TCR) on allogeneic T cells recognize the alloantigens of the recipient) and rejection by the host immune system (e.g., human leukocyte antigen (HLA) on the surface of allogeneic T cells causes rejection by the host). Such modifications encompass both individual and dual disruption of endogenous TCR and HLA class I genes to generate ‘universal’ CAR T cells.

In the context of solid cancers, the efficacy to date of CAR T cell therapy has been variable due to tumor-evolved mechanisms that inhibit local immune cell activity. To bolster the potency of CAR-T cells, modulation of the immunosuppressive tumor microenvironment with immune-checkpoint blockade is a promising strategy. It is therefore desirable to reduce immunosuppression of CAR T cell activity, e.g., through inactivation of immune checkpoint proteins. Therefore, simple and efficient methods are needed for multiplex genomic editing of T cells.

Another impediment to the clinical application of CAR T technology to date has been limited in vivo expansion of CAR+ T cells, rapid disappearance of the cells after infusion, and disappointing clinical activity (Jena, et al., Blood., 116:1035-1044 (2010); Uckun, et al., Blood., 71: 13-29 (1988)). Current T cell transgene delivery methods in the clinic are based on randomly integrating lentiviral and γ-retroviral vectors, which carry the risk of insertional oncogenesis and translational silencing. Continuous CAR transgene expression in primary T cells by these viral vectors requires repeated transduction, and further, infusion of new cells for sustained expression in patients. Moreover, multiplex gene editing in CAR-T cells, though currently possible with Cas9 nuclease, requires lentivirus transduction followed by electroporation of multiple components including Cas9 and guide RNAs, which complicates a manufacturing process that must adhere to Current Good Manufacturing Practice (cGMP) regulations.

Thus, there is an urgent need of alternative approaches for generation of CAR T that simplify the manufacturing process, as well as for improved CAR T therapy that shows reduced risk of immune rejection, reduced exhaustion, and enhanced stability and effector function.

Therefore, it is an object of the invention to provide enhanced methods of CAR T cell generation that simplify the cGMP manufacturing process.

It is another object of the invention to provide CAR T cells that exhibit more stable CAR transgene expression.

It is yet another object of the invention to provide CAR T cells that exhibit increased cytotoxic activity, higher levels of effector cytokine production, and lower levels of exhaustion markers.

It is a further object of the invention to provide more efficient methods of achieving multiplexed genomic modifications including combinatorial targeted gene knockouts and targeted knock-ins (e.g., non-random transgene integrations).

Any discussion of documents, acts, materials, devices, articles or the like which has been included in the present specification is not to be taken as an admission that any or all of these matters form part of the prior art base or were common general knowledge in the field relevant to the present disclosure as it existed before the priority date of each claim of this application.

Throughout this specification the word “comprise,” or variations such as “comprises” or “comprising,” will be understood to imply the inclusion of a stated element, integer or step, or group of elements, integers or steps, but not the exclusion of any other element, integer or step, or group of elements, integers or steps.

### SUMMARY OF THE INVENTION

Compositions and methods for cellular genomic engineering (e.g., T cell engineering) that permit simple, efficient, and versatile combinations of multiplexed knockout and knock-in genomic modifications are provided. The disclosed compositions and methods are especially applicable to development of enhanced chimeric antigen receptor engineered T cell therapy (CAR-T).

An exemplary method includes modifying the genome of a cell by introducing to the cell an RNA-guided endonuclease and one or more AAV vectors containing a sequence (e.g., a crRNA array) that encodes one or more crRNAs that collectively direct the endonuclease to one or more target genes. Optionally, at least one of the AAV vectors contains or further contains one or more HDR templates. The crRNA array can encode two or more crRNAs each of which direct the endonuclease to a different target gene. In some forms, the method can involve introducing two AAV vectors. In the foregoing method, the one or more HDR templates include (a) a sequence that encodes a reporter gene, a chimeric antigen receptor (CAR), or combinations thereof; and (b) one or more sequences homologous to one or more target sites. The HDR template can further include a promoter and/or polyadenylation signal operationally linked to each reporter gene, CAR, or combination thereof.

The RNA-guided endonuclease can cause disruption of the target genes and/or the one or more HDR templates can mediate targeted integration of the reporter gene, the CAR, or combinations thereof at the target sites. A target site can be within the locus of the disrupted gene or at a locus different from the disrupted gene. Exemplary target genes or target sites include, but are not limited to PDCD1, TRAC, CTLA4, B2M, TRBC1, and TRBC2. Other non-limiting examples of target genes or target sites include those provided in Table 2. In some forms, the PDCD1 and/or TRAC gene can be disrupted; one or more reporter genes, one or more CARs, or combinations thereof can be integrated in the PDCD1 and/or TRAC gene; the PDCD1 gene can be disrupted and the one or more reporter genes, one or more CARs, or combinations thereof can be integrated in the TRAC gene; or the TRAC gene can disrupted and the one or more reporter genes, one or more CARs, or combinations thereof can be integrated in the PDCD1 gene.

The CAR can target one or more antigens specific for cancer, an inflammatory disease, a neuronal disorder, HIV/AIDS, diabetes, a cardiovascular disease, an infectious disease, an autoimmune disease, or combinations thereof. Exemplary antigens include, but are not limited to, antigens listed in Table 3 such as AFP, AKAP-4, ALK, Androgen receptor, B7H3, BCMA, Bcr-Abl, BORIS, Carbonic, CD123, CD138, CD174, CD19, CD20, CD22, CD30, CD33, CD38, CD80, CD86, CEA, CEACAMS, CEACAM6, Cyclin, CYP1B1, EBV, EGFR, EGFR806, EGFRvIII, EpCAM, EphA2, ERG, ETV6-AML, FAP, Fos-related antigenl, Fucosyl, fusion, GD2, GD3, GloboH, GM3, gp100, GPC3, HER-2/neu, HER2, HMWMAA, HPV E6/E7, hTERT, Idiotype, IL12, IL13RA2, IM19, IX, LCK, Legumain, IgK, LMP2, MAD-CT-1, MAD-CT-2, MAGE, MelanA/MART1, Mesothelin, MET, ML-IAP, MUC1, Mutant p53, MYCN, NA17, NKG2D-L, NY-BR-1, NY-ESO-1, NY-ESO-1, OY-TES1, p53, Page4, PAP, PAX3, PAXS, PD-L1, PDGFR-β, PLAC1, Polysialic acid, Proteinase3 (PR1), PSA, PSCA, PSMA, Ras mutant, RGSS, RhoC, ROR1, SART3, sLe(a), Sperm protein 17, SSX2, STn, Survivin, Tie2, Tn, TRP-2, Tyrosinase, VEGFR2, WT1, and XAGE. The CAR can be an anti-CD19 CAR (e.g., CD19BBz) or an anti-CD22 CAR (CD22BBz). In some forms, the CAR can be bispecific or multivalent.

The RNA-guided endonuclease can be introduced to the cell via an mRNA that encodes the RNA-guided endonuclease. The mRNA can contain modifications such as N6-methyladenosine (m6A), 5-methylcytosine (m5C), pseudouridine (w), N1-methylpseudouridine (me1ψ), and 5-methoxyuridine (5moU); a 5′ cap; a poly(A) tail; one or more nuclear localization signals; or combinations thereof.

The mRNA can be codon optimized for expression in a eukaryotic cell and can be introduced to the cell via electroporation, transfection, and/or nanoparticle mediated delivery. The RNA-guided endonuclease can also be introduced via a viral vector that encodes the RNA-guided endonuclease, or direct electroporation of the endonuclease protein or endonuclease protein-RNA complex.

A preferred RNA-guided endonuclease is Cpf1, or a variant, derivative, or fragment thereof, such as, for example, Cpf1 derived from Francisella novicida U112 (FnCpf1), Acidaminococcus sp. BV3L6 (AsCpf1), Lachnospiraceae bacterium ND2006 (LbCpf1), Lachnospiraceae bacterium MA2020 (Lb2Cpf1), Lachnospiraceae bacterium MC2017 (Lb3Cpf1), Moraxella bovoculi 237 (MbCpf1), Butyrivibrio proteoclasticus (BpCpf1), Parcubacteria bacterium GWC2011_GWC2_44_17 (PbCpf1); Peregrinibacteria bacterium GW2011_GWA_33_10 (PeCpf1), Leptospira inadai (LiCpf1), Smithella sp. SC_K08D17 (SsCpf1), Porphyromonas crevioricanis (PcCpf1), Porphyromonas macacae (PmCpf1), Candidatus Methanoplasma termitum (CMtCpf1), Eubacterium eligens (EeCpf1), Moraxella bovoculi 237 (MbCpf1), or Prevotella disiens (PdCpf1). In some preferred forms, the RNA guided endonuclease can be a Cpf1 ortholog, variant, or engineered derivative, derived from the bacterial species listed in Table 1. In some forms, the Cpf1 is a wildtype protein, a humanized Cpf1, a variant, a derivative, a fragment, a shuffled domain version, or combinations thereof. In some forms, the Cpf1 is LbCpf1, or a variant, derivative, or fragment thereof.

The AAV vector used in the disclosed compositions and methods can be a naturally occurring serotype of AAV including, but not limited to, AAV1, AAV2, AAV3, AAV4, AAV5, AAV6, AAV7, AAV8, AAV9, AAV10, AAV11, AAV12, artificial variants such as AAV.rhlO, AAV.rh32/33, AAV.rh43, AAV.rh64R1, rAAV2-retro, AAV-DJ, AAV-PHP.B, AAV-PHP.S, AAV-PHP.eB, or other engineered versions of AAV. In preferred forms, the AAV serotype used in the disclosed compositions and methods is AAV6 or AAV9. Other engineered AAVs that have been developed can be used for the purpose of introducing transgenes, and in the disclosed compositions and methods.

Introduction of gene editing compositions (e.g., RNA-guided endonuclease and the one or more AAV vectors) to the cell can be performed ex vivo and at the same or different times. The cell can be a T cell (e.g., CD8+ T cells such as effector T cells, memory T cells, central memory T cells, and effector memory T cells, or CD4+ T cells such as Th1 cells, Th2 cells, Th17 cells, and Treg cells), hematopoietic stem cell (HSC), macrophage, natural killer cell (NK), or dendritic cell (DC).

Also disclosed are isolated cells modified according to the foregoing methods. The cells can be modified to be bispecific or multispecific. A population of cells can be derived by expanding the isolated cells. Disclosed are pharmaceutical compositions containing the population of cells with a pharmaceutically acceptable buffer, carrier, diluent or excipient.

Also disclosed are methods of treatment. An exemplary method involves treating a subject having a disease, disorder, or condition by administering to the subject an effective amount of the aforementioned pharmaceutical composition. Disclosed is a method of treating a subject having a disease, disorder, or condition associated with an elevated expression or specific expression of an antigen by administering to the subject an effective amount of a T cell modified according to the disclosed methods to contain a CAR that targets the antigen.

Further disclosed is a method of treating a subject having a disease, disorder, or condition by administering to the subject an effective amount of a pharmaceutical composition having a genetically modified cell, where the cell is modified by introducing to the cell: (a) an RNA-guided endonuclease; and (b) one or more AAV vectors including (i) a sequence encoding one or more crRNAs that direct the RNA-guided endonuclease to one or more target genes; and (ii) one or more HDR templates containing a sequence that encodes one or more chimeric antigen receptors (CAR); and (iii) one or more sequences homologous to a target site.

In some forms, the pharmaceutical composition can include a population of cells derived by expanding the genetically modified cell. The genetically modified cell can be a T cell (e.g., CD8+ T cells such as effector T cells, memory T cells, central memory T cells, and effector memory T cells, or CD4+ T cells such as Th1 cells, Th2 cells, Th17 cells, and Treg cells), hematopoietic stem cell (HSC), macrophage, natural killer cell (NK), or dendritic cell (DC). The genetically modified cell can be bispecific or multispecific. The cell can have been isolated from the subject having the disease, disorder, or condition, or from a healthy donor, prior to genetic modification. Introduction of gene editing compositions (e.g., RNA-guided endonuclease and the one or more AAV vectors) to the cell can be performed ex vivo.

The CAR can target one or more antigens specific for or associated with the disease, disorder, or condition, which can be a cancer, an inflammatory disease, a neuronal disorder, HIV/AIDS, diabetes, a cardiovascular disease, an infectious disease, or an autoimmune disease. Exemplary cancers include, but are not limited to, chronic lymphocytic leukemia (CLL), acute lymphocytic leukemia (ALL), acute myeloid leukemia (AML), chronic myelogenous leukemia (CML), mantle cell lymphoma, non-Hodgkin's lymphoma, and Hodgkin's lymphoma. In preferred forms, other exemplary cancers include cancers listed in Table 4.

Exemplary antigens that can be targeted by the CAR include, but are not limited to, antigens listed in Table 3 such as AFP, AKAP-4, ALK, Androgen receptor, B7H3, BCMA, Bcr-Abl, BORIS, Carbonic, CD123, CD138, CD174, CD19, CD20, CD22, CD30, CD33, CD38, CD80, CD86, CEA, CEACAMS, CEACAM6, Cyclin, CYP1B1, EBV, EGFR, EGFR806, EGFRvIII, EpCAM, EphA2, ERG, ETV6-AML, FAP, Fos-related antigenl, Fucosyl, fusion, GD2, GD3, GloboH, GM3, gp100, GPC3, HER-2/neu, HER2, HMWMAA, HPV E6/E7, hTERT, Idiotype, IL12, IL13RA2, IM19, IX, LCK, Legumain, IgK, LMP2, MAD-CT-1, MAD-CT-2, MAGE, MelanA/MART1, Mesothelin, MET, ML-IAP, MUC1, Mutant p53, MYCN, NA17, NKG2D-L, NY-BR-1, NY-ESO-1, NY-ESO-1, OY-TES1, p53, Page4, PAP, PAX3, PAXS, PD-L1, PDGFR-β, PLAC1, Polysialic acid, Proteinase3 (PR1), PSA, PSCA, PSMA, Ras mutant, RGSS, RhoC, ROR1, SART3, sLe(a), Sperm protein 17, SSX2, STn, Survivin, Tie2, Tn, TRP-2, Tyrosinase, VEGFR2, WT1, and XAGE. The CAR can be an anti-CD19 CAR (e.g., CD19BBz) or an anti-CD22 CAR (e.g., CD22BBz). In some forms, the CAR can be bispecific or multivalent.

Preferably, the subject to be treated in accordance with any of the foregoing methods of treatment can be a human.

Additional advantages of the disclosed method and compositions will be set forth in part in the description which follows, and in part will be understood from the description, or can be learned by practice of the disclosed method and compositions. The advantages of the disclosed method and compositions will be realized and attained by means of the elements and combinations particularly pointed out in the appended claims. It is to be understood that both the foregoing general description and the following detailed description are exemplary and explanatory only and are not restrictive of the invention as claimed.

### BRIEF DESCRIPTION OF THE DRAWINGS

The accompanying drawings, which are incorporated in and constitute a part of this specification, illustrate several embodiments of the disclosed method and compositions and together with the description, serve to explain the principles of the disclosed method and compositions.

FIG. 1A is a schematic representation of the AAV-Cpf1 approach for generating chimeric antigen receptor (CAR) T cells. LbCpf1 mRNA electroporation is combined with AAV-delivery of multiple crRNAs and an HDR template encoding a CAR, thus enabling combinatorial knockout of different genes and targeted CAR knock-in in human primary T cells. FIG. 1B is a graph showing quantification of TRAC indel frequencies generated by AAV9-crTRAC-Cpf1 with a titration series of MOI. FIG. 1C is a graph showing quantification of TCR knockout frequencies generated by AAV6-crTRAC-Cpf1 with a titration series of MOI. Human primary CD4+ T cells were infected with AAV6 1e3 (n=2), AAV6 1e4 (n=2), or AAV6 1e5 (n=5). All comparisons are to the vector control. FIG. 1D is a schematic representation of an AAV6-crRNA array containing one U6 promoter, three LbCpf1 direct repeats (DRs) and two different crRNA cassettes targeting the PDCD1 and TRAC loci. FIG. 1E is a graph showing quantification of indel frequencies at TRAC and PDCD1 target sites 5 days after infection. In FIG. 1E, from left to right bars represent uninfected, unsorted and sorted respectively, for both TRAC and PDCD1. All comparisons are to the uninfected control. Unpaired T tests were used to assess significance. Data are shown as mean±s.e.m. with individual data points on the bar graph. * p<0.05; *** p<0.001.

FIG. 2A is a schematic representation of the AAV6 construct design for PDCD1^{KO};dTomato-TRAC^{KI} which mediates combinatorial PDCD1 knockout and dTomato transgene knock-in into the TRAC locus. FIG. 2B are representative flow cytometry plots showing dTomato knock-in frequencies at the TRAC locus 5 days post AAV transduction. FIG. 2C is a graph showing quantification of dTomato knock-in frequency at the TRAC target site (uninfected: n=2; AAV Vector: n=3; PDCD1^{KO};dTomato-TRAC^{KI}: n=3). FIG. 2D is a graph showing quantification of GFP knock-in frequency at the PDCD1 target site. FIG. 2E is a schematic representation of the AAV6 construct design for dTomato-TRAC^{KI};GFP-PDCD1^{KI} which mediates combinatorial dTomato and GFP knock-in into the TRAC and PDCD1 locus respectively. FIG. 2F is a graph showing quantification of percentages of GFP and dTomato single and double positive cells (AAV Vector: n=2; dTomato-TRAC^{KI};GFP-PDCD1^{KI}: n=4). From left to right, bars represent GFP⁺, dTomato⁺, and FITC⁺dTomato⁺, respectively, for both AAV vector and AAV-TRAC-PDCD1-DKI. FIG. 2G is a schematic representation of the two AAV6 vector system design for dual-targeting (PDCD1^{KO};dTomato-TRAC^{KI} and TRAC^{KI};GFP-PDCD1^{KI}). FIG. 2H is a graph showing quantification of percentages of GFP and dTomato single and double positive cells (biological replicates, n=3). From left to right, bars represent GFP⁺, dTomato⁺, and GFP⁺dTomato⁺, respectively, for both AAV vector and TRAC-KIKO PDCD1-KIKO. FIG. 2I is a graph showing quantification of TCR⁺ and TCR⁻ fractions in non-integration (Q4), single integration (Q1, Q3), and double integration (Q2) populations of T cells after transduction with the two-vector system. For both AAV vector and TRAC-KIKO PDCD1-KIKO, for each of Q1-Q4, TCR⁻ is represented by the shorter bar imposed over the taller TCR⁺ bar. All comparisons are to the vector control. Unpaired T tests were used to assess significance. ** p<0.01; *** p<0.001. Data are shown as mean±s.e.m. (with individual data points on the bar graph in all graphs except FIG. 2I).

FIG. 3A is a schematic representation of a single AAV construct PDCD1^{KO};CD22BBz-TRAC^{KI} for delivering a double-targeting crRNA array and an HDR template encoding a CD22BBz CAR. The HDR template contains an EFS-CD22BBz CAR-PA cassette, with the CD22BBz CAR transgene driven by an EFS promoter and terminated by a short polyA, flanked by two arms homologous to the TRAC locus. This construct mediates combinatorial CD22BBz CAR integration into the TRAC locus and PDCD1 knockout. FIG. 3B are representative flow cytometry plots of human primary CD4+ T cells showing CD22BBz CAR expression 5 days post AAV transduction. FIG. 3C is a graph showing quantification of CD22BBz CAR knock-in frequency in human primary CD4+ T cells. FIG. 3D is a graph showing quantification of HDR-mediated insertion of CD22BBz at the TRAC locus estimated by Nextera and Illumina sequencing. FIG. 3E is a graph showing quantification of NHEJ and HDR at the TRAC locus of T cells estimated by Nextera and Illumina sequencing. For the unsorted and sorted conditions, the three superimposed bars represent WT, NHEJ and HDR, from top to bottom respectively. FIG. 3F is a graph showing quantification of genomic knockout of PDCD1 in human primary CD4+ T cells mediated by the PDCD1^{KO};CD22BBz-TRAC^{KI} vector after one AAV6 transduction (day 5). All comparisons are to the vector control. Unpaired T tests were used to assess significance. *** p<0.001. Data are shown as mean±s.e.m. (with individual data points on the bar graph indicated as necessary). FIG. 3G is a graph showing a time-course analysis of CD22 CAR transgene retention after transduction. CAR22 expression levels of PDCD1^{KO};CD22BBz-TRAC^{KI} bulk targeted CAR-T cells were assayed by flow cytometry (biological replicates, n=3). The bulk T cells were stimulated once with mitomycin C-treated NALM6 cells (CD22⁺) 5 days post transduction. One-way ANOVA with Tukey's multiple comparisons test was used to assess significance. ** p<0.01; *** p<0.001. Data are shown as mean±s.e.m. with individual data points on the graph.

FIG. 4A are representative flow cytometry histograms showing the pattern of CD22BBz CAR transgene expression in T cells upon transduction with AAV-Cpf1 KIKO CAR-T or lentiviral CAR-T. CD22BBz KIKO generated bulk CAR-T cells with a more pronounced bimodal pattern of CAR transgene expression (clear CAR⁺ vs. CAR⁻ populations) compared to CD22BBz Lenti CAR which exhibited a continuous pattern (mixture of CAR⁺ vs. CAR⁻ populations). FIG. 4B is a graph showing time-course analysis of CAR transgene retention after transduction with either AAV-Cpf1 KIKO CAR-T or lentiviral CAR-T. CAR expression was measured by staining with a specific antibody followed by flow cytometry (n=3). At days 3 and 5, CD22BBz KIKO CAR is the lower bar while CD22BBz Lenti CAR is the higher bar, and at days 7 and 9 CD22BBz Lenti CAR is the lower bar while CD22BBz KIKO CAR is the higher bar. Two-way ANOVA with Sidak's multiple comparisons test was used to assess significance (multiple-testing corrected). KIKO vs. lentiviral CAR-T, ** p<0.01, *** p<0.001. FIG. 4C is a graph showing quantification of the cytotoxic activity of AAV-Cpf1 KIKO CAR-T and lentiviral CAR-T cells toward NALM6-GL cancer cells. Cell death was assayed through bioluminescence at different effector:target (E:T) ratios. At each E:T ratio, the three data points indicate CD22BBz KIKO CAR, CD22BBz Lenti CAR, and AAV Vector from top to bottom respectively. Data are shown as mean±s.e.m. KIKO vs. lentiviral CAR-T, ** p<0.01, *** p<0.001. FIG. 4D is a graph showing quantification of cell exhaustion markers (PD-1, TIGIT and LAGS) in AAV-Cpf1 KIKO CAR-T and lentiviral CAR-T cells. FIG. 4E is a graph showing quantification of effector cytokine production in AAV-Cpf1 KIKO CAR-T and lentiviral CAR-T cells. IFNγ and TNF-α production was tested by intracellular staining after co-culture with NALM6 for 5 hours (n=3). Data are shown as mean±s.e.m., with individual data points on the bar graph. In FIGS. 4D-4E, for each indicated marker, bars represent AAV-Vector, CD22BBz KIKO, and CD22BBz Lenti, from left to right respectively. For FIGS. 4C-E, two-way ANOVA with Tukey's multiple comparisons tests were used to assess significance (multiple-testing corrected).

FIG. 5A is a schematic representation of a single AAV construct designated TRAC^{KO};CD19BBz-PDCD1^{KI}, for delivering a double-targeting crRNA array and an HDR template encoding a CD19BBz CAR. This construct mediates combinatorial CD19BBz CAR integration into the PDCD1 locus and TRAC knockout. FIG. 5B is a graph showing quantification of CD19BBz CAR knock-in frequency in the PDCD1 target site (uninfected, n=2; AAV Vector, n=3; TRAC^{KO};CD19BBz-PDCD1^{KI}, n=3). Unpaired t test was used to assess significance. Vector vs. CD19BBz-KIKO, *** p<0.001. Data are shown as mean±s.e.m., with individual data points on the bar graph.

FIG. 5C are schematic representations of the two-vector system (PDCD1^{KO};CD22BBz-TRAC^{KI} and TRAC^{KO}; CD19BBz-PDCD1^{KI} for AAV-Cpf1 mediated CD19BBz CAR and CD22BBz CAR double knock-in. FIG. 5D is a graph showing quantification of percentages of CD19BBz− and CD22BBz− single and double positive cells (n=3). For each condition indicated, from left to right bars represent CD19 CAR+, CD22 CAR+, and CD19&CD22CAR+, respectively. Two-way ANOVA with Sidak's multiple comparisons test was used to assess significance (multiple-testing corrected). Vector vs. dual-targeting, *** p<0.001. Data are shown as mean±s.e.m., with individual data points on the bar graph. FIG. 5E is a graph showing quantification of TCR⁺ and TCR⁻ fractions in non-integration (Q4), single integration (Q1, Q3), and double integration (Q2) populations of T cells after transduction with the two-vector system (n=3). For both AAV vector and CD19BBz-KIKO CD22BBz-KIKO, for each of Q1-Q4, TCR⁻ is represented by the shorter bar imposed over the taller TCR⁺ bar. An unpaired t test was used to assess significance. TCR⁻ population, Vector vs. dual-targeting, * p<0.05, ** p<0.01, *** p<0.001. Data are shown as mean±s.e.m. FIG. 5F is a graph showing quantification of the cytotoxic activity of AAV-Cpf1 KIKO single and double knock-in CAR-T cells toward NALM6-GL cancer cells. Cell death was assayed through bioluminescence at the indicated effector:target (E:T) ratios. At the 1:1 ratio, the four data points represent CAR19 KIKO, CAR22;CAR19 KIKO, CAR22 KIKO, and Vector from top to bottom respectively. FIG. 5G is a graph showing quantification of effector cytokine production in AAV-Cpf1 KIKO single and double knock-in CAR-T cells. IFNγ and TNF-α production was assayed by intracellular staining after co-culture with NALM6 (n=3). For each indicated marker, bars represent Vector, CAR19⁺, CAR22⁺, and CAR19⁺;CAR22⁺, from left to right respectively. In FIGS. 5F-5G, data are shown as mean±s.e.m. (with individual data points shown on the bar graph as necessary). ** p<0.01, *** p<0.001.

FIGS. 6A-6B are column graphs showing quantification of percentages of CD19CAR− and CD22CAR− single and double positive cells generated by the AAV-Cpf1 (FIG. 6A) and AAV-Cas9 RNP (FIG. 6B) methods (biological replicates, n=2-3). For each indicated condition in FIGS. 6A and 6B, bars represent CAR19⁺, CAR22⁺, and CAR19⁺;CAR22⁺, from left to right respectively. Two-way ANOVA with Sidak's multiple comparisons test was used to assess significance (multiple-testing corrected). Vector vs. Cpf1 CAR19;CAR22 double knock-in: for CAR19+ cells, *** p<0.001; for CAR22+ cells, *** p<0.001; for CAR19+CAR22+ cells, *** p<0.001. Vector vs. Cas9 CAR19;CAR22 double knock-in: for CAR19+ cells, *** p<0.001; for CAR22+ cells, n.s., p=0.5471; for CAR19⁺;CAR22⁺ cells, * p<0.05. FIGS. 6C-6D are graphs showing time-course analyses of double CAR transgene retention after transduction by the AAV-Cpf1 (FIG. 6C) and AAV-Cas9 RNP (FIG. 6D) methods (biological replicates, n=3-4). Bulk T cells were stimulated once with target cells at 5 days post transduction. In FIGS. 6D-6F, data are shown as mean±s.e.m. with individual data points on the bar graph. One-way ANOVA with Tukey's multiple comparisons test was used to assess significance. ** p<0.01; *** p<0.001.

FIG. 7A is a graph showing quantification of the cytotoxic activity of CD22BBz KIKO CAR-T cells and Cas9 RNP CD22BBz CAR-T cells toward NALM6-GL cancer cells. Cell death was assayed through bioluminescence at the indicated effector:target (E:T) ratios. At the 1:1 ratio, the three data points represent Cpf1 KIKO CD22BBz, Cas9 RNP CD22BBz, and AAV Vector from top to bottom respectively. Cpf1 CD22BBz CAR vs. Vector, *** p<0.001; Cas9 RNP CD22BBz CAR vs. Vector, *** p<0.001; Cpf1 vs Cas9, n.s., not significant. FIG. 7B is a graph showing quantification of effector cytokine production by CD22BBz KIKO CAR-T cells and Cas9 RNP CD22BBz CAR-T cells. IFNγ and TNF-α production was assayed by intracellular staining after co-culture with NALM6 for 5 hours at E:T=1:1 (n=3). IFNγ group: Vector vs. Cas9, *** p<0.001; Vector vs. Cpf1, *** p<0.001; Cas9 vs. Cpf1, p=0.4835. TNF-α group: Vector vs. Cas9, *** p<0.001; Vector vs. Cpf1, *** p<0.001; Cas9 vs. Cpf1, p=0.1318. FIG. 7C is a graph showing quantification of cell exhaustion markers (PD-1, TIGIT and LAG3) in Cpf1 CD22BBz KIKO CAR-T cells and Cas9 RNP CD22BBz CAR-T cells. PD-1 group: Vector vs. Cas9, *** p<0.001; Vector vs. Cpf1, p=0.9087; Cas9 vs. Cpf1, *** p<0.001. TIGIT group: Vector vs. Cas9, *** p<0.001; Vector vs. Cpf1, *** p<0.001; Cas9 vs. Cpf1, *** p<0.001. LAG3 group: Vector vs. Cas9, *** p<0.001; Vector vs. Cpf1, *** p<0.001; Cas9 vs. Cpf1, *** p<0.001. For each indicated marker in FIGS. 7B and 7C, bars represent Vector, Cas9 RNP CD22BBz, and Cpf1 KIKO CD22BBz from left to right respectively. All data are shown as mean±s.e.m. with individual data points on the graph. In FIGS. 7A-7C, two-way ANOVA with Tukey's multiple comparisons test was used to assess significance (multiple-testing corrected).

FIG. 8 is a schematic representation of a workflow for CAR-T generation and functional testing using the AAV-Cpf1 KIKO system.

### DETAILED DESCRIPTION OF THE INVENTION

The disclosed method and compositions can be understood more readily by reference to the following detailed description of particular embodiments and the Examples included therein and to the Figures and their previous and following description.

As a “living drug,” genetically engineered CAR-T cells show promise for potent and specific anti-tumor activity in the clinic (Porter, D L., et al., N. Engl. J. Med., 365(8): 725-733 (2011); Kalos, M. et al., Sci. Transl. Med., 3(95):95ra73 (2011); Neelapu, S S., et al., N. Engl. J. Med. 377:2531-2544 (2017)). Currently, there are only two FDA-approved CAR-T platforms (Yescarta/axicabtagene ciloleucel, and Kymriah/tisagenlecleucel) for adult patients with certain types of large B-cell lymphoma such as non-Hodgkin lymphoma (NHL), and/or B-cell acute lymphoblastic leukemia (B-ALL) (Labanieh, L., et al., Nature Biomedical Engineering 2:377 (2018)). Most other leukemias and solid cancers do not have FDA-approved CAR-T therapy available, although multiple pre-clinical and clinical trials have been on-going for testing various forms of CAR-Ts (Rosenbaum, L., N. Engl. J. Med., 377:1313-1315 (2017)).

Multiple considerations are important for the generation of CAR T cells. One such aspect is the manufacturing process, which involves primary T cell isolation from a patient or a healthy donor, CAR transgene introduction, and expansion (Levine, B L., et al., Mol. Ther. Methods Clin. Dev., 4:92-101 (2017)). Therefore, transduction efficiency, transgene expression levels, and CAR stability or retention, are important aspects of this process. However, in traditional lentiviral or retroviral transduction, CAR-T cells tend to lose their transgenes and, therefore, the ability to recognize and destroy cancer cells (Ellis, J., Human Gene Therapy., 16:1241-1246 (2005)).

As shown in the Examples, the inventors have developed a novel approach to genome modification in general, and CAR T cell development in particular. This system, referred to herein as KIKO utilizes an AAV vector carrying both a Cpf1 crRNA array for flexible multiplexed editing and an HDR construct for introduction of a CAR, thereby holding a significant advantage over the Cas9 based system. Compared to Cas9 based T cell targeting, the AAV-Cpf1 system generates double knock-in CAR-Ts more efficiently. The PDCD1;TRAC dual-targeting CD22-specific KIKO CAR-T cells generated by the AAV-Cpf1 system have potency comparable to cells generated by the Cas9 based method in cytokine production and cancer cell killing, while expressing lower levels of exhaustion markers. Moreover, the AAV-Cpf1 KIKO method is simple, which potentiates large-scale manufacturing, and modular, which enables sophisticated genomic (e.g., T-cell) targeting. The KIKO system is readily scalable to high-dimensional CAR-T engineering such as dual-targeting with two CARs and bi-specifics (Fry, T J. et al., Nat. Med., 24:20-28 (2018); Majzner, R G. & Mackall, C L. Cancer Discov., 8(10):1219-1226 (2018)), as well as introduction of regulatory proteins such as proteins containing an auto-regulatory motif, kill-switch, effector booster, or dampener (Labanieh, L., et al., Nature Biomedical Engineering 2:377 (2018)).

While both viral- and non-viral methods for CAR-T engineering and genome editing are viable, the AAV-Cpf1 system combines both. Delivery of the RNA-guided endonuclease (RGN) is mediated by the transient expression of Cpf1 mRNA, and delivery of the crRNA and HDR template is mediated by stable AAV. This reduces the potentially unwanted continuous induction of double-stranded breaks by the RGN, while maintaining the need for stable presence of the HDR template and crRNA to achieve higher knock-in efficiency. As demonstrated in the Examples, the simple design of KIKO CAR does not sacrifice other features; rather, it improves CAR stability, transgene expression, effector function, and cancer cell killing ability, while reducing cell exhaustion. The comparative study described in the Examples showed that the AAV-Cpf1 KIKO method generates double knock-ins more efficiently than a current Cas9-based method with RNP electroporation and AAV-delivered HDR donors. The single knock-in, double knockout CAR T cells generated by the AAV-Cpf1 system express lower levels of exhaustion markers as compared to those generated by Cas9. These might be due, for example, to the higher efficiency of Cpf1 for generating multiple knock-in and knockout simultaneously when compared to a Cas9-based approach in T cells. These two RGNs are fundamentally different in terms of their mechanism of action and therefore do not have strict parity. After rigorous evaluation of toxicity profiles, it is contemplated that the AAV-Cpf1 KIKO method has the potential to improve “off-the-shelf” adoptive T cell therapies in the clinic.

Disclosed are methods of modifying the genome of a cell by introducing to the cell an RNA-guided endonuclease and one or more AAV vectors. At least one (preferably all) of the AAV vectors can include a sequence that encodes one or more crRNAs, where the one or more crRNAs collectively direct the RNA-guided endonuclease to one or more target genes.

Also disclosed are isolated cells modified according to the disclosed methods. In some forms, the cell is bispecific or multispecific. Also disclosed are populations of cells derived by expanding cells modified according to the disclosed methods. Also disclosed are pharmaceutical compositions comprising a population of cells derived by expanding cells modified according to the disclosed methods and a pharmaceutically acceptable buffer, carrier, diluent or excipient.

Also disclosed are methods of treating a subject having a disease, disorder, or condition comprising administering to the subject an effective amount of a pharmaceutical composition comprising a population of cells derived by expanding cells modified according to the disclosed methods and a pharmaceutically acceptable buffer, carrier, diluent or excipient.

Also disclosed are methods of treating a subject having a disease, disorder, or condition associated with an elevated expression or specific expression of an antigen, the method comprising administering to the subject an effective amount of a T cell modified according to the disclosed methods, where the T cell comprises a CAR that targets the antigen.

Also disclosed are method of treating a subject having a disease, disorder, or condition comprising administering to the subject an effective amount of a pharmaceutical composition comprising a genetically modified cell, where the cell is genetically modified by a method comprising introducing to the cell: (a) an RNA-guided endonuclease; and (b) one or more AAV vectors at least one of which comprises (i) a sequence that encodes one or more crRNAs, wherein the one or more crRNAs collectively direct the RNA-guided endonuclease to one or more target genes; and (ii) one or more HDR templates at least one of which comprises a sequence that encodes one or more chimeric antigen receptors (CAR); and (iii) one or more sequences at least one of which is homologous to a target site.

In some forms, two or more of the crRNAs can be encoded by a crRNA array. In some forms, each of the two or more crRNAs encoded by the crRNA array can direct the RNA-guided endonuclease to a different target gene. In some forms, two AAV vectors are introduced to the cell.

In some forms, at least one of the AAV vectors includes one or more HDR templates. In some forms, at least one of the HDR templates comprises: (a) a sequence that encodes a reporter gene, a chimeric antigen receptor (CAR), or combinations thereof; and (b) one or more sequences collectively homologous to one or more target sites. In some forms, the sequence in (a) further comprises a promoter and/or polyadenylation signal operationally linked to the reporter gene and the CAR.

In some forms, the RNA-guided endonuclease induces disruption of the target genes and/or the one or more HDR templates mediate targeted integration of the reporter gene, the CAR, or a combination thereof, at the target sites. In some forms, the target site is within the locus of the disrupted gene. In some forms, the target site is at a locus different from the disrupted gene. In some forms, the target gene or target site comprises PDCD1, TRAC, or genes/sites listed in Table 2.

In some forms of the method, the PDCD1 or TRAC gene is disrupted, the PDCD1 and TRAC genes are disrupted, the reporter gene, CAR, or combination thereof, is integrated in the PDCD1 or TRAC gene, the reporter genes, CARs, or combination thereof is integrated in both the PDCD1 and TRAC genes, the PDCD1 gene is disrupted and the reporter gene, CAR, or combination thereof, is integrated in the TRAC gene, or the TRAC gene is disrupted and the reporter gene, CAR, or combination thereof, is integrated in the PDCD1 gene.

In some forms, the CAR targets one or more antigens specific for cancer, an inflammatory disease, a neuronal disorder, HIV/AIDS, diabetes, a cardiovascular disease, an infectious disease, an autoimmune disease, or combinations thereof. In some forms, the CAR is bispecific or multivalent. In some forms, the CAR targets one or more antigens selected from Table 3. In some forms, the CAR is anti-CD19 or anti-CD22. In some forms, the CAR is CD19BBz or CD22BBz.

In some forms, the RNA-guided endonuclease is provided as an mRNA that encodes the RNA-guided endonuclease, a viral vector that encodes the RNA-guided endonuclease, or an RNA-guided endonuclease protein or a complex of the RNA-guided endonuclease protein and RNA. In some forms, the mRNA comprises pseudouridine, a 5′ cap, a poly(A) tail, a nuclear localization signal, or combinations thereof. In some forms, the mRNA is codon optimized for expression in a eukaryotic cell. In some forms, the mRNA is electroporated or transfected into the cell, or delivered to the cell via nanoparticles.

In some forms, the RNA-guided endonuclease is Cpf1 or an active variant, derivative, or fragment thereof. In some forms, the Cpf1 is derived from Lachnospiraceae bacterium ND2006 (LbCpf1), Francisella novicida U112 (FnCpf1), Acidaminococcus sp. BV3L6 (AsCpf1), Lachnospiraceae bacterium MA2020 (Lb2Cpf1), Lachnospiraceae bacterium MC2017 (Lb3Cpf1), Moraxella bovoculi 237 (MbCpf1), Butyrivibrio proteoclasticus (BpCpf1), Parcubacteria bacterium GWC2011_GWC2_44_17 (PbCpf1); Peregrinibacteria bacterium GW2011_GWA_33_10 (PeCpf1), Leptospira inadai (LiCpf1), Smithella sp. SC_K08D17 (SsCpf1), Porphyromonas crevioricanis (PcCpf1), Porphyromonas macacae (PmCpf1), Candidatus Methanoplasma termitum (CMtCpf1), Eubacterium eligens (EeCpf1), Moraxella bovoculi 237 (MbCpf1), Prevotella disiens (PdCpf1), or a bacterial species listed in Table 1. In some forms, the Cpf1 is a wildtype protein, a humanized Cpf1, a variant, a derivative, a fragment, a shuffled domain version, or combinations thereof. In some forms, the Cpf1 is LbCpf1, or an active variant, derivative, or fragment thereof.

In some forms, at least one of the AAV vectors is AAV6, AAV9, or any of the naturally occurring, artificial, or engineered AAV serotypes disclosed herein.

In some forms, the introduction is performed ex vivo. In some forms, the RNA-guided endonuclease and the one or more AAV vectors are introduced to the cell at the same or different times. In some forms, after introduction of one gene editing composition (e.g., RNA guided endonuclease), the cells can be introduced with another gene editing composition (e.g., an AAV) vector either immediately, or after a certain period of time such as, about 1 h, about 2 h, about 3 h, about 4 h, about 5 h, about 6 h, about 7 h, about 8 h, about 9 h, about 10 h, about 12 h, about 24 h, about 48 h, about 72 h, or about 96 h.

In some forms, the cell is a T cell, hematopoietic stem cell (HSC), macrophage, natural killer cell (NK), or dendritic cell (DC). In some forms, the T cell is a CD8+ T cell selected from the group consisting of effector T cells, memory T cells, central memory T cells, and effector memory T cells. In some forms, the T cell is a CD4+ T cell selected from the group consisting of Th1 cells, Th2 cells, Th17 cells, and Treg cells.

In some forms, the cell is isolated from the subject having the disease, disorder, or condition prior to the introduction to the cell. In some forms, the cell is isolated from a healthy donor prior to the introduction to the cell. In some forms, the introduction to the cell is performed ex vivo. In some forms, the pharmaceutical composition comprises a population of cells derived by expanding the genetically modified cell. In some forms, the subject is a human.

It is to be understood that the disclosed compositions and methods advantageously allow for simultaneous or combinatorial disruption (e.g., knockout (KO)) of one or more target genes and targeted integration (knock-in (KI)) of one or more HDR templates (e.g., a template encoding a reporter gene, CAR or combinations thereof). The targeted integration (non-random integration) allows for stable expression of the HDR template encoded gene (e.g., CAR) from a desired or intended locus, under the control of an endogenous or exogenous regulatory element (e.g., promoter). It will also be appreciated by the skilled person, that in the absence of the crRNAs, the RNA guided endonuclease shows no, minimal, or substantially reduced endonuclease activity towards the genome. Upon contact with the crRNAs, the endonuclease is directed to the targeted genes to induce cleavage of the DNA, and the HDR template undergoes homologous recombination at the target site induced by the DNA cleavage. Considered in this light, it will be appreciated that the disclosed methods and compositions advantageously facilitate multiplex gene editing (simultaneous KO and KI) at one or more loci in one-step.

### I. Definitions

“Introduce” in the context of genome modification refers to bringing in to contact. For example, to introduce a gene editing composition to a cell is to provide contact between the cell and the composition. The term encompasses penetration of the contacted composition to the interior of the cell by any suitable means, e.g., via transfection, electroporation, transduction, gene gun, nanoparticle delivery, etc.

“Homologous” refers to the sequence similarity or sequence identity between two polypeptides or between two nucleic acid molecules. When a position in both of the two compared sequences is occupied by the same base or amino acid monomer subunit, e.g., if a position in each of two DNA molecules is occupied by adenine, then the molecules are homologous at that position. The percent of homology between two sequences is a function of the number of matching or homologous positions shared by the two sequences divided by the number of positions compared×100. For example, if 6 of 10 of the positions in two sequences are matched or are homologous, then the two sequences are 60% homologous. By way of example, the DNA sequences ATTGCC and TATGGC share 50% homology. Generally, a comparison is made when two sequences are aligned to give maximum homology.

The term “operably linked” or “operationally linked” refers to functional linkage between a regulatory sequence (e.g., promoter, enhancer, silencer, polyadenylation signal, 5′ or 3′ untranslated region (UTR), splice acceptor, IRES, triple helix, 2A self-cleaving peptides such as F2A, E2A, P2A and T2A) and a heterologous nucleic acid sequence permitting them to function in their intended manner (e.g., resulting in expression of the latter). The term encompasses positioning of a regulatory region (sequence), a sequence to be transcribed, and/or a sequence to be translated in a nucleic acid so as to influence transcription or translation of such a sequence. The regulatory sequence can be positioned at any suitable distance from the sequence being regulated (e.g., 1 nucleotide-10,000 nucleotides). For example, to bring a coding sequence under the control of a promoter, the translation initiation site of the translational reading frame of the polypeptide is typically positioned between one and about fifty nucleotides downstream of the promoter. A promoter can, however, be positioned as much as about 5,000 nucleotides upstream of the translation initiation site or about 2,000 nucleotides upstream of the transcription start site. A promoter typically comprises at least a core (basal) promoter.

The term “antigen” as used herein is defined as a molecule capable of being bound by an antibody or T-cell receptor. An antigen can additionally be capable of provoking an immune response. This immune response can involve either antibody production, or the activation of specific immunologically-competent cells, or both. The skilled artisan will understand that any macromolecule, including virtually all proteins or peptides, can serve as an antigen. Furthermore, antigens can be derived from recombinant or genomic DNA. A skilled artisan will understand that any DNA, which comprises a nucleotide sequences or a partial nucleotide sequence encoding a protein that elicits an immune response therefore encodes an “antigen” as that term is used herein. Furthermore, one skilled in the art will understand that an antigen need not be encoded solely by a full length nucleotide sequence of a gene. It is readily apparent that the disclosed compositions and methods includes, but is not limited to, the use of partial nucleotide sequences of more than one gene and that these nucleotide sequences are arranged in various combinations to elicit the desired immune response. Moreover, a skilled artisan will understand that an antigen need not be encoded by a “gene” at all. It is readily apparent that an antigen can be generated synthesized or can be derived from a biological sample. Such a biological sample can include, but is not limited to a tissue sample, a tumor sample, a cell or a biological fluid. In the context of cancer, “antigen” refers to an antigenic substance that is produced in a tumor cell, which can therefore trigger an immune response in the host. These cancer antigens can be useful as markers for identifying a tumor cell, which could be a potential candidate/target during treatment or therapy. There are several types of cancer or tumor antigens. There are tumor specific antigens (TSA) which are present only on tumor cells and not on healthy cells, as well as tumor associated antigens (TAA) which are present in tumor cells and also on some normal cells. In some forms, the chimeric antigen receptors are specific for tumor specific antigens. In some forms, the chimeric antigen receptors are specific for tumor associated antigens. In some forms, the chimeric antigen receptors are specific both for one or more tumor specific antigens and one or more tumor associated antigens.

“Bi-specific chimeric antigen receptor” refers to a CAR that comprises two domains, wherein the first domain is specific for a first ligand/antigen/target, and wherein the second domain is specific for a second ligand/antigen/target. In some forms, the ligand is a B-cell specific protein, a tumor-specific ligand/antigen/target, a tumor associated ligand/antigen/target, or combinations thereof. A bispecific CAR is specific to two different antigens. A multi-specific or multivalent CAR is specific to more than one different antigen, e.g., 2, 3, 4, 5, or more. In some forms, a multi-specific or multivalent CAR targets and/or binds three or more different antigens.

“Encoding” or “encode” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

The terms “target nucleic acid,” “target sequence,” and “target site” refer to a nucleic acid sequence to which an oligonucleotide such as a gRNA is designed to specifically hybridize. The target nucleic acid has a sequence that is complementary to the nucleic acid sequence of the corresponding oligonucleotide directed to the target. The term target nucleic acid can refer to the specific subsequence of a larger nucleic acid to which the oligonucleotide is directed or to the overall sequence (e.g., a gene or mRNA). The difference in usage will be apparent from context.

As used herein, the term “locus” is the specific physical location of a DNA sequence (e.g. of a gene) on a chromosome. The term “locus” can refer to the specific physical location of an RNA guided endonuclease target sequence on a chromosome. Such a locus can comprise a target sequence that is recognized and/or cleaved by an RNA guided endonuclease. It is understood that a locus of interest can not only qualify a nucleic acid sequence that exists in the main body of genetic material (i.e. in a chromosome) of a cell but also a portion of genetic material that can exist independently to said main body of genetic material such as plasmids, episomes, virus, transposons or in organelles such as mitochondria as non-limiting examples.

“Isolated” means altered or removed from the natural state. For example, a nucleic acid or a peptide naturally present in a living animal is not “isolated,” but the same nucleic acid or peptide partially or completely separated from the coexisting materials of its natural state is “isolated.” An isolated nucleic acid or protein can exist in substantially purified form, or can exist in a non-native environment such as, for example, a host cell. An “isolated nucleic acid” refers to a nucleic acid segment or fragment which has been separated from sequences which flank it in a naturally occurring state, e.g., a DNA fragment which has been removed from the sequences which are normally adjacent to the fragment, i.e., the sequences adjacent to the fragment in a genome in which it naturally occurs. The term also applies to nucleic acids which have been substantially purified from other components which naturally accompany the nucleic acid, e.g., RNA or DNA or proteins, which naturally accompany it in the cell. The term therefore includes, for example, a recombinant DNA which is incorporated into a vector, into an autonomously replicating plasmid or virus, or into the genomic DNA of a prokaryote or eukaryote, or which exists as a separate molecule (i.e., as a cDNA or a genomic or cDNA fragment produced by PCR or restriction enzyme digestion) independent of other sequences. It also includes: a recombinant DNA which is part of a hybrid gene encoding additional polypeptide sequence, complementary DNA (cDNA), linear or circular oligomers or polymers of natural and/or modified monomers or linkages, including deoxyribonucleosides, ribonucleosides, substituted and alpha-anomeric forms thereof, peptide nucleic acids (PNA), locked nucleic acids (LNA), phosphorothioate, methylphosphonate, and the like.

In the context of cells, the term “isolated” also refers to a cell altered or removed from its natural state. That is, the cell is in an environment different from that in which the cell naturally occurs, e.g., separated from its natural milieu such as by concentrating to a concentration at which it is not found in nature. “Isolated cell” is meant to include cells that are within samples that are substantially enriched for the cell of interest and/or in which the cell of interest is partially or substantially purified.

As used herein, “transformed,” “transduced,” and “transfected” encompass the introduction of a nucleic acid or other material into a cell by one of a number of techniques known in the art.

A “vector” is a composition of matter which comprises an isolated nucleic acid and which can be used to deliver the isolated nucleic acid to the interior of a cell. Examples of vectors include but are not limited to, linear polynucleotides, polynucleotides associated with ionic or amphiphilic compounds, plasmids, and viruses. Thus, the term “vector” encompasses an autonomously replicating plasmid or a virus. The term is also construed to include non-plasmid and non-viral compounds which facilitate transfer of nucleic acid into cells, such as, for example, polylysine compounds, liposomes, and the like. Examples of viral vectors include, but are not limited to, adenoviral vectors, adeno-associated virus vectors, retroviral vectors, and the like.

“Tumor burden” or “tumor load” as used herein, refers to the number of cancer cells, the size or mass of a tumor, or the total amount of tumor/cancer in a particular region of a subject. Methods of determining tumor burden for different contexts are known in the art, and the appropriate method can be selected by the skilled person. For example, in some forms tumor burden can be assessed using guidelines provided in the Response Evaluation Criteria in Solid Tumors (RECIST).

As used herein, “subject” includes, but is not limited to, animals, plants, bacteria, viruses, parasites and any other organism or entity. The subject can be a vertebrate, more specifically a mammal (e.g., a human, horse, pig, rabbit, dog, sheep, goat, non-human primate, cow, cat, guinea pig or rodent), a fish, a bird or a reptile or an amphibian. The subject can be an invertebrate, more specifically an arthropod (e.g., insects and crustaceans). The term does not denote a particular age or sex. Thus, adult and newborn subjects, as well as fetuses, whether male or female, are intended to be covered. A patient refers to a subject afflicted with a disease or disorder. The term “patient” includes human and veterinary subjects.

The term “inhibit” or other forms of the word such as “inhibiting” or “inhibition” means to decrease, hinder or restrain a particular characteristic such as an activity, response, condition, disease, or other biological parameter. It is understood that this is typically in relation to some standard or expected value, i.e., it is relative, but that it is not always necessary for the standard or relative value to be referred to. “Inhibits” can also mean to hinder or restrain the synthesis, expression or function of a protein relative to a standard or control. Inhibition can include, but is not limited to, the complete ablation of the activity, response, condition, or disease. “Inhibits” can also include, for example, a 10% reduction in the activity, response, condition, disease, or other biological parameter as compared to the native or control level. Thus, the reduction can be about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100%, or any amount of reduction in between as compared to native or control levels. For example, “inhibits expression” means hindering, interfering with or restraining the expression and/or activity of the gene/gene product pathway relative to a standard or a control.

“Treatment” or “treating” means to administer a composition to a subject or a system with an undesired condition (e.g., cancer). The condition can include one or more symptoms of a disease, pathological state, or disorder. Treatment includes medical management of a subject with the intent to cure, ameliorate, stabilize, or prevent a disease, pathological condition, or disorder. This includes active treatment, that is, treatment directed specifically toward the improvement of a disease, pathological state, or disorder, and also includes causal treatment, that is, treatment directed toward removal of the cause of the associated disease, pathological state, or disorder. In addition, this term includes palliative treatment, that is, treatment designed for the relief of symptoms rather than the curing of the disease, pathological state, or disorder; preventative treatment, that is, treatment directed to minimizing or partially or completely inhibiting the development of the associated disease, pathological state, or disorder; and supportive treatment, that is, treatment employed to supplement another specific therapy directed toward the improvement of the associated disease, pathological state, or disorder. It is understood that treatment, while intended to cure, ameliorate, stabilize, or prevent a disease, pathological condition, or disorder, need not actually result in the cure, amelioration, stabilization or prevention. The effects of treatment can be measured or assessed as described herein and as known in the art as is suitable for the disease, pathological condition, or disorder involved. Such measurements and assessments can be made in qualitative and/or quantitative terms. Thus, for example, characteristics or features of a disease, pathological condition, or disorder and/or symptoms of a disease, pathological condition, or disorder can be reduced to any effect or to any amount.

“Prevention” or “preventing” means to administer a composition to a subject or a system at risk for an undesired condition (e.g., cancer). The condition can include one or more symptoms of a disease, pathological state, or disorder. The condition can also be a predisposition to the disease, pathological state, or disorder. The effect of the administration of the composition to the subject can be the cessation of a particular symptom of a condition, a reduction or prevention of the symptoms of a condition, a reduction in the severity of the condition, the complete ablation of the condition, a stabilization or delay of the development or progression of a particular event or characteristic, or reduction of the chances that a particular event or characteristic will occur.

As used herein, the terms “effective amount” or “therapeutically effective amount” means a quantity sufficient to alleviate or ameliorate one or more symptoms of a disorder, disease, or condition being treated, or to otherwise provide a desired pharmacologic and/or physiologic effect. Such amelioration only requires a reduction or alteration, not necessarily elimination. The precise quantity will vary according to a variety of factors such as subject-dependent variables (e.g., age, immune system health, weight, etc.), the disease or disorder being treated, as well as the route of administration, and the pharmacokinetics and pharmacodynamics of the agent being administered.

By “pharmaceutically acceptable” is meant a material that is not biologically or otherwise undesirable, i.e., the material can be administered to a subject along with the selected compound without causing any undesirable biological effects or interacting in a deleterious manner with any of the other components of the pharmaceutical composition in which it is contained.

As used herein, the terms “variant” or “active variant” refers to a polypeptide or polynucleotide that differs from a reference polypeptide or polynucleotide, but retains essential properties (e.g., functional or biological activity). A typical variant of a polypeptide differs in amino acid sequence from another, reference polypeptide. Generally, differences are limited so that the sequences of the reference polypeptide and the variant are closely similar overall and, in many regions, identical. A variant and reference polypeptide may differ in amino acid sequence by one or more modifications (e.g., substitutions, additions, and/or deletions). A substituted or inserted amino acid residue may or may not be one encoded by the genetic code. A variant of a polypeptide may be naturally occurring such as an allelic variant, or it may be a variant that is not known to occur naturally.

Modifications and changes can be made in the structure of the polypeptides of the disclosure and still obtain a molecule having similar characteristics as the polypeptide (e.g., a conservative amino acid substitution). For example, certain amino acids can be substituted for other amino acids in a sequence without appreciable loss of activity. Because it is the interactive capacity and nature of a polypeptide that defines that polypeptide's biological or functional activity, certain amino acid sequence substitutions can be made in a polypeptide sequence and nevertheless obtain a polypeptide with like properties (e.g., functional or biological activity).

Recitation of ranges of values herein are merely intended to serve as a shorthand method of referring individually to each separate value falling within the range, unless otherwise indicated herein, and each separate value is incorporated into the specification as if it were individually recited herein.

Use of the term “about” is intended to describe values either above or below the stated value in a range of approx. +/−10%; in other forms the values can range in value either above or below the stated value in a range of approx. +/−5%; in other forms the values can range in value either above or below the stated value in a range of approx. +/−2%; in other forms the values can range in value either above or below the stated value in a range of approx. +/−1%. The preceding ranges are intended to be made clear by context, and no further limitation is implied.

### II. Compositions

Compositions for use in the disclosed methods are provided. For example, gene editing compositions for use in methods of modifying the genome of a cell are disclosed. Pharmaceutical compositions containing the modified cells are also provided. As another example, pharmaceutical compositions for use in methods of treating a subject having a disease, disorder, or condition are disclosed. Also disclosed are compositions of modified cells (e.g., CAR T cells) for use in methods of treating a subject having a disease, disorder, or condition associated with an elevated expression or specific expression of an antigen. In some forms, the CAR targets the antigen exhibiting an elevated expression or specific expression in the disease, disorder, or condition.

A. Gene Editing Compositions

Gene editing compositions for use in methods of modifying the genome of a cell are disclosed. Exemplary gene editing compositions for modifying the genome of a cell include an RNA-guided endonuclease and a vector (e.g., AAV) containing a sequence (e.g., a crRNA array) that encodes one or more crRNAs that direct the endonuclease to one or more target genes. The RNA-guided endonuclease and vector (e.g., AAV) can be in the same or different compositions and can be introduced to the cell together or separately. For example, an RNA-guided endonuclease and vector (e.g., AAV) encoding one or more crRNAs can be provided in different compositions that are introduced to the cell together or separately. In some forms, after introduction of the RNA-guided endonuclease, the cells can be introduced with the AAV vector either immediately, or after a certain period of time such as, about 1 h, about 2 h, about 3 h, about 4 h, about 5 h, about 6 h, about 7 h, about 8 h, about 9 h, about 10 h, about 12 h, about 24 h, about 48 h, about 72 h, or about 96 h.

The RNA-guided endonuclease can alter (increase or reduce expression and/or activity) of one or more target genes (e.g., 1, 2, 3, 4, 5, 6, 7, 8, 9, 10, or more). For example, the RNA-guided endonuclease can cause disruption of one or more target genes (e.g., 1, 2, 3, 4, 5, 6, 7, 8, 9, 10, or more). This disruption includes but is not limited to alterations in the genome (such as, but not limited to, insertions, deletions, translocations, DNA or histone methylation, acetylation, and combinations thereof) resulting in reduced or abolished expression and/or activity of the target gene and/or gene product. Methods of determining the expression and/or activity of a gene product are known in the art. These include, but are not limited to, PCR, northern blot, southern blot, western blot, nuclease surveyor assays, sequencing, ELISA, FACS, mRNA-SEQ, single-cell RNA-SEQ, and other molecular biology, chemical, biochemical, cell biology, and immunology assays. A skilled person, based on methods known in the art, and the teachings provided herein would understand how to determine and/or confirm alteration of a target gene.

The RNA-guided endonuclease can be introduced to the cell through a variety of viral or non-viral techniques. For example, the RNA-guided endonuclease can be introduced via a viral vector (e.g., a retrovirus such as a lentivirus, adenovirus, poxvirus, Epstein-Barr virus, adeno-associated virus (AAV), etc.) that encodes the RNA-guided endonuclease. Non-viral approaches such as physical and/or chemical methods can also be used, including, but not limited to cationic liposomes and polymers, DNA nanoclew, gene gun, microinjection, electroporation, nucleofection, particle bombardment, ultrasound utilization, magnetofection, and conjugation to cell penetrating peptides. Such methods are described for example, in Nayerossadat N., et al., Adv. Biomed. Res., 1:27 (2012) and Lino C A, et al., Drug Deliv., 25(1):1234-1257 (2018). A skilled artisan, based on known delivery methods in the art (e.g., those disclosed in Nayerossadat N., et al., and Lino C A., et al) in context of their respective advantages and disadvantages, and the teachings disclosed herein, would be able to determine an optimal method for introduction of the RNA-guided endonuclease.

In preferred forms, the RNA-guided endonuclease can be provided to the cell via an mRNA that encodes the RNA-guided endonuclease. The mRNA can be modified or unmodified. The mRNA can be modified for example, to reduce immunogenicity, to optimize translation, and/or to confer increased stability and/or expression of the RNA-guided endonuclease. The modified mRNA can incorporate a number of chemical changes to the nucleotides, including changes to the nucleobase, the ribose sugar, and/or the phosphodiester linkage. These modified mRNA can improve efficiency of the RNA-guided endonuclease, reduce off-target effects, reduce toxicity, increase endonuclease protein levels, increase endonuclease activity, and/or increase mRNA stability relative to the unmodified mRNA. Li, B., et al., Nat. Biomed. Eng., 1(5): pii: 0066 (2017) and WO 2017/181107 disclose compositions and methods of modifying mRNAs that can be used in accordance with the compositions and methods disclosed herein.

The mRNA can contain modifications such as N6-methyladenosine (m6A), 5-methylcytosine (m5C), pseudouridine (ψ), N1-methylpseudouridine (me1ψ), and 5-methoxyuridine (5moU); a 5′ cap; a poly(A) tail; one or more nuclear localization signals; or combinations thereof.

The mRNA can be codon optimized for expression in a eukaryotic cell. The eukaryotic cell can be those of or derived from a particular organism, such as a plant or a mammal, including but not limited to human, or non-human eukaryote or animal or mammal, e.g., mouse, rat, rabbit, dog, livestock, or non-human mammal or primate. Codon-optimization describes gene engineering approaches that use changes of rare codons to synonymous codons that are more frequently used in the cell type of interest with the aim of increasing protein production. In general, codon optimization involves modifying a nucleic acid sequence for enhanced expression in the host cells of interest by replacing at least one codon (e.g., about or more than about 1, 2, 3, 4, 5, 10, 15, 20, 25, 50, or more codons) of the native sequence with codons that are more frequently or most frequently used in the genes of that host cell while maintaining the native amino acid sequence. Various species exhibit particular bias for certain codons of a particular amino acid. Codon bias (differences in codon usage between organisms) often correlates with the efficiency of translation of messenger RNA (mRNA), which is in turn believed to be dependent on, among other things, the properties of the codons being translated and the availability of particular transfer RNA (tRNA) molecules. The predominance of selected tRNAs in a cell is generally a reflection of the codons used most frequently in peptide synthesis. Accordingly, genes can be tailored for optimal gene expression in a given organism based on codon optimization. Codon usage tables are readily available, for example, at the “Codon Usage Database” available at www.kazusa.orjp/codon/ and these tables can be adapted in a number of ways. See Nakamura, Y., et al., Nucl. Acids Res., 28:292 (2000). Computer algorithms for codon optimizing a particular sequence for expression in a particular host cell are also available, such as Gene Forge (Aptagen; Jacobus, Pa.), are also available. In some forms, one or more codons (e.g., 1, 2, 3, 4, 5, 10, 15, 20, 25, 50, or more, or all codons) in a sequence encoding an RNA-guided endonuclease corresponds to the most frequently used codon for a particular amino acid.

The mRNA can be introduced to the cell via electroporation, nucleofection, transfection, and/or nanoparticle mediated delivery. In preferred forms, the mRNA is introduced to the cell via electroporation. Electroporation is temporary destabilization of the cell membrane by insertion of a pair of electrodes into it so that DNA molecules in the surrounding media of the destabilized membrane would be able to penetrate into cytoplasm and nucleoplasm of the cell. The RNA-guided endonuclease can also be introduced via direct electroporation of the endonuclease protein or endonuclease protein-RNA complex (e.g., endonuclease protein complexed with a crRNA).

1. RNA-Guided Endonuclease

An “RNA-guided endonuclease” is a polypeptide whose endonuclease activity and specificity depend on its association with an RNA molecule. The full sequence of this RNA molecule or more generally a fragment of this RNA molecule has the ability to specify a target sequence in the genome. In general, this RNA molecule has the ability to hybridize a target sequence and to mediate the endonuclease activity of the RNA-guided endonuclease. Non-limiting examples of RNA-guided endonucleases include Cas1, Cas1B, Cas2, Cas3, Cas4, Cas5, Cas6, Cas7, Cas8, Cas9 (also known as Csn1 and Csx12), Cpf1, homologues thereof, or modified versions thereof. A preferred RNA-guided endonuclease is Cas9 or Cas12a (Cpf1), both part of the CRISPR/Cas system.

CRISPR (Clustered Regularly Interspaced Short Palindromic Repeats) is an acronym for DNA loci that contain multiple, short, direct repetitions of base sequences. The prokaryotic CRISPR/Cas system has been adapted for use as gene editing (silencing, enhancing or changing specific genes) for use in eukaryotes (see, for example, Cong, Science, 15:339(6121):819-823 (2013) and Jinek, et al., Science, 337(6096):816-21 (2012)). By providing a cell with the required elements including a cas gene and specifically designed CRISPRs, the genome can be cut and modified at any desired location. Methods of preparing compositions for use in genome editing using CRISPR/Cas systems are described in detail in WO 2013/176772 and WO 2014/018423, which are specifically incorporated by reference herein in their entireties.

As used herein, the term “Cas” (CRISPR-associated) generally refers to an effector protein of a CRISPR-Cas system or complex. The term “Cas” can be used interchangeably with the terms “CRISPR” protein, “CRISPR-Cas protein,” “CRISPR effector,” CRISPR-Cas effector,” “CRISPR enzyme,” “CRISPR-Cas enzyme” and the like, unless otherwise apparent. The RNA-guided endonuclease can be a Cas effector Cas protein, or Cas enzyme. In general, a “CRISPR system,” “CRISPR-Cas system,” and “CRISPR complex” as used herein and in documents, such as WO 2014/093622 (PCT/US2013/074667), refers collectively to transcripts and other elements involved in the expression of or directing the activity of CRISPR-associated (“Cas”) genes, including sequences encoding a Cas gene, and where applicable, a tracr (trans-activating CRISPR) sequence (e.g. tracrRNA or an active partial tracrRNA), a tracr-mate sequence (encompassing a “direct repeat” and a tracrRNA-processed partial direct repeat in the context of an endogenous CRISPR system), a guide sequence (also referred to as a “spacer” in the context of an endogenous CRISPR system), or “RNA(s)” as that term is herein used (e.g., RNA(s) to guide Cas, such as Cas9 or Cpf1, e.g. CRISPR RNA (crRNA) and/or transactivating (tracr) RNA or a single guide RNA (sgRNA) (chimeric RNA)) or other sequences and transcripts from a CRISPR locus. In general, a CRISPR system is characterized by elements that promote the formation of a CRISPR complex at the site of a target sequence (also referred to as a protospacer in the context of an endogenous CRISPR system). See, e.g., Shmakov et al. (2015) “Discovery and Functional Characterization of Diverse Class 2 CRISPR-Cas Systems,” Molecular Cell, DOI: dx.doi.org/10.1016/j.molcel.2015.10.008.

The RNA-guided endonuclease can be a Cas effector protein selected from, without limitation, a type II, type V, or type VI Cas effector protein.

There are many resources available for helping practitioners determine suitable target sites once a desired DNA target sequence or target gene is identified. For example, numerous public resources, including a bioinformatically generated list of about 190,000 potential guide RNAs, targeting more than 40% of human exons, are available to aid practitioners in selecting target sites and designing the associated guide RNA to affect a nick or double strand break at the site. See also, crispr.u-psud.fr/, a tool designed to help scientists find CRISPR targeting sites in a wide range of species and generate the appropriate crRNA sequences.

In some forms, one or more elements of a CRISPR system are introduced into a target cell such that expression of the elements of the CRISPR system direct formation of a CRISPR complex at one or more target sites. While the specifics can be varied in different engineered CRISPR systems, the overall methodology is similar. A practitioner interested in using CRISPR technology to target a DNA sequence can insert a short DNA fragment containing the target sequence into a guide RNA expression plasmid. The sgRNA expression plasmid contains the target sequence (about 20 nucleotides), a form of the tracrRNA sequence (the scaffold) as well as a suitable promoter and necessary elements for proper processing in eukaryotic cells. Such vectors are commercially available (see, for example, Addgene). Many of the systems rely on custom, complementary oligomers that are annealed to form a double stranded DNA and then cloned into the sgRNA expression plasmid. Co-expression of the sgRNA and the appropriate Cas enzyme in the cell results in a single or double strand break (depending of the activity of the Cas enzyme) at the desired target site.

1.1 Cas12a (Cpf1)

Cas12s effector proteins include effector proteins derived from an organism from a genus comprising Streptococcus, Campylobacter, Nitratifractor, Staphylococcus, Parvibaculum, Roseburia, Neisseria, Gluconacetobacter, Azospirillum, Sphaerochaeta, Lactobacillus, Eubacterium, Corynebacter, Carnobacterium, Rhodobacter, Listeria, Paludibacter, Clostridium, Lachnospiraceae, Clostridiaridium, Leptotrichia, Francisella, Legionella, Alicyclobacillus, Methanomethyophilus, Porphyromonas, Prevotella, Bacteroidetes, Helcococcus, Letospira, Desulfovibrio, Desulfonatronum, Opitutaceae, Tuberibacillus, Bacillus, Brevibacilus, Methylobacterium or Acidaminococcus.

In some forms, the RNA-guided endonuclease (e.g., a Cpf1) comprises an effector protein (e.g., a Cpf1) from an organism from S. mutans, S. agalactiae, S. equisimilis, S. sanguinis, S. pneumonia; C. jejuni, C. coli; N. salsuginis, N. tergarcus; S. auricularis, S. carnosus; N. meningitides, N. gonorrhoeae; L. monocytogenes, L. ivanovii; C. botulinum, C. difficile, C. tetani, C. sordellii.

The RNA-guided endonuclease can comprise a chimeric effector protein comprising a first fragment from a first effector protein (e.g., a Cpf1) ortholog and a second fragment from a second effector (e.g., a Cpf1) protein ortholog, and wherein the first and second effector protein orthologs are different. At least one of the first and second effector protein (e.g., a Cpf1) orthologs can comprise an effector protein (e.g., a Cpf1) from an organism comprising Streptococcus, Campylobacter, Nitratifractor, Staphylococcus, Parvibaculum, Roseburia, Neisseria, Gluconacetobacter, Azospirillum, Sphaerochaeta, Lactobacillus, Eubacterium, Corynebacter, Carnobacterium, Rhodobacter, Listeria, Paludibacter, Clostridium, Lachnospiraceae, Clostridiaridium, Leptotrichia, Francisella, Legionella, Alicyclobacillus, Methanomethyophilus, Porphyromonas, Prevotella, Bacteroidetes, Helcococcus, Letospira, Desulfovibrio, Desulfonatronum, Opitutaceae, Tuberibacillus, Bacillus, Brevibacilus, Methylobacterium or Acidaminococcus; e.g., a chimeric effector protein comprising a first fragment and a second fragment wherein each of the first and second fragments is selected from a Cpf1 of an organism comprising Streptococcus, Campylobacter, Nitratifractor, Staphylococcus, Parvibaculum, Roseburia, Neisseria, Gluconacetobacter, Azospirillum, Sphaerochaeta, Lactobacillus, Eubacterium, Corynebacter, Carnobacterium, Rhodobacter, Listeria, Paludibacter, Clostridium, Lachnospiraceae, Clostridiaridium, Leptotrichia, Francisella, Legionella, Alicyclobacillus, Methanomethyophilus, Porphyromonas, Prevotella, Bacteroidetes, Helcococcus, Letospira, Desulfovibrio, Desulfonatronum, Opitutaceae, Tuberibacillus, Bacillus, Brevibacilus, Methylobacterium or Acidaminococcus wherein the first and second fragments are not from the same bacteria; for instance a chimeric effector protein comprising a first fragment and a second fragment wherein each of the first and second fragments is selected from a Cpf1 of S. mutans, S. agalactiae, S. equisimilis, S. sanguinis, S. pneumonia; C. jejuni, C. coli; N. salsuginis, N. tergarcus; S. auricularis, S. carnosus; N. meningitides, N. gonorrhoeae; L. monocytogenes, L. ivanovii; C. botulinum, C. difficile, C. tetani, C. sordellii; Francisella tularensis 1, Prevotella albensis, Lachnospiraceae bacterium MC20171, Butyrivibrio proteoclasticus, Peregrinibacteria bacterium GW2011_GWA2_33_10, Parcubacteria bacterium GW2011_GWC2_44_17, Smithella sp. SCADC, Acidaminococcus sp. BV3L6, Lachnospiraceae bacterium MA2020, Candidatus Methanoplasma termitum, Eubacterium eligens, Moraxella bovoculi 237, Leptospira inadai, Lachnospiraceae bacterium ND2006, Porphyromonas crevioricanis 3, Prevotella disiens and Porphyromonas macacae, wherein the first and second fragments are not from the same bacteria.

In some forms, the RNA-guided endonuclease is derived from a Cpf1 locus (herein, such RNA-guided endonucleases are also referred to as “Cpf1p”), e.g., a Cpf1 protein (and such RNA-guided endonuclease or Cpf1 protein or protein derived from a Cpf1 locus is also called “CRISPR enzyme”). In preferred forms, the Cpf1p is derived from a bacterial species selected from Francisella tularensis 1, Prevotella albensis, Lachnospiraceae bacterium MC2017_1, Butyrivibrio proteoclasticus, Peregrinibacteria bacterium GW2011_GWA2_33_10, Parcubacteria bacterium GW2011_GWC2_44_17, Smithella sp. SCADC, Acidaminococcus sp. BV3L6, Lachnospiraceae bacterium MA2020, Candidatus Methanoplasma termitum, Eubacterium eligens, Moraxella bovoculi 237, Leptospira inadai, Lachnospiraceae bacterium ND2006, Porphyromonas crevioricanis 3, Prevotella disiens and Porphyromonas macacae. In some forms, the Cpf1p is derived from a bacterial species selected from Acidaminococcus sp. BV3L6, Lachnospiraceae bacterium MA2020. In some forms, the effector protein is derived from a subspecies of Francisella tularensis 1, including but not limited to Francisella tularensis subsp. Novicida.

A preferred RNA-guided endonuclease is Cpf1, or a variant, derivative, or fragment thereof, such as, for example, Cpf1 derived from Francisella novicida U112 (FnCpf1), Acidaminococcus sp. BV3L6 (AsCpf1), Lachnospiraceae bacterium ND2006 (LbCpf1), Lachnospiraceae bacterium MA2020 (Lb2Cpf1), Lachnospiraceae bacterium MC2017 (Lb3Cpf1), Moraxella bovoculi 237 (MbCpf1), Butyrivibrio proteoclasticus (BpCpf1), Parcubacteria bacterium GWC2011_GWC2_44_17 (PbCpf1); Peregrinibacteria bacterium GW2011_GWA_33_10 (PeCpf1), Leptospira inadai (LiCpf1), Smithella sp. SC_K08D17 (SsCpf1), Porphyromonas crevioricanis (PcCpf1), Porphyromonas macacae (PmCpf1), Candidatus Methanoplasma termitum (CMtCpf1), Eubacterium eligens (EeCpf1), Moraxella bovoculi 237 (MbCpf1), or Prevotella disiens (PdCpf1). In some forms, the RNA guided endonuclease can be a Cpf1 ortholog, variant, or engineered derivative, derived from the bacterial species listed in Table 1. In some forms, the Cpf1 is a wildtype protein, a humanized Cpf1, a variant, a derivative, a fragment, a shuffled domain version, or combinations thereof. In some forms, the Cpf1 is LbCpf1, or a variant, derivative, or fragment thereof.

TABLE 1
List of bacterial species containing Cpf1 homologs.

| | |
|---|---|
| 1 | Acetonema longum DSM 6540 |
| 2 | Bacillus cereus IS075 |
| 3 | Moraxella bovoculi 237 |
| 4 | Prevotella bryantii B14 |
| 5 | Acinetobacter indicus |
| 6 | Succinivibrionaceae bacterium WG-1 |
| 7 | Prevotella disiens FB035-09AN |
| 8 | Helcococcus kunzii ATCC 51366 |
| 9 | Bacillus thuringiensis serovar finitimus YBT-020 |
| 10 | Francisella cf. novicida Fx1 |
| 11 | Listeria seeligeri FSL N1-067 |
| 12 | Flavobacterium branchiophilum FL-15 |
| 13 | Bacillus thuringiensis serovar sinensis |
| 14 | Leptospira inadai serovar Lyme str. 10 |
| 15 | Leptospira weilii str. Ecochallenge |
| 16 | Bacillus cereus AND1407 |
| 17 | Bacillus cereus BAG2O-3 |
| 18 | Bacillus cereus BAG3X2-1 |
| 19 | Bacillus cereus IS195 |
| 20 | Bacillus cereus IS845/00 |
| 21 | Francisella tularensis subsp. novicida FTE |
| 22 | Bacillus cereus Rock3-42 |
| 23 | Bacillus cereus AH1271 |
| 24 | Bacillus cereus AH1272 |
| 25 | Bacillus cereus AH1273 |
| 26 | Bacillus thuringiensis serovar monterrey BGSC 4AJ1 |
| 27 | Bacillus thuringiensis serovar tochigiensis BGSC 4Y1 |
| 28 | Bacillus thuringiensis serovar pulsiensis BGSC 4CC1 |
| 29 | Bacillus thuringiensis serovar pondicheriensis BGSC 4BA1 |
| 30 | Bacillus thuringiensis serovar andalousiensis BGSC 4AW1 |
| 31 | Bacillus cereus ISP3191 |
| 32 | Streptococcus sp. M143 |
| 33 | Francisella tularensis subsp. novicida FTG |
| 34 | Bacillus cereus 03BB102 |
| 35 | Francisella sp. TX076608 |
| 36 | Bacteroidetes oral taxon 274 str. F0058 |
| 37 | Collinsella tanakaei |
| 38 | Bacillus cereus biovar anthracis str. CI |
| 39 | Oribacterium sp. NK2B42 |
| 40 | uncultured bacterium (gcode 4) |
| 41 | Streptococcus sp. GMD2S |
| 42 | Streptococcus sp. GMD4S |
| 43 | Streptococcus sp. GMD6S |
| 44 | Anaerovibrio sp. RM50 |
| 45 | Capnocytophaga sp. oral taxon 336 str. F0502 |
| 46 | Bacillus anthracis str. BF1 |
| 47 | Bacillus cereus FRI-35 |
| 48 | Flavobacterium branchiophilum NBRC 15030 = ATCC 35035 |
| 49 | Moraxella lacunata NBRC 102154 |
| 50 | Streptococcus sp. GMD1S |
| 51 | Lachnospiraceae bacterium COE1 |
| 52 | Eubacterium sp. CAG:581 |
| 53 | Eubacterium sp. CAG:76 |
| 54 | Eubacterium eligens CAG:72 |
| 55 | Butyrivibrio sp. NC3005 |
| 56 | Pseudobutyrivibrio ruminis CF1b |
| 57 | Butyrivibrio fibrisolvens MD2001 |
| 58 | Porphyromonas crevioricanis JCM 15906 |
| 59 | Streptococcus oralis SK100 |
| 60 | Bacillus cereus MSX-A12 |
| 61 | Bacillus cereus MSX-D12 |
| 62 | Bacillus cereus VD102 |
| 63 | Bacillus cereus VDM053 |
| 64 | Bacillus cereus BAG3O-1 |
| 65 | Bacillus cereus B5-2 |
| 66 | Francisella hispaniensis FSC454 |
| 67 | Francisella noatunensis subsp. noatunensis FSC772 |
| 68 | Streptococcus oralis SK10 |
| 69 | Bacillus cereus 95/8201 |
| 70 | Acidaminococcus sp. BV3L6 |
| 71 | Bacteroides massiliensis B84634 = Timone 84634 = DSM 17679 = JCM 13223 |
| 72 | Moraxella caprae DSM 19149 |
| 73 | Porphyromonas macacae DSM 20710 = JCM 13914 |
| 74 | Prevotella albensis DSM 11370 = JCM 12258 |
| 75 | Proteocatella sphenisci DSM 23131 |
| 76 | Capnocytophaga sp. oral taxon 335 str. F0486 |
| 77 | Chryseobacterium taihuense |
| 78 | Flavobacterium branchiophilum |
| 79 | Porphyromonas macacae |
| 80 | Odoribacter splanchnicus |
| 81 | Moraxella ovis |
| 82 | Coprococcus eutactus |
| 83 | [Eubacterium] eligens |
| 84 | [Eubacterium] rectale |
| 85 | Eubacterium ventriosum |
| 86 | Ruminococcus bromii |
| 87 | Succiniclasticum ruminis |
| 88 | Francisella philomiragia |
| 89 | Moraxella equi |
| 90 | Bacillus pseudomycoides |
| 91 | uncultured bacterium |
| 92 | Moraxella caprae |
| 93 | Prevotella copri |
| 94 | [Bacillus thuringiensis] serovar konkukian |
| 95 | Bacillus thuringiensis serovar graciosensis |
| 96 | Bacillus thuringiensis serovar pingluonsis |
| 97 | Pseudobutyrivibrio xylanivorans |
| 98 | Bacillus anthracis |
| 99 | Francisella tularensis subsp. novicida |
| 100 | Moraxella bovis |
| 101 | Moraxella lacunata |
| 102 | Parabacteroides distasonis |
| 103 | Prevotella ruminicola |
| 104 | Acidaminococcus |
| 105 | Ruminococcus albus |
| 106 | Streptococcus oralis |
| 107 | Streptococcus pneumoniae |
| 108 | Butyrivibrio hungatei |
| 109 | Bacillus cereus |
| 110 | Bacillus lichenifomis |
| 111 | Bacillus thuringiensis |
| 112 | Alicyclobacillus acidoterrestris |
| 113 | Clostridioides difficile |
| 114 | Lactobacillus salivarius |
| 115 | Synergistes jonesii |
| 116 | Lachnospira pectinoschiza |
| 117 | Francisella philomiragia subsp. philomiragia ATCC 25017 |
| 118 | Francisella tularensis subsp. novicida U112 |
| 119 | Bacillus cereus AH187 |
| 120 | Bacillus cereus AH820 |
| 121 | Bacillus cereus W |
| 122 | Bacillus thuringiensis str. Al Hakam |
| 123 | Francisella tularensis subsp. novicida GA99-3549 |
| 124 | Bacillus cereus NVH0597-99 |
| 125 | Bacillus cereus H3081.97 |
| 126 | Bacillus cereus 03BB108 |
| 127 | Porphyromonas crevioricanis |
| 128 | Bacillus anthracis str. A0465 |
| 129 | [Eubacterium] eligens ATCC 27750 |
| 130 | Butyrivibrio proteoclasticus B316 |
| 131 | Capnocytophaga ochracea DSM 7271 |
| 132 | Bacillus cereus BGSC 6E1 |
| 133 | Bacillus cereus m1293 |
| 134 | Bacillus cereus BDRD-ST26 |
| 135 | Bacillus cereus ATCC 4342 |
| 136 | Chryseobacterium taichungense |
| 137 | Sneathia amnii |
| 138 | Bacillus cereus ATCC 10987 |
| 139 | Treponema porcinum |
| 140 | Bacillus cereus G9241 |
| 141 | [Bacillus thuringiensis] serovar konkukian str. 97-27 |
| 142 | Bacillus cereus E33L |
| 143 | Pseudomonas borbori |
| 144 | Eubacterium coprostanoligenes |
| 145 | Leptospira inadai serovar Lyme |
| 146 | Porphyromonas crevioricanis JCM 13913 |
| 147 | Bacteroides plebeius |
| 148 | Bacillus cereus NC7401 |
| 149 | Lactobacillus plantarum subsp. plantarum |
| 150 | Bacillus cereus F837/76 |
| 151 | Bacillus cereus Q1 |
| 152 | Prevotella stercorea |
| 153 | Bacteroides galacturonicus |
| 154 | Moraxella bovoculi |
| 155 | Candidatus Uhrbacteria bacterium CG11_big_fil_rev_8_21_14_0_20_41_9 |
| 156 | Candidatus Roizmanbacteria bacterium CG10_big_fil_rev_8_21_14_0_10_39_6 |
| 157 | Candidatus Roizmanbacteria bacterium CG11_big_fil_rev_8_21_14_0_20_37_16 |
| 158 | Candidatus Roizmanbacteria bacterium CG17_big_fil_post_rev_8_21_14_2_50_39_7 |
| 159 | Candidatus Roizmanbacteria bacterium CG22_combo_CG10-13_8_21_14_all_38_20 |
| 160 | Candidatus Ryanbacteria bacterium CG10_big_fil_rev_8_21_14_0_10_43_42 |
| 161 | Candidatus Taylorbacteria bacterium CG11_big_fil_rev_8_21_14_0_20_46_11 |
| 162 | Candidatus Terrybacteria bacterium CG10_big_fil_rev_8_21_14_0_10_41_10 |
| 163 | Candidatus Uhrbacteria bacterium CG_4_10_14_0_2_um_filter_41_7 |
| 164 | Candidatus Uhrbacteria bacterium CG_4_9_14_3_um_filter_41_35 |
| 165 | Candidatus Roizmanbacteria bacterium CG03_land_8_20_14_0_80_39_12 |
| 166 | candidate division WWE3 bacterium CG_4_9_14_0_2_um_filter_35_11 |
| 167 | candidate division WWE3 bacterium CG_4_9_14_3_um_filter_39_7 |
| 168 | candidate division WWE3 bacterium CG10_big_fil_rev_8_21_14_0_10_35_32 |
| 169 | candidate division WWE3 bacterium CG22_combo_CG10-13_8_21 14 all 39 12 |
| 170 | Candidatus Yonathbacteria bacterium CG_4_10_14_0_8_um_filter_47_645 |
| 171 | Candidatus Yonathbacteria bacterium CG_4_10_14_3_um_filter_47_65 |
| 172 | Candidatus Yonathbacteria bacterium CG_4_8_14_3_um_filter_46_25 |
| 173 | Candidatus Yonathbacteria bacterium CG_4_9_14_0_2_um_filter_47_74 |
| 174 | Candidatus Yonathbacteria bacterium CG_4_9_14_0_8_um_filter_46_47 |
| 175 | Candidatus Moranbacteria bacterium CG08_land_8_20_14_0_20_34_16 |
| 176 | Candidatus Gracilibacteria bacterium CG12_big_fil_rev_8_21_14_0_65_38_15 |
| 177 | Candidatus Gracilibacteria bacterium CG18_big_fil_WC_8_21_14_2_50_38_16 |
| 178 | Candidatus Kaiserbacteria bacterium CG_4_8_14_3_um_filter_38_9 |
| 179 | Candidatus Magasanikbacteria bacterium CG_4_10_14_0_8_um_filter_32_14 |
| 180 | Candidatus Moranbacteria bacterium CG_4_10_14_3_um_filter_41_65 |
| 181 | Candidatus Moranbacteria bacterium CG_4_8_14_3_um_filter_34_16 |
| 182 | Candidatus Moranbacteria bacterium CG_4_8_14_3_um_filter_41_13 |
| 183 | Candidatus Moranbacteria bacterium CG_4_9_14_0_8_um_filter_41_43 |
| 184 | Candidatus Moranbacteria bacterium CG_4_9_14_3_um_filter_33_15 |
| 185 | Candidatus Yonathbacteria bacterium CG17_big_fil_post_rev_8_21_14_2_50_46_19 |
| 186 | Candidatus Moranbacteria bacterium CG17_big_fil_post_rev_8_21_14_2_50_41_107 |
| 187 | Candidatus Moranbacteria bacterium CG23_combo_of_CG06-09_8_20_14_all_41_28 |
| 188 | Candidatus Nealsonbacteria bacterium CG08_land_8_20_14_0_20_38_20 |
| 189 | Candidatus Peregrinibacteria bacterium CG_4_10_14_0_2_um_filter_41_8 |
| 190 | Candidatus Peregrinibacteria bacterium CG_4_9_14_0_2_um_filter_41_14 |
| 191 | Candidatus Roizmanbacteria bacterium CG_4_10_14_3_um_filter_39_13 |
| 192 | Candidatus Roizmanbacteria bacterium CG_4_9_14_0_2_um_filter_38_17 |
| 193 | Candidatus Roizmanbacteria bacterium CG_4_9_14_0_2_um_filter_39_13 |
| 194 | Clostridium sp. AF34-10BH |
| 195 | Bacillus sp. AFS094611 |
| 196 | bacterium HR35 |
| 197 | Candidatus Gracilibacteria bacterium |
| 198 | Clostridia bacterium |
| 199 | Lachnospiraceae bacterium GAM79 |
| 200 | Catenovulum sp. CCB-QB4 |
| 202 | Roseburia sp. OM02-15 |
| 203 | Acidaminococcus sp. AM33-14BH |
| 204 | Bacillus nitratireducens |
| 205 | Clostridium sp. AM34-9AC |
| 206 | Clostridium sp. AM42-36 |
| 207 | Coprococcus sp. AF16-22 |
| 208 | Coprococcus sp. AF16-5 |
| 209 | Coprococcus sp. AF19-8AC |
| 210 | Ruminococcus sp. AF37-3AC |
| 211 | Ruminococcus sp. AM28-29LB |
| 212 | Ruminococcus sp. AM36-18 |
| 213 | bacterium (Candidatus Gribaldobacteria) CG08_land_8_20_14_0_20_39_15 |
| 214 | Candidatus Yonathbacteria bacterium CG23_combo_of_CG06-09_8_20_14_all_46_18 |
| 215 | Bacillus sp. MB353a |
| 216 | Flavobacteriales bacterium TMED235 |
| 217 | Gammaproteobacteria bacterium TMED134 |
| 218 | Bacteroidetes bacterium HGW-Bacteroidetes-12 |
| 219 | Bacteroidetes bacterium HGW-Bacteroidetes-6 |
| 220 | bacterium (Candidatus Gribaldobacteria) CG_4_10_14_0_2_um_filter_33_15 |
| 221 | bacterium (Candidatus Gribaldobacteria) CG_4_9_14_3_um_filter_33_9 |
| 222 | bacterium (Candidatus Gribaldobacteria) CG07_land_8_20_14_0_80_33_18 |
| 223 | Candidatus Gracilibacteria bacterium CG_4_9_14_0_2_um_filter_38_7 |
| 224 | Parcubacteria group bacterium CG_4_9_14_0_2_um_filter_41_8 |
| 225 | Parcubacteria group bacterium CG10_big_fil_rev_8_21_14_0_10_41_35 |
| 226 | Parcubacteria group bacterium CG11_big_fil_rev_8_21_14_0_20_41_14 |
| 227 | Moraxella sp. VT-16-12 |
| 228 | Leptospira sp. FH1-B-C1 |
| 229 | Leptospira sp. FH1-B-B1 |
| 230 | Prevotella sp. P4-119 |
| 231 | Prevotella sp. P4-98 |
| 232 | Parcubacteria group bacterium GW2011_GWC2_44_17 |
| 233 | Butyrivibrio sp. YAB3001 |
| 234 | Candidatus Gracilibacteria bacterium HOT-871 |
| 235 | Bacillus sp. UMTAT18 |
| 236 | Treponema endosymbiont of Eucomonympha sp |
| 237 | Flavobacterium sp. 316 |
| 238 | candidate division WS6 bacterium OLB21 |
| 239 | Candidatus Roizmanbacteria bacterium GW2011_GWA2_37_7 |
| 240 | Candidatus Falkowbacteria bacterium GW2011_GWA2_41_14 |
| 241 | Parcubacteria group bacterium GW2011_GWA2_44_12 |
| 242 | Robinsoniella sp. RHS |
| 243 | Parcubacteria group bacterium GW2011_GWF2_44_17 |
| 244 | Candidatus Peregrinibacteria bacterium GW2011_GWA2_33_10 |
| 245 | Candidatus Peregrinibacteria bacterium GW2011_GWC2_33_13 |
| 246 | candidate division WS6 bacterium GW2011_GWA2_37_6 |
| 247 | Pedobacter sp. Leaf176 |
| 248 | Brumimicrobium aurantiacum |
| 249 | Bacillus sp. 112mf |
| 250 | Alteromonas sp. W12 |
| 251 | Succinivibrio dextrinosolvens H5 |
| 252 | Alicyclobacillus acidoterrestris ATCC 49025 |
| 253 | Francisella tularensis subsp. novicida PA10-7858 |
| 254 | Prevotella amnii DNF00058 |
| 255 | Prevotella disiens DNF00882 |
| 256 | Lachnospiraceae bacterium MA2020 |
| 257 | Lachnospiraceae bacterium MC2017 |
| 258 | Prevotella brevis ATCC 19188 |
| 259 | Lachnospiraceae bacterium NC2008 |
| 260 | Lachnospiraceae bacterium ND2006 |
| 261 | Thiomicrospira sp. XS5 |
| 262 | Arcobacter butzleri L348 |
| 263 | Francisella tularensis subsp. novicida F6168 |
| 264 | Bacillus cereus D17 |
| 265 | Streptococcus oralis subsp. dentisani |
| 266 | Helicobacter sp. 13S00482-2 |
| 267 | Smithella sp. SC_K08D17 |
| 268 | Bacteroidales bacterium KA00251 |
| 269 | Smithella sp. SCADC |
| 270 | Beggiatoa sp. 4572_84 |
| 271 | Firmicutes bacterium CAG_194_44_15 |
| 272 | Bacteroidetes bacterium |
| 273 | Candidatus Gracilibacteria bacterium GN02-872 |
| 274 | Leptospira sp. YH101 |
| 275 | Prevotella ihumii |
| 276 | Candidatus Saccharibacteria bacterium QS_5_54_17 |
| 277 | Sedimentisphaera cyanobacteriorum |
| 278 | Fibrobacter sp. UWH8 |
| 279 | Barnesiella sp. An22 |
| 280 | Eubacterium sp. CAG76_36_125 |
| 281 | Bdellovibrionales bacterium CG10_big_fil_rev_8_21_14_0_10_45_34 |
| 282 | Flavobacteriales bacterium CG_4_10_14_0_2_um_filter_32_8 |
| 283 | Ignavibacteriales bacterium CG_4_9_14_3_um_filter_30_11 |
| 284 | Candidatus Falkowbacteria bacterium CG11_big_fil_rev_8_21_14_0_20_39_10 |
| 285 | Candidatus Gottesmanbacteria bacterium CG_4_10_14_0_8_um_filter_37_24 |
| 286 | Candidatus Gottesmanbacteria bacterium CG11_big_fil_rev_8_21_14_0_20_37_11 |
| 287 | Candidatus Gottesmanbacteria bacterium CG23_combo_of CG06- |
| | 09_8 20_14_all_37_19 |
| 288 | Candidatus Gracilibacteria bacterium CG_4_10_14_0_8_um_filter_38_28 |
| 289 | Candidatus Wildermuthbacteria bacterium RIFCSPHIGHO2_02_FULL_45_25 |
| 290 | Clostridium sp. C105KSO15 |
| 291 | Bacteroidetes bacterium GWF2_33_38 |
| 292 | Bacteroidetes bacterium RIFOXYA12_FULL_33_9 |
| 293 | Bacteroidetes bacterium RIFOXYA2_FULL_33_7 |
| 294 | Candidatus Campbellbacteria bacterium RIFCSPLOWO2_01_FULL_34_15 |
| 295 | Candidatus Falkowbacteria bacterium RBG_13_39_14 |
| 296 | Nitrospinae bacterium RIFCSPLOWO2_02_FULL_39_110 |
| 297 | Candidatus Sungbacteria bacterium RIFCSPLOWO2_01_FULL_54_21 |
| 298 | Candidatus Wildermuthbacteria bacterium RIFCSPHIGHO2_01_FULL_45_20 |
| 299 | Lachnospiraceae bacterium OF09-6 |
| 300 | Candidatus Gracilibacteria bacterium CG1_02_38_174 |
| 301 | Candidatus Magasanikbacteria bacterium CG1_02_32_51 |
| 302 | Phycisphaerae bacterium SM-Chi-D1 |
| 303 | Acidaminococcus massiliensis |
| 304 | Bacillus wiedmannii |
| 305 | Streptococcus oralis subsp. oralis |
| 306 | Eubacterium sp. 41_20 |
| 307 | Odoribacter sp. 43_10 |

Cpf1 effector proteins can be modified, e.g., an engineered or non-naturally-occurring effector protein or Cpf1. In some forms, the modification can comprise mutation of one or more amino acid residues of the effector protein. The one or more mutations can be in one or more catalytically active domains of the effector protein. The effector protein can have reduced or abolished nuclease activity compared with an effector protein lacking said one or more mutations. In some forms, the effector protein does not direct cleavage of one or other DNA or RNA strand at the target locus of interest. In some forms, the effector protein does not direct cleavage of either DNA or RNA strand at the target locus of interest. In preferred forms, the one or more mutations can comprise two mutations. In preferred forms, the one or more amino acid residues are modified in a Cpf1 effector protein, e.g., an engineered or non-naturally-occurring effector protein or Cpf1. In preferred forms, the Cpf1 effector protein is an LbCpf1 effector protein. In some forms, the one or more modified or mutated amino acid residues are D917A, E1006A or D1255A with reference to the amino acid position numbering of the FnCpf1 effector protein. In some forms, the one or more mutated amino acid residues are D908A, E993A, and D1263A with reference to the amino acid positions in AsCpf1 or LbD832A, E925A, D947A, and D1180A with reference to the amino acid positions in LbCpf1.

In some forms, one or more mutations of the two or more mutations can be in a catalytically active domain of the effector protein comprising a RuvC domain. In some forms, the RuvC domain can comprise a RuvCI, RuvCII or RuvCIII domain, or a catalytically active domain which is homologous to a RuvCI, RuvCII or RuvCIII domain etc. or to any relevant domain as described in any of the herein described methods. The effector protein can comprise one or more heterologous functional domains. The one or more heterologous functional domains can comprise one or more nuclear localization signal (NLS) domains. The one or more heterologous functional domains can comprise at least two or more NLS domains. The one or more NLS domain(s) can be positioned at or near or in proximity to a terminus of the effector protein (e.g., Cpf1) and if two or more NLSs, each of the two can be positioned at or near or in proximity to a terminus of the effector protein (e.g., Cpf1) The one or more heterologous functional domains can comprise one or more transcriptional activation domains. In preferred forms, the transcriptional activation domain can comprise VP64. The one or more heterologous functional domains can comprise one or more transcriptional repression domains. In preferred forms, the transcriptional repression domain comprises a KRAB domain or a SID domain (e.g. SID4X). The one or more heterologous functional domains can comprise one or more nuclease domains. In preferred forms, a nuclease domain comprises Fok1.

In some forms, the one or more heterologous functional domains can have one or more of the following activities: methylase activity, demethylase activity, transcription activation activity, transcription repression activity, transcription release factor activity, histone modification activity, nuclease activity, single-strand RNA cleavage activity, double-strand RNA cleavage activity, single-strand DNA cleavage activity, double-strand DNA cleavage activity and nucleic acid binding activity. At least one or more heterologous functional domains can be at or near the amino-terminus of the RNA-guided endonuclease protein and/or wherein at least one or more heterologous functional domains is at or near the carboxy-terminus of the effector protein. The one or more heterologous functional domains can be fused to the RNA-guided endonuclease. The one or more heterologous functional domains can be tethered to the RNA-guided endonuclease. The one or more heterologous functional domains can be linked to the RNA-guided endonuclease by a linker moiety.

In some forms, a protospacer adjacent motif (PAM) or PAM-like motif directs binding of the RNA-guided endonuclease complex to the target locus of interest. In some forms, the PAM is 5′ TTN, where N is A/C/G or T and the effector protein is FnCpf1p. In some forms, the PAM is 5′ TTTV, where V is A/C or G and the effector protein is AsCpf1, LbCpf1 or PaCpf1p. In some forms, the PAM is 5′ TTN, where N is A/C/G or T, the effector protein is FnCpf1p, and the PAM is located upstream of the 5′ end of the protospacer. In some forms, the PAM is 5′ CTA, where the effector protein is FnCpf1p, and the PAM is located upstream of the 5′ end of the protospacer or the target locus. In some forms, an expanded targeting range for RNA-guided genome editing nucleases can be used, where the T-rich PAMs of the Cpf1 family allow for targeting and editing of AT-rich genomes.

In some forms, the RNA-guided endonuclease is engineered and can comprise one or more mutations that reduce or eliminate an endonuclease activity. The amino acid positions in the FnCpf1p RuvC domain include but are not limited to D917A, E1006A, E1028A, D1227A, D1255A, N1257A, D917A, E1006A, E1028A, D1227A, D1255A and N1257A. A putative second nuclease domain is known that is most similar to PD-(D/E)XK nuclease superfamily and HincII endonuclease like. The point mutations to be generated in this putative nuclease domain to substantially reduce nuclease activity include but are not limited to N580A, N584A, T587A, W609A, D610A, K613A, E614A, D616A, K624A, D625A, K627A and Y629A. In some forms, the mutation in the FnCpf1p RuvC domain is D917A or E1006A, wherein the D917A or E1006A mutation completely inactivates the DNA cleavage activity of the FnCpf1 effector protein. In other forms, the mutation in the FnCpf1p RuvC domain is D1255A, wherein the mutated FnCpf1 effector protein has significantly reduced nucleolytic activity.

The amino acid positions in the AsCpf1p RuvC domain include but are not limited to 908, 993, and 1263. In some forms, the mutation in the AsCpf1p RuvC domain is D908A, E993A, and D1263A, wherein the D908A, E993A, and D1263A mutations completely inactivates the DNA cleavage activity of the AsCpf1 RNA-guided endonuclease. The amino acid positions in the LbCpf1p RuvC domain include but are not limited to 832, 947 or 1180. In preferred forms, the mutation in the LbCpf1p RuvC domain is LbD832A, E925A, D947A or D1180A, wherein the LbD832A E925A, D947A or D1180A mutations completely inactivates the DNA cleavage activity of the LbCpf1 RNA-guided endonuclease.

Mutations can also be made at neighboring residues, e.g., at amino acids near those indicated above that participate in the nuclease activity. In some forms, only the RuvC domain is inactivated, and in other forms, another putative nuclease domain is inactivated, wherein the effector protein complex functions as a nickase and cleaves only one DNA strand. In some forms, the other putative nuclease domain is a HincII-like endonuclease domain. In some forms, two FnCpf1, AsCpf1 or LbCpf1 variants (each a different nickase) are used to increase specificity, two nickase variants are used to cleave DNA at a target (where both nickases cleave a DNA strand, while minimizing or eliminating off-target modifications where only one DNA strand is cleaved and subsequently repaired). In some forms, the Cpf1 effector protein cleaves sequences associated with or at a target locus of interest as a homodimer comprising two Cpf1 RNA-guided endonucleases. In some forms, the homodimer can comprise two Cpf1 effector protein molecules comprising a different mutation in their respective RuvC domains.

In some forms, two or more nickases can be used, in particular a dual or double nickase approach. In some forms, a single type FnCpf1, AsCpf1 or LbCpf1 nickase can be delivered, for example a modified FnCpf1, AsCpf1 or LbCpf1 or a modified FnCpf1, AsCpf1 or LbCpf1 nickase as described herein. This results in the target DNA being bound by two RNA-guided endonuclease nickases. In addition, it is also envisaged that different orthologs can be used, e.g., an FnCpf1, AsCpf1 or LbCpf1 nickase on one strand (e.g., the coding strand) of the DNA and an ortholog on the non-coding or opposite DNA strand. The ortholog can be, but is not limited to, a Cas9 nickase such as a SaCas9 nickase or a SpCas9 nickase. It can be advantageous to use two different orthologs that require different PAMs and can also have different guide requirements, thus allowing a greater deal of control for the user. In some forms, DNA cleavage will involve at least four types of nickases, wherein each type is guided to a different sequence of target DNA, wherein each pair introduces a first nick into one DNA strand and the second introduces a nick into the second DNA strand. In such methods, at least two pairs of single stranded breaks are introduced into the target DNA wherein upon introduction of first and second pairs of single-strand breaks, target sequences between the first and second pairs of single-strand breaks are excised. In some forms, one or both of the orthologs is controllable, i.e. inducible.

The Cas12a enzymes can further include dCpf1 fused to an adenosine or cytidine deaminase such as those disclosed in U.S. Provisional Application Nos. 62/508,293, 62/561,663, and 62/568,133, 62/609,949, and 62/610,065.

Additional Cas12a enzymes that can be delivered used the compositions disclosed herein are discussed in International Patent Application Nos. WO 2016/205711, WO 2017/106657, and WO 2017/172682.

Given the potential toxicity of the RNA-guided endonuclease within the cells, due to possible non-specific interactions with various RNAs in the cell or off-site targeting, some approaches can be taken to induce the nuclease activity of the RNA-guided endonuclease, such as Cpf1, transiently (e.g., mRNA electroporation), ideally during the life-span of the guide RNA into the cells.

In some forms, the RNA-guided endonuclease (such as Cpf1) can be expressed under a stabilized or inactive form, which is made active upon activation by an enzyme produced by the cell or destabilization of its polypeptide structure inside the cell. Conditional protein stability can be obtained for instance by fusion of the endonuclease to a stabilizing/destabilizing protein based, as a non-limiting example, on the FKBP/rapamycin system, where protein conformational change induced by a small molecule. Chemical or light induced dimerization of a protein partner fused to the endonuclease protein can also be used to lock or unlock the endonuclease.

2. AAV Vector

Exemplary gene editing compositions for modifying the genome of a cell include an RNA-guided endonuclease and a vector (e.g., AAV vector) containing a sequence (e.g., a crRNA array) that encodes one or more crRNAs that collectively direct the endonuclease to one or more target genes, and optionally, one or more HDR templates. The crRNA array can encode two or more crRNAs that direct the endonuclease to different target genes. In some forms, one or more (e.g., 1, 2, 3, 4, 5, or more) AAV vectors are introduced to the cell. The vectors (e.g., AAV vector) can contain one or more HDR templates. The HDR templates can include a sequence that encodes a reporter gene, a chimeric antigen receptor (CAR), or combinations thereof, and one or more sequences homologous to one or more target sites. The HDR template can further include a promoter and/or polyadenylation signal operationally linked to each reporter gene, CAR, or combination thereof.

Suitable vectors for inclusion in the gene editing compositions or for providing elements of the gene editing compositions include, without limitation, plasmids and viral vectors derived from, for example, bacteriophages, baculoviruses, retroviruses (such as lentiviruses), adenoviruses, poxviruses, Epstein-Barr viruses, and adeno-associated viruses (AAV). The viral vector can be derived from a DNA virus (e.g., dsDNA or ssDNA virus) or an RNA virus (e.g., an ssRNA virus). Numerous vectors and expression systems are commercially available from commercial vendors including Addgene, Novagen (Madison, Wis.), Clontech (Palo Alto, Calif.), Stratagene (La Jolla, Calif.), and Invitrogen/Life Technologies (Carlsbad, Calif.).

A preferred vector for inclusion in the gene editing compositions or for providing elements of the gene editing compositions (e.g., crRNAs, HDR templates) is an adeno-associated viral (AAV) vector. AAV is a non-pathogenic, single-stranded DNA virus that has been actively employed over the years for delivering therapeutic genes in both in vitro and in vivo systems (Choi, et al., Curr. Gene Ther., 5:299-310, (2005)). AAV belongs to the parvovirus family and is dependent on co-infection with other viruses, mainly adenoviruses, in order to replicate. Initially distinguished serologically, molecular cloning of AAV genes has identified hundreds of unique AAV strains in numerous species. Each end of the single-stranded DNA genome contains an inverted terminal repeat (ITR), which is the only cis-acting element required for genome replication and packaging. The single-stranded AAV genome contains three genes, Rep (Replication), Cap (Capsid), and aap (Assembly). These three genes give rise to at least nine gene products through the use of three promoters, alternative translation start sites, and differential splicing. These coding sequences are flanked by the ITRs. The Rep gene encodes four proteins (Rep78, Rep68, Rep52, and Rep40), while Cap expression gives rise to the viral capsid proteins (VP; VP1/VP2/VP3), which form the outer capsid shell that protects the viral genome, as well as being actively involved in cell binding and internalization. It is estimated that the viral coat is comprised of 60 proteins arranged into an icosahedral structure with the capsid proteins in a molar ratio of 1:1:10 (VP1:VP2:VP3).

Recombinant AAV (rAAV), which lacks viral DNA, is essentially a protein-based nanoparticle engineered to traverse the cell membrane, where it can ultimately traffic and deliver its DNA cargo into the nucleus of a cell. In the absence of Rep proteins, ITR-flanked transgenes encoded within rAAV can form circular concatemers that persist as episomes in the nucleus of transduced cells. Because recombinant episomal DNA does not integrate into host genomes, it will eventually be diluted over time as the cell undergoes repeated rounds of replication. This will eventually result in the loss of the transgene and transgene expression, with the rate of transgene loss dependent on the turnover rate of the transduced cell. These characteristics make rAAV ideal for certain gene therapy applications.

AAV can be advantageous over other viral vectors due to low toxicity (this can be due to the purification method not requiring ultra centrifugation of cell particles that can activate the immune response) and low probability of causing insertional mutagenesis because AAV does not integrate into the host genome (primarily remaining episomal). The sequences placed between the ITRs will typically include a mammalian promoter, gene of interest, and a terminator. In many cases, strong, constitutively active promoters are desired for high-level expression of the gene of interest. Commonly used promoters of this type include the CMV (cytomegalovirus) promoter/enhancer, EF1a (elongation factor 1a), SV40 (simian virus 40), chicken β-actin and CAG (CMV, chicken β-actin, rabbit β-globin). All of these promoters provide constitutively active, high-level gene expression in most cell types. Some of these promoters are subject to silencing in certain cell types, therefore this consideration should to be evaluated for each application.

One of skill in the art would understand that in some cases it can be advantageous for a transgene (being targeted for integration) to be kept under the control of an endogenous promoter (e.g., a promoter at or near the site of integration). For example, the HDR template (e.g., provided by the AAV vector) can contain a splice acceptor/donor, 2A peptide, and/or internal ribosome entry site (IRES) operationally linked to a transgene (e.g., reporter gene, CAR) to allow expression of the transgene in frame with a gene at the site of integration and/or under the control of the promoter at the site of integration. In other cases, it can be advantageous for the transgene to be under the control of an exogenous promoter, such as a constitutive promoter or an inducible promoter. In such cases, the HDR template (e.g., provided by the AAV vector) can contain a promoter (e.g., EFS or tetracycline-inducible promoter) operationally linked to a transgene (e.g., reporter gene, CAR). In some forms, the HDR template does not contain a promoter operationally linked to the transgene (e.g., reporter gene, CAR).

The AAV vector used in the disclosed compositions and methods can be a naturally occurring serotype of AAV including, but not limited to, AAV1, AAV2, AAV3, AAV4, AAV5, AAV6, AAV7, AAV8, AAV9, AAV10, AAV11, AAV12, artificial variants such as AAV.rhlO, AAV.rh32/33, AAV.rh43, AAV.rh64R1, rAAV2-retro, AAV-DJ, AAV-PHP.B, AAV-PHP.S, AAV-PHP.eB, or other engineered versions of AAV. In preferred forms, the AAV used in the disclosed compositions and methods is AAV6 or AAV9.

Twelve natural serotypes of AAV have thus far been identified, with the best characterized and most commonly used being AAV2. These serotypes differ in their tropism, or the types of cells they infect, making AAV a very useful system for preferentially transducing specific cell types. For example, AAV serotypes 1, 2, 5 or a hybrid capsid AAV1, AAV2, AAV5 or any combination thereof can be used for targeting brain or neuronal cells; AAV4 can be selected for targeting cardiac cells. AAV8 is useful for delivery to the liver cells. Researchers have further refined the tropism of AAV through pseudotyping, or the mixing of a capsid and genome from different viral serotypes. These serotypes are denoted using a slash, so that AAV2/5 indicates a virus containing the genome of serotype 2 packaged in the capsid from serotype 5. Use of these pseudotyped viruses can improve transduction efficiency, as well as alter tropism. For example, AAV2/5 targets neurons that are not efficiently transduced by AAV2/2, and is distributed more widely in the brain, indicating improved transduction efficiency.

Other engineered AAVs have also been developed and can be used for the purpose of introducing transgenes, and in the disclosed compositions and methods. These are well known in the art and are contemplated for use in the disclosed methods and compositions.

One of skill in the art would be able to determine the optimal AAV serotype to be used for the respective application. The AAV can be AAV1, AAV2, AAV3, AAV4, AAV5, AAV6, AAV7, AAV8, AAV9, AAV10, AAV11, AAV12, artificial variants such as AAV.rhlO, AAV.rh32/33, AAV.rh43, AAV.rh64R1, rAAV2-retro, AAV-DJ, AAV-PHP.B, AAV-PHP.S, and AAV-PHP.eB, or combinations thereof. In preferred forms, the AAV vector for inclusion in the gene editing compositions or for providing elements of the gene editing compositions (e.g., crRNAs, HDR templates) is AAV6 or AAV9.

In some forms, the one or more crRNAs and one or more HDR templates are present on one nucleic acid molecule, e.g., one vector, e.g., one viral vector, e.g., one AAV vector. In some forms, the one or more crRNAs is present on a first nucleic acid molecule, e.g. a first vector, e.g., a first viral vector, e.g., a first AAV vector; and one or more HDR templates are present on a second nucleic acid molecule, e.g., a second vector, e.g., a second vector, e.g., a second AAV vector. The first and second nucleic acid molecules can be AAV vectors, e.g., AAV6 or AAV9.

In some forms, the RNA-guided endonuclease, one or more crRNAs, and one or more HDR templates are present on one nucleic acid molecule, e.g., an AAV vector such as AAV6 or AAV9. In some forms, one of the RNA-guided endonuclease, the crRNAs, and the HDR templates are present on a first nucleic acid molecule, e.g., a first AAV vector; and a second and third of the RNA-guided endonuclease, the crRNAs, and the HDR templates are encoded on a second nucleic acid molecule, e.g., a second AAV vector. The first and second nucleic acid molecules can be AAV6 or AAV9 vectors.

One of skill in the art would understand that the packaging limit of the vector to be used would determine the number and combinations of gene editing elements (e.g., RNA-guided endonuclease, crRNAs, HDR templates, or combinations thereof) that can be provided by said vector. For example, AAV has a packaging limit of approximately 4.5 to 4.8 Kb. As such, attempts to package larger constructs will lead to significantly reduced virus production. In preferred forms, the RNA-guided endonuclease is introduced to the cell by a different means from the vector encoding the crRNAs and/or HDR templates. Introduction of gene editing compositions (e.g., RNA-guided endonuclease and the one or more AAV vectors containing the crRNAs and/or HDR templates) to the cell can be performed ex vivo and at the same or different times.

3. crRNAs/Guide RNAs

Provided as part of the gene editing compositions are one or more crRNAs that direct the endonuclease to one or more target genes. When two or more crRNAs are used (e.g., to direct the RNA-guided endonuclease to two or more target genes/sites), they can be provided individually or together in the form of a crRNA array. CRISPR arrays (crRNA arrays) contain alternating conserved repeats and spacers that are transcribed into a precursor CRISPR RNA (pre-crRNA) and processed into individual CRISPR RNAs (crRNAs, also generally called gRNAs). The crRNA array can encode two or more (e.g., 2, 3, 4, 5, 6, 7, 8, 9, 10, or more) crRNAs that direct the endonuclease to different target genes or target sites (e.g., 2, 3, 4, 5, 6, 7, 8, 9, 10, or more).

Similarly to the mRNA encoding the RNA-guided endonuclease, the crRNAs or gRNAs can be introduced to the cell by any suitable means such as a variety of viral or non-viral techniques. For example, the crRNAs can be provided in a viral vector (e.g., a retrovirus such as a lentivirus, adenovirus, poxvirus, Epstein-Barr virus, adeno-associated virus (AAV), etc.). Non-viral approaches such as physical and/or chemical methods can also be used, including, but not limited to cationic liposomes and polymers, exosomes, DNA nanoclew, gene gun, microinjection, electroporation, nucleofection, particle bombardment, ultrasound utilization, magnetofection, and conjugation to cell penetrating peptides. Such methods are described for example, in Nayerossadat N., et al., Adv. Biomed. Res., 1:27 (2012) and Lino C A, et al., Drug Deliv., 25(1):1234-1257 (2018). A skilled artisan, based on known delivery methods in the art (e.g., those disclosed in Nayerossadat N., et al., and Lino C A., et al) in context of their respective advantages and disadvantages, and the teachings disclosed herein, would be able to determine an optimal method for introduction of the crRNAs.

In some forms, when the gene editing compositions are administered as an isolated nucleic acid or are contained within an expression vector, the RNA-guided endonuclease (such as Cpf1) can be encoded by the same nucleic acid or vector as the gRNA sequences. Alternatively, or in addition, the RNA-guided endonuclease (such as Cpf1) can be encoded in a physically separate nucleic acid from the gRNA sequences or in a separate vector.

The crRNAs/gRNAs can each individually be contained in a composition and introduced to a cell individually or collectively. Alternatively, these components can be provided in a single composition for introduction to a cell. Preferably, the one or more crRNAs are provided in a single viral vector, e.g., an AAV6 or AAV9 vector.

In contrast to Cas9, Cpf1 is tracrRNA independent and requires only an approximately 42 nucleotide long crRNA, which has 20-23 nucleotides at its 3′ end complementary to the protospacer of the target DNA sequence. Cpf1-associated CRISPR arrays are processed into mature crRNAs without the requirement of an additional tracrRNA and when complexed with Cpf1, the Cpf1p-crRNA complex is sufficient to efficiently cleave target DNA by itself. The crRNAs described herein comprise a spacer sequence (or guide sequence) and a direct repeat sequence. The seed sequence, e.g. the seed sequence of an FnCpf1 guide RNA is approximately within the first 5 nt on the 5′ end of the spacer sequence (or guide sequence) and mutations within the seed sequence adversely affect cleavage activity of the Cpf1 effector protein complex.

In some forms, the crRNA sequence has one or more stem loops or hairpins and is 30 or more nucleotides in length, 40 or more nucleotides in length, or 50 or more nucleotides in length. In certain forms, the crRNA sequence is between 42 and 44 nucleotides in length. In some forms, the crRNA contains about 19 nucleotides of a direct repeat and between 23 and 25 nucleotides of spacer sequence.

The term “guide RNA,” refers to the polynucleotide sequence containing a putative or identified crRNA sequence or guide sequence. The guide RNA can be any polynucleotide sequence having sufficient complementarity with a target nucleic acid sequence to hybridize with the target nucleic acid sequence and direct sequence-specific binding of an RNA-guided endonuclease to the target nucleic acid sequence. In some forms, the degree of complementarity between a guide sequence and its corresponding target sequence, when optimally aligned using a suitable alignment algorithm, is about or more than about 50%, 60%, 75%, 80%, 85%, 90%, 95%, 97.5%, 99%, or more. Optimal alignment can be determined with the use of any suitable algorithm for aligning sequences, non-limiting example of which include the Smith-Waterman algorithm, the Needleman-Wunsch algorithm, algorithms based on the Burrows-Wheeler Transform (e.g. the Burrows Wheeler Aligner), ClustalW, Clustal X, BLAT, Novoalign (Novocraft Technologies, ELAND (Illumina, San Diego, Calif.), SOAP (available at soap.genomics.org.cn), and Maq (available at maq.sourceforge.net).

The guide RNA sequence can be configured as a single sequence or as a combination of one or more different sequences, e.g., a multiplex configuration (referred to as an array). Multiplex configurations can include combinations of two, three, four, five, six, seven, eight, nine, ten, or more different guide RNAs. For example, in the context of a viral vector, multiple crRNAs/gRNAs can be tandemly arranged, optionally separated by a nucleotide sequence such as a direct repeat. The multiplexed format can involve multiple gRNAs under the control of a single promoter (e.g., U6) designed in an array format such that multiple gRNA sequences can be simultaneously expressed. In some forms, each individual crRNA or gRNA guide sequence can target a different target.

Guide RNA (gRNA) sequences for use in the disclosed compositions and methods can be sense or anti-sense sequences. The specific sequence of the gRNA can vary, but, regardless of the sequence, useful guide RNA sequences will be those that minimize off-target effects, achieve high efficiency alteration of the targeted gene or target site. The length of the guide RNA sequence can vary from about 20 to about 60 or more nucleotides, for example about 20, about 21, about 22, about 23, about 24, about 25, about 26, about 27, about 28, about 29, about 30, about 31, about 32, about 33, about 34, about 35, about 36, about 37, about 38, about 39, about 40, about 45, about 50, about 55, about 60 or more nucleotides. In some forms, a guide sequence is about or more than about 5, 10, 11, 12, 13, 14, 15, 16, 17, 18, 19, 20, 21, 22, 23, 24, 25, 26, 27, 28, 29, 30, 35, 40, 45, 50, 75, or more nucleotides in length. In some forms, a guide sequence is less than about 75, 50, 45, 40, 35, 30, 25, 20, 15, 12, or fewer nucleotides in length. The ability of a guide sequence to direct sequence-specific binding of a nucleic acid-targeting complex to a target sequence can be assessed by any suitable assay.

In the context of formation of a CRISPR complex, “target sequence” refers to a sequence to which a guide sequence is designed to target, e.g. have complementarity, where hybridization between a target sequence and a guide sequence promotes the formation of a CRISPR complex. The section of the guide sequence through which complementarity to the target sequence is important for cleavage activity is referred to herein as the seed sequence. A target sequence can comprise any polynucleotide, such as DNA or RNA polynucleotides and is comprised within a target locus of interest.

Without wishing to be bound by theory, it is believed that the target sequence should be associated with a PAM (protospacer adjacent motif); that is, a short sequence recognized by the CRISPR complex. The precise sequence and length requirements for the PAM differ depending on the CRISPR enzyme used, but PAMs are typically 2-5 base pair sequences adjacent to the protospacer (that is, the target sequence). The skilled person will be able to identify further PAM sequences for use with a given RNA-guided endonuclease. Further, engineering of the PAM Interacting (PI) domain of an RNA-guided endonuclease can allow programing of PAM specificity to improve target site recognition fidelity, and increase the versatility of the Cas, e.g. Cpf1, genome engineering platform. Cas proteins, such as Cas9 proteins, can be engineered to alter their PAM specificity, for example as described in Kleinstiver, B P., et al., Nature., 523(7561):481-5 (2015).

In some forms, a protospacer adjacent motif (PAM) or PAM-like motif directs binding of the effector protein complex to the target locus of interest. In some forms, the PAM is 5′ TTN, where N is A/C/G or T and the RNA-guided endonuclease is FnCpf1p. In some forms, the PAM is 5′ TTTV, where V is A/C or G and RNA-guided endonuclease is AsCpf1, LbCpf1 or PaCpf1p. In some forms, the PAM is located upstream of the 5′ end of the protospacer. The Cpf1 RNA-guided endonuclease provides for an expanded targeting range for RNA-guided genome editing nucleases wherein the T-rich PAMs of the Cpf1 family allow for targeting and editing of AT-rich genomes.

3.1 Target Genes and Target Sites

The guide RNA can be a sequence complementary to a coding or a non-coding sequence (e.g., a target sequence, target site, or target gene). The gRNA sequences can be complementary to either the sense or anti-sense strands of the target sequences. They can include additional 5′ and/or 3′ sequences that may or may not be complementary to a target sequence. They can have less than 100% complementarity to a target sequence, for example 50%, 60%, 70%, 75%, 80%, 85%, 90%, or 95% complementarity.

Upon formation of a ribonucleoprotein complex with the crRNA, the RNA-guided endonuclease localizes to a sequence (e.g., a target sequence, target site, or target gene) and causes disruption of a target gene and/or one or more HDR templates can mediate targeted integration of a reporter gene, a CAR, or combinations thereof at a target site. A target site can be within the locus of the disrupted gene or at a locus different from the disrupted gene. For example, a target site can overlap with a portion of a gene such as, an enhancer, promoter, intron, exon, or untranslated region (UTR).

The disclosed gene editing compositions are generally applicable to the targeting and/or alteration (e.g., disruption) of any sequence of interest in the genome, including non-coding and coding regions. One of skill in the art would understand that the targeted sequences would depend on the application for which genome modification is being performed and appropriate crRNAs/gRNAs would be designed accordingly. For example, in the context of CAR T cells, it is desirable to generate standardized therapy in which allogeneic therapeutic cells are administered to a subject in need thereof. By allogeneic is meant that the cells used for treating patients are not originating from said patient but from a donor belonging to the same species, and as such are genetically dissimilar. However, host versus graft rejection (HvG) and graft versus host disease (GvHD) severely limit their use. In these contexts, it is desirable to generate CAR T cells in which proteins involved in HvG and GvHD have been disrupted. Accordingly, TCR alpha, TCR beta, one or more HLA genes, one or more major histocompatibility complex (MHC) genes, or combinations thereof can be targeted by the crRNAs/gRNAs.

Immune checkpoints proteins are a group of molecules expressed by T cells that effectively serve as “brakes” to down-modulate or inhibit an immune response Immune checkpoint molecules include, but are not limited to Programmed Death 1 (PD-1, also known as PDCD1 or CD279, accession number: NM_005018), Cytotoxic T-Lymphocyte Antigen 4 (CTLA-4, also known as CD152, GenBank accession number AF414120.1), LAG3 (also known as CD223, accession number: NM_002286.5), Tim3 (also known as HAVCR2, GenBank accession number: JX049979.1), BTLA (also known as CD272, accession number: NM_181780.3), BY55 (also known as CD160, GenBank accession number: CR541888.1), TIGIT (also known as IVSTM3, accession number: NM_173799), LAIR1 (also known as CD305, GenBank accession number: CR542051.1, SIGLEC10 (GenBank accession number: AY358337.1), 2B4 (also known as CD244, accession number: NM_001 166664.1), PPP2CA, PPP2CB, PTPN6, PTPN22, CD96, CRTAM, SIGLEC7, SIGLEC9, TNFRSF10B, TNFRSF10A, CASP8, CASP10, CASP3, CASP6, CASP7, FADD, FAS, TGFBRII, TGFRBRI, SMAD2, SMAD3, SMAD4, SMAD10, SKI, SKIL, TGIF1, M ORA, IL10RB, HMOX2, IL6R, IL6ST, EIF2AK4, CSK, PAG1, SIT1, FOXP3, PRDM1, BATF, GUCY1A2, GUCY1A3, GUCY1 B2, GUCY1 B3 which directly inhibit immune cells. For example, CTLA-4 is a cell-surface protein expressed on certain CD4 and CD8 T cells; when engaged by its ligands (B7-1 and B7-2) on antigen presenting cells, T-cell activation and effector function are inhibited. Thus the disclosed gene editing compositions can be used to target and inactivate any immune check-point protein, including but not limited to, the aforementioned immune check-point proteins, such as PD1 and/or CTLA-4.

Any gene in the cell's genome can be a target gene or contain a target site. In some forms, a gene listed in Table 2 below could be a target gene or target site.

TABLE 2
Non-limiting examples of target genes or target sites

| | | | | |
|---|---|---|---|---|
| AAA1 | ABCB1 | ABHD6 | ABO | ACE2 |
| ADORA2A | ADRB2 | AKAP5 | ALK | ANAPC4 |
| ARHGEF2 | ART3 | ATG16L1 | B3GALNT1 | BCHE |
| C15orf53 | CACNA2D2 | CARD16 | CASP3 | CBLB |
| CD200 | CD244 | CD247 | CD27 | CD3D |
| CD83 | CD86 | CD8B | CDC14B | CDH11 |
| CIITA | CISD1 | CLEC16A | CLK3 | CLSTN3 |
| CX3CL1 | CXCL13 | CXCR4 | CYP24A1 | DDAH1 |
| DHX37 | ECM1 | EEA1 | EMP1 | EPHA1 |
| F5 | FADS1 | FADS2 | FADS3 | FAM103A1 |
| FUT2 | GALC | GART | GCKR | GOLGA8A |
| HAVCR2 | HEBP1 | HFE | HFE2 | HHEX |
| IBD5 | ICOS | IFI6 | IFIH1 | IGF2BP2 |
| IL17RB | IL18R1 | IL18RAP | IL1B | IL1RN |
| IL4R | IL5 | IL6ST | IL7 | INPP5B |
| IRGM | ITGAM | ITGAV | ITGAX | ITPKA |
| KIR2DL1 | KIR2DL3 | KIR2DL4 | KIR2DS4 | KIR3DL1 |
| KLRC1 | KLRC3 | KLRC4 | LACC1 | LAG3 |
| LPP | LTBR | LY75 | LYN | MAGEH1 |
| NAA25 | NAT10 | NELL1 | NF1 | NID1 |
| NRP1 | NSF | OAS1 | OCLN | ODC1 |
| PDE4B | PDE5A | PDIA3 | PENK | PFKFB4 |
| PLCL2 | PLD3 | PLSCR1 | PLTP | PMAIP1 |
| PRKAR1A | PRODH2 | PSORS1C3 | PTGER4 | PTPN13 |
| RGS16 | RNPEP | RPL23A | RPL4 | RPL5 |
| SH2D2A | SLAMF1 | SLC10A4 | SLC11A1 | SLC12A2 |
| SLC30A7 | SLC30A8 | SLC34A2 | SLC35C1 | SLC39A6 |
| SPRED2 | SRGN | ST3GAL4 | STARD6 | STAT2 |
| TET3 | THADA | TIGIT | TIMMDC1 | TLR5 |
| TNFRSF13B | TNFRSF14 | TNFRSF18 | TNFRSF9 | TNFSF18 |
| TRAF1 | TRBC1 | TRBC2 | TRIB1 | TSC1 |
| UGT3A1 | UHRF1BP1 | UPK1A | VAPB | VAV2 |
| YDJC | YIPF1 | ZAP70 | ZBED2 | ZBTB32 |
| ACOXL | ACP2 | ACSL6 | ADA | ADGRG1 |
| ANKRD1 | APC | APOBEC3G | APPL1 | ARHGAP31 |
| BEX3 | BLK | BSN | BST2 | BTLA |
| CCDC80 | CCRL2 | CD101 | CD180 | CD2 |
| CD4 | CD5 | CD58 | CD70 | CD74 |
| CDKAL1 | CDKN2A | CDKN2B | CHRNA4 | CHRNA7 |
| CR1 | CSF2 | CSNK1D | CTLA4 | CTSC |
| DDX50 | DENND1B | DGKA | DGKQ | DGUOK |
| EPRS | ERAP1 | ERBB3 | EVI5 | EXTL2 |
| FAM104A | FAM69A | FCER1A | FCGR2B | FCRL3 |
| GOLGA8B | GOT2 | GPR65 | GPX4 | GSTP1 |
| HIP1 | HLA-C | HLA-DQA1 | HLA-DRB1 | HS6ST1 |
| IKZF1 | IL12A | IL12B | IL12RB1 | IL13 |
| IL2 | IL21 | IL23R | IL2RA | IL4 |
| INS | IPMK | IRF1 | IRF5 | IRF8 |
| ITPR3 | JAZF1 | KCNA4 | KCNJ11 | KIAA1109 |
| KIR3DL2 | KIR3DL3 | KLC1 | KLF6 | KLRB1 |
| LAIR2 | LAT | LAT2 | LEKR1 | LNPEP |
| MAN2A1 | MGAT5 | MMEL1 | MYO9B | MZB1 |
| NKX2-3 | NLRP1 | NOD2 | NOTCH2 | NPTN |
| ORMDL3 | P2RX4 | P4HA1 | PADI4 | PDCD1 |
| PHF19 | PHGDH | PKD1L3 | PLA2G7 | PLAT |
| PMF1- | PMPCA | POPDC3 | PPARG | PRDX5 |
| BGLAP | | | | |
| PTPN2 | PTPN22 | PTPRS | PTTG1 | RGS1 |
| RSBN1 | RUNX3 | SAE1 | SCAMP3 | SH2B3 |
| SLC15A2 | SLC20A1 | SLC22A5 | SLC26A2 | SLC29A4 |
| SLC44A2 | SLC4A7 | SLC9A8 | SPATS2L | SPHK2 |
| STAT4 | STRADB | TAGAP | TCF7L2 | TET2 |
| TLR6 | TMEM123 | TMEM154 | TNF | TNFAIP3 |
| TNFSF4 | TNIP1 | TOR3A | TP53 | TRAC |
| TSC2 | TSPAN13 | TSPAN3 | TXK | TYK2 |
| VDR | VPREB1 | WFDC12 | WFS1 | XBP1 |

In some forms, a targeted gene or target site is selected from CTLA4, PPP2CA, PPP2CB, PTPN6, PTPN22, PDCD1, LAG 3, HAVCR2, BTLA, CD160, TIGIT, CD96, CRTAM, LAIR1, SIGLEC7, SIGLEC9, CD244, TNFRSF10B, TNFRSF10A, CASP8, CASP10, CASP3, CASP6, CASP7, FADD, FAS, TGFBRII, TGFRBRI, SMAD2, SMAD3, SMAD4, SMAD10, SKI, SKIL, TGIF1, IL10RA, IL10RB, HMOX2, IL6R, IL6ST, EIF2AK4, CSK, PAG1, SIT1, FOXP3, PRDM1, BATF, GUCY1A2, GUCY1A3, GUCY1 B2, and GUCY1 B3.

In preferred forms, exemplary target genes or target sites include, but are not limited to, PDCD1, TRAC, and genes selected from Table 2. In some forms, the PDCD1 and/or TRAC gene can be disrupted; one or more reporter genes, one or more CARs, or combinations thereof can be integrated in the PDCD1 and/or TRAC gene; the PDCD1 gene can be disrupted and the one or more reporter genes, one or more CARs, or combinations thereof can be integrated in the TRAC gene; or the TRAC gene can disrupted and the one or more reporter genes, one or more CARs, or combinations thereof can be integrated in the PDCD1 gene.

4. HDR Templates

Provided as part of the gene editing compositions are one or more HDR templates (e.g., 1, 2, 3, 4, 5, 6, 7, 8, 9, 10, or more). The HDR template is a donor sequence that allows for incorporation of a specific alteration at a desired site. The alteration can be for example, a single nucleotide change, a multiple nucleotide change, a frameshift, insertion of an endogenous or exogenous gene of interest, and/or insertion of an epitope tag, mutation or other genomic modification. The one or more HDR templates can contain a sequence that encodes a reporter gene, a chimeric antigen receptor (CAR), another gene of interest, or combinations thereof, and one or more sequences homologous to one or more target sites. The HDR template can further include one or more regulatory elements, e.g., a promoter, enhancer, silencer, 5′ or 3′ untranslated region (UTR), splice acceptor, IRES, 2A self-cleaving peptides (e.g., F2A, E2A, P2A and T2A), triple helix, polyadenylation signal, or combinations thereof, operationally linked to each reporter gene, CAR, or combination thereof.

In some forms, when the gene editing compositions (e.g., HDR templates) are administered as an isolated nucleic acid or are contained within an expression vector, the RNA-guided endonuclease (such as Cpf1) can be encoded by the same nucleic acid or vector as the HDR templates. Alternatively, or in addition, the RNA-guided endonuclease (such as Cpf1) can be encoded in a physically separate nucleic acid from the HDR templates or in a separate vector. The HDR templates can each individually be contained in a composition and introduced to a cell individually or collectively. Alternatively, these components can be provided in a single composition for introduction to a cell. Preferably, the one or more HDR templates are provided in a single viral vector, e.g., an AAV vector packaged in AAV serotypes such as AAV6 or AAV9 vector.

The gene editing compositions can be used to introduce targeted double-strand breaks (DSB) in an endogenous DNA sequence. The DSB activates cellular DNA repair pathways, which can be harnessed to achieve desired DNA sequence modifications near the break site. This is of interest where the inactivation of endogenous genes can confer or contribute to a desired trait. In particular forms, homologous recombination with an HDR template sequence is promoted at the site of the DSB, in order to introduce a gene of interest, such as a reporter gene or CAR.

An HDR template can be contained in a separate vector or provided as a separate polynucleotide. In some forms, an HDR template is designed to serve as a template in homologous recombination, such as within or near a target sequence nicked or cleaved by a RNA-guided endonuclease as a part of a nucleic acid-targeting complex. An HDR template can be of any suitable length, such as about or more than about 10, 15, 20, 25, 50, 75, 100, 150, 200, 500, 1000, or more nucleotides in length. In some forms, the HDR template is complementary or homologous to a portion of a target sequence. When optimally aligned, an HDR template might overlap with one or more nucleotides of a target sequences (e.g., about or more than about 1, 5, 10, 15, 20, 25, 30, 35, 40, 45, 50, 60, 70, 80, 90, 100 or more nucleotides). In some forms, when a template sequence and a polynucleotide comprising a target sequence are optimally aligned, the nearest nucleotide of the template polynucleotide is within about 1, 5, 10, 15, 20, 25, 50, 75, 100, 200, 300, 400, 500, 1000, 5000, 10000, or more nucleotides from the target sequence.

In some forms, the HDR template contains the following components: a 5′ homology arm, a replacement sequence, and a 3′ homology arm. The homology arms provide for recombination into the chromosome, thus replacing a portion of the endogenous genomic sequence with the replacement sequence (e.g., reporter gene, CAR, or other gene of interest). In some forms, the homology arms flank the most distal cleavage sites. In some forms, the 3′ end of the 5′ homology arm is the position next to the 5′ end of the replacement sequence. In some forms, the 5′ homology arm can extend at least 10, 20, 30, 40, 50, 100, 200, 300, 400, 500, 600, 700, 800, 900, 1000, 1500, or 2000 nucleotides 5′ from the 5′ end of the replacement sequence. In some forms, the 5′ end of the 3′ homology arm is the position next to the 3′ end of the replacement sequence. In some forms, the 3′ homology arm can extend at least 10, 20, 30, 40, 50, 100, 200, 300, 400, 500, 600, 700, 800, 900, 1000, 1500, or 2000 nucleotides 3′ from the 3′ end of the replacement sequence.

In some forms, the HDR template is single stranded or double stranded. In some forms, the HDR template is DNA, e.g., double stranded DNA or single stranded DNA. In some forms, the HDR template alters the structure of the target position by participating in homologous recombination. In some forms, the HDR template alters the sequence of the target position. In some forms, the HDR template results in the incorporation of a modified, or non-naturally occurring nucleotide sequence into the target nucleic acid. An HDR template having homology with a target position in a target gene can be used to alter the structure of a target sequence. The HDR template can include sequence which results in: a change in sequence of 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100, or more nucleotides of the target sequence.

4.1 Reporter Genes

Preferably, the HDR template mediates integration of a gene of interest, such as a reporter gene at the target sequence. A reporter gene includes any gene that could be used as an indicator of a successful event, e.g., transfection, transduction, and/or recombination. Reporter genes can allow simple identification and/or measurement of such events. Reporter genes can be fused to regulatory sequences or genes of interest to report expression location or levels, or serve as controls, for example, standardizing transfection efficiencies. Reporter genes include genes that code for fluorescent protein and enzymes that convert invisible substrates to luminescent or colored products.

Examples of reporter genes include, but are not limited to, glutathione-S-transferase (GST), horseradish peroxidase (HRP), chloramphenicol acetyltransferase (CAT) beta-galactosidase, beta-glucuronidase, luciferase, green fluorescent protein (GFP), dTomato, HcRed, DsRed, cyan fluorescent protein (CFP), yellow fluorescent protein (YFP), and autofluorescent proteins including blue fluorescent protein (BFP).

Reporter genes also include selectable markers that confer the ability to grow in the presence of toxic compounds such as antibiotics or herbicides, which would otherwise kill or compromise the cell. A selectable marker can also confer a novel ability to utilize a compound, for example, an unusual carbohydrate or amino acid. Non-limiting examples of selectable markers include genes that confer resistance to Blasticidin, G418/Geneticin, Hygromycin B, Puromycin, or Zeocin.

4.2 Chimeric Antigen Receptors (CAR)

Preferably, the HDR template mediates integration of a gene of interest, such as a CAR at the target sequence Immunotherapy using T cells genetically engineered to express a chimeric antigen receptor (CAR) is rapidly emerging as a promising new treatment for haematological and non-haematological malignancies. CARs are engineered receptors that possess both antigen-binding and T-cell-activating functions. Based on the location of the CAR in the membrane of the cell, the CAR can be divided into three main distinct domains, including an extracellular antigen-binding domain, followed by a space region, a transmembrane domain, and the intracellular signaling domain. The antigen-binding domain, most commonly derived from variable regions of immunoglobulins, typically contains VH and VL chains that are joined up by a linker to form the so-called “scFv.” The segment interposing between the antigen-binding domain (e.g., scFv) and the transmembrane domain is a “spacer domain.” The spacer domain can include the constant IgG1 hinge-CH2-CH3 Fc domain. In some cases, the spacer domain and the transmembrane domain are derived from CD8. The intracellular signaling domains mediating T cell activation can include a CD3ζ co-receptor signaling domain derived from C-region of the TCR α and β chains and one or more costimulatory domains.

In some forms, the antigen-binding domain can be derived from an antibody. The term antibody herein refers to natural or synthetic polypeptides that bind a target antigen. The term includes polyclonal and monoclonal antibodies, including intact antibodies and functional (e.g., antigen-binding) antibody fragments, including Fab fragments, F(ab′)₂ fragments, Fab′ fragments, Fv fragments, recombinant IgG (rlgG) fragments, single chain antibody fragments, including single chain variable fragments (scFv), and single domain antibodies (e.g., sdAb, sdFv, nanobody) fragments. The term encompasses genetically engineered and/or otherwise modified forms of immunoglobulins, such as intrabodies, peptibodies, chimeric antibodies, fully human antibodies, humanized antibodies, and heteroconjugate antibodies, multispecific, e.g., bispecific, antibodies, diabodies, triabodies, and tetrabodies, tandem di-scFv, tandem tri-scFv. The term also encompasses intact or full-length antibodies, including antibodies of any class or subclass, including IgG and sub-classes thereof, IgM, IgE, IgA, and IgD. The antigen-binding domain of a CAR can contain complementary determining regions (CDR) of an antibody, variable regions of an antibody, and/or antigen binding fragments thereof. For example, the antigen-binding domain for a CD19 CAR can be derived from a human monoclonal antibody to CD19, such as those described in U.S. Pat. No. 7,109,304, for use in accordance with the disclosed compositions and methods. In some forms, the antigen-binding domain can include an F(ab′)2, Fab′, Fab, Fv or scFv.

The CAR can contain a spacer domain (also referred to as hinge domain) that is located between the extracellular antigen-binding domain and the transmembrane domain. A spacer domain is an amino acid segment that is generally found between two domains of a protein and may allow for flexibility of the protein and movement of one or both of the domains relative to one another. Any amino acid sequence that provides such flexibility and movement of the extracellular antigen-binding domain relative to the transmembrane domain can be used. The spacer domain can be a spacer or hinge domain of a naturally occurring protein. In some forms, the hinge domain is derived from CD8a, such as, a portion of the hinge domain of CD8a, e.g., a fragment containing at least 5 (e.g., 5, 10, 15, 20, 25, 30, 35, or 40) consecutive amino acids of the hinge domain of CD8a. Hinge domains of antibodies, such as an IgG, IgA, IgM, IgE, or IgD antibodies can also be used. In some forms, the hinge domain is the hinge domain that joins the constant CH1 and CH2 domains of an antibody. Non-naturally occurring peptides may also be used as spacer domains. For example, the spacer domain can be a peptide linker, such as a (G×S)n linker, wherein x and n, independently can be an integer of 3 or more, including 3, 4, 5, 6, 7, 8, 9, 10, 11, 12, or more.

The CARs can contain a transmembrane domain that can be directly or indirectly fused to the antigen-binding domain. The transmembrane domain may be derived either from a natural or a synthetic source. As used herein, a “transmembrane domain” refers to any protein structure that is thermodynamically stable in a cell membrane, preferably a eukaryotic cell membrane. In some forms, the transmembrane domain of the CAR includes a transmembrane domain of an alpha, beta or zeta chain of a T-cell receptor, CD8, CD4, CD28, CD137, CD80, CD86, CD152 or PD1, or a portion thereof. Transmembrane domains can also contain at least a portion of a synthetic, non-naturally occurring protein segment. In some forms, the transmembrane domain is a synthetic, non-naturally occurring alpha helix or beta sheet. In some forms, the protein segment is at least about 15 amino acids, e.g., at least 15, 16, 17, 18, 19, 20, 21, 22, 23, 24, 25, 26, 27, 28, 29, 30, or more amino acids. Examples of synthetic transmembrane domains are known in the art, for example in U.S. Pat. No. 7,052,906 and PCT Publication No. WO 2000/032776.

The intracellular signaling domain is responsible for activation of at least one of the normal effector functions of the immune effector cell expressing the CAR. The term effector function refers to a specialized function of a cell. Effector function of a T cell, for example, may be cytolytic activity or helper activity including the secretion of cytokines. In some forms, an intracellular signaling domain includes the zeta chain of the T cell receptor or any of its homologs (e.g., eta, delta, gamma or epsilon), MB1 chain, B29, Fc RIII, Fc RI and combinations of signaling molecules such as CD3ζ and CD28, 4-1BB, OX40 and combination thereof, as well as other similar molecules and fragments. Intracellular signaling portions of other members of the families of activating proteins can be used, such as FcγRIII and FcεRI.

Many immune effector cells require co-stimulation, in addition to stimulation of an antigen-specific signal, to promote cell proliferation, differentiation and survival, as well as to activate effector functions of the cell. In some forms, the CAR can contain at least one co-stimulatory signaling domain. The term co-stimulatory signaling domain, refers to at least a portion of a protein that mediates signal transduction within a cell to induce an immune response such as an effector function. The co-stimulatory signaling domain can be a cytoplasmic signaling domain from a co-stimulatory protein, which transduces a signal and modulates responses mediated by immune cells, such as T cells, NK cells, macrophages, neutrophils, or eosinophils. In some forms, the co-stimulatory signaling domain is derived from a co-stimulatory molecule selected from the group consisting of CD27, CD28, CD137, 0X40, CD30, CD40, CD3, LFA-1, ICOS, CD2, CD7, LIGHT, NKG2C, B7-H3, ligands of CD83 and combinations thereof.

CARs can be used in order to generate immunoresponsive cells, such as T cells, specific for selected targets, such as malignant cells, with a wide variety of receptor chimera constructs having been described (see U.S. Pat. Nos. 5,843,728; 5,851,828; 5,912,170; 6,004,811; 6,284,240; 6,392,013; 6,410,014; 6,753,162; 8,211,422; and, PCT Publication WO9215322). Alternative CAR constructs can be characterized as belonging to successive generations. First-generation CARs typically consist of a single-chain variable fragment of an antibody specific for an antigen, for example comprising a VL linked to a VH of a specific antibody, linked by a flexible linker, for example by a CD8a hinge domain and a CD8a transmembrane domain, to the transmembrane and intracellular signaling domains of either CD3ζ or FcRγ (scFv-CD3ζ or scFv-FcRγ; see U.S. Pat. Nos. 7,741,465; 5,912,172; 5,906,936). Second-generation CARs incorporate the intracellular domains of one or more costimulatory molecules, such as CD28, OX40 (CD134), or 4-1BB (CD137) within the endodomain (for example scFv-CD28/OX40/4-1BB-CD3ζ; see U.S. Pat. Nos. 8,911,993; 8,916,381; 8,975,071; 9,101,584; 9,102,760; 9,102,761). Third-generation CARs include a combination of costimulatory endodomains, such a CD3ζ-chain, CD97, GDI 1a-CD18, CD2, ICOS, CD27, CD154, CDS, OX40, 4-1BB, or CD28 signaling domains (for example scFv-CD28-4-1BB-CD3ζ or scFv-CD28-OX40-CD3ζ; see U.S. Pat. Nos. 8,906,682; 8,399,645; 5,686,281; PCT Publication No. WO2014134165; PCT Publication No. WO2012079000). Alternatively, costimulation can be orchestrated by expressing CARs in antigen-specific T cells, chosen so as to be activated and expanded following engagement of their native αβTCR, for example by antigen on professional antigen-presenting cells, with attendant costimulation. Any of the first, second, or third generation CARs described above can be used in accordance with the disclosed compositions and methods.

In some forms, the HDR template can encode a CAR targeting one or more antigens specific for cancer, an inflammatory disease, a neuronal disorder, HIV/AIDS, diabetes, a cardiovascular disease, an infectious disease, an autoimmune disease, or combinations thereof. One of skill in the art, based on general knowledge in the field and/or routine experimentation would be able to determine the appropriate antigen to be targeted by a CAR for a specific disease, disorder or condition.

Exemplary antigens specific for cancer that could be targeted by the CAR include, but are not limited to, 4-1BB, 5T4, adenocarcinoma antigen, alpha-fetoprotein, BAFF, B-lymphoma cell, C242 antigen, CA-125, carbonic anhydrase 9 (CA-IX), C-MET, CCR4, CD 152, CD 19, CD20, CD200, CD22, CD221, CD23 (IgE receptor), CD28, CD30 (TNFRSF8), CD33, CD4, CD40, CD44 v6, CD51, CD52, CD56, CD74, CD80, CEA, CNT0888, CTLA-4, DRS, EGFR, EpCAM, CD3, FAP, fibronectin extra domain-B, folate receptor 1, GD2, GD3 ganglioside, glycoprotein 75, GPNMB, HER2/neu, HGF, human scatter factor receptor kinase, IGF-1 receptor, IGF-I, IgG1, L1-CAM, IL-13, IL-6, insulin-like growth factor I receptor, integrin α5β1, integrin αvβ3, MORAb-009, MS4A1, MUC1, mucin CanAg, N-glycolylneuraminic acid, NPC-1C, PDGF-R a, PDL192, phosphatidylserine, prostatic carcinoma cells, RANKL, RON, ROR1, SCH 900105, SDC1, SLAMF7, TAG-72, tenascin C, TGF beta 2, TGF-β, TRAIL-R1, TRAIL-R2, tumor antigen CTAA16.88, VEGF-A, VEGFR-1, VEGFR2, vimentin, and combinations thereof.

Exemplary antigens specific for an inflammatory disease that could be targeted by the CAR include, but are not limited to, AOC3 (VAP-1), CAM-3001, CCL11 (eotaxin-1), CD 125, CD 147 (basigin), CD 154 (CD40L), CD2, CD20, CD23 (IgE receptor), CD25 (a chain of IL-2 receptor), CD3, CD4, CD5, IFN-α, IFN-γ, IgE, IgE Fc region, IL-1, IL-12, IL-23, IL-13, IL-17, IL-17A, IL-22, IL-4, IL-5, IL-5, IL-6, IL-6 receptor, integrin a4, integrin α4β7, Lama glama, LFA-1 (CD 11a), MEDI-528, myostatin, OX-40, rhuMAb β7, scleroscin, SOST, TGF beta 1, TNF-a, VEGF-A, and combinations thereof.

Exemplary antigens specific for a neuronal disorder that could be targeted by the CAR include, but are not limited to, beta amyloid, MABT5102A, and combinations thereof.

Exemplary antigens specific for diabetes that could be targeted by the CAR include, but are not limited to, L-I β, CD3, and combinations thereof.

Exemplary antigens specific for a cardiovascular disease that could be targeted by the CAR include, but are not limited to, C5, cardiac myosin, CD41 (integrin alpha-lib), fibrin II, beta chain, ITGB2 (CD 18), sphingosine-1-phosphate, and combinations thereof.

Exemplary antigens specific for an infectious disease that could be targeted by the CAR include, but are not limited to, anthrax toxin, CCR5, CD4, clumping factor A, cytomegalovirus, cytomegalovirus glycoprotein B, endotoxin, Escherichia coli, hepatitis B surface antigen, hepatitis B virus, HIV-1, Hsp90, Influenza A hemagglutinin, lipoteichoic acid, Pseudomonas aeruginosa, rabies virus glycoprotein, respiratory syncytial virus, TNF-a, and combinations thereof.

In preferred forms, the CAR targets one or more antigens selected from an antigen listed in Table 3.

TABLE 3
Non-limiting examples of CAR targets

| | | | | | |
|---|---|---|---|---|---|
| AFP | AKAP-4 | ALK | Androgen | B7H3 | BCMA |
| | | | receptor | | |
| Bcr-Abl | BORIS | Carbonic | CD123 | CD138 | CD174 |
| CD19 | CD20 | CD22 | CD30 | CD33 | CD38 |
| CD80 | CD86 | CEA | CEACAM5 | CEACAM6 | Cyclin |
| CYP1B1 | EBV | EGFR | EGFR806 | EGFRvIII | EpCAM |
| EpCAM | EphA2 | ERG | ETV6-AML | FAP | Fos-related |
| | | | | | antigen1 |
| Fucosyl | fusion | GD2 | GD3 | GloboH | GM3 |
| gp100 | GPC3 | HER- | HER2 | HMWMAA | HPV E6/E7 |
| | | 2/neu | | | |
| hTERT | Idiotype | IL12 | IL13RA2 | IM19 | IX |
| LCK | Legumain | lgK | LMP2 | MAD-CT-1 | MAD-CT-2 |
| MAGE | MelanA/MART1 | Mesothelin | MET | ML-IAP | MUC1 |
| Mutant | MYCN | NA17 | NKG2D-L | NY-BR-1 | NY-ESO-1 |
| p53 | | | | | |
| NY-ESO- | OY-TES1 | p53 | Page4 | PAP | PAX3 |
| 1 | | | | | |
| PAX5 | PD-L1 | PDGFR-β | PLAC1 | Polysialic | Proteinase3 |
| | | | | acid | (PR1) |
| PSA | PSCA | PSMA | Ras mutant | RGS5 | RhoC |
| ROR1 | SART3 | sLe(a) | Sperm protein | SSX2 | STn |
| | | | 17 | | |
| Survivin | Tie2 | Tn | TRP-2 | Tyrosinase | VEGFR2 |
| WT1 | XAGE | | | | |

Preferably, the CAR can be an anti-CD19 CAR (e.g., CD19BBz) or an anti-CD22 CAR (e.g., CD22BBz). In some forms, the CAR can be bispecific. In some forms, the CAR can be multivalent.

Bispecific or multi-specific (multivalent) CARs, e.g., including, but not limited to, CARs described in WO 2014/4011988 and US20150038684, are contemplated for use in the disclosed methods and compositions.

B. Cells to be Modified

The disclosed gene editing compositions and methods can be used to achieve genomic modification of any cell type. For example, the cell can be a prokaryotic cell or a eukaryotic cell. The cell can be a mammalian cell. The mammalian cell many be a non-human mammal, e.g., primate, bovine, ovine, porcine, canine, rodent, Leporidae such as monkey, cow, sheep, pig, dog, rabbit, rat or mouse cell. The cell can be a non-mammalian eukaryotic cell such as poultry bird (e.g., chicken), vertebrate fish (e.g., salmon) or shellfish (e.g., oyster, claim, lobster, shrimp) cell. The cell can also be a plant cell. The plant cell can be of a monocot or dicot or of a crop or grain plant such as cassava, corn, sorghum, soybean, wheat, oat or rice. The plant cell can also be of an algae, tree or production plant, fruit or vegetable (e.g., trees such as citrus trees, e.g., orange, grapefruit or lemon trees; peach or nectarine trees; apple or pear trees; nut trees such as almond or walnut or pistachio trees; nightshade plants; plants of the genus Brassica; plants of the genus Lactuca; plants of the genus Spinacia; plants of the genus Capsicum; cotton, tobacco, asparagus, carrot, cabbage, broccoli, cauliflower, tomato, eggplant, pepper, lettuce, spinach, strawberry, blueberry, raspberry, blackberry, grape, coffee, cocoa, etc.)

In preferred forms, the cell to be modified is a human cell including, but not limited to, skin cells, lung cells, heart cells, kidney cells, pancreatic cells, muscle cells, neuronal cells, human embryonic stem cells, and pluripotent stem cells. More preferably, the cell to be modified can be a T cell (e.g., CD8+ T cells such as effector T cells, memory T cells, central memory T cells, and effector memory T cells, or CD4+ T cells such as Th1 cells, Th2 cells, Th17 cells, and Treg cells), hematopoietic stem cell (HSC), macrophage, natural killer cell (NK), or dendritic cell (DC).

The cell can be from established cell lines or they can be primary cells, where “primary cells,” refers to cells and cells cultures that have been derived from a subject and allowed to grow in vitro for a limited number of passages, i.e. splittings, of the culture.

1. Sources of T Cells

Prior to expansion and genetic modification, T cells can be obtained from a diseased or healthy subject. T cells can be obtained from a number of samples, including peripheral blood mononuclear cells, bone marrow, lymph node tissue, cord blood, thymus tissue, tissue from a site of infection, ascites, pleural effusion, spleen tissue, and tumors. In some forms, T cells can be obtained from a unit of blood collected from a subject using any number of techniques known to the skilled artisan, such as Ficoll™ separation. In one preferred form, cells from the circulating blood of an individual are obtained by apheresis. The apheresis product typically contains lymphocytes, including T cells, monocytes, granulocytes, B cells, other nucleated white blood cells, red blood cells, and platelets. The cells collected by apheresis can be washed to remove the plasma fraction and to place the cells in an appropriate buffer or media for subsequent processing steps. In some forms, the cells are washed with phosphate buffered saline (PBS). In some forms, the wash solution lacks calcium and can lack magnesium or can lack many if not all divalent cations. After washing, the cells can be resuspended in a variety of biocompatible buffers, such as, for example, Ca2+-free, Mg2+-free PBS, PlasmaLyte A, or other saline solution with or without buffer. Alternatively, the undesirable components of the apheresis sample can be removed and the cells directly resuspended in culture media.

In some forms, T cells can be isolated from peripheral blood lymphocytes by lysing the red blood cells and depleting the monocytes, for example, by centrifugation through a PERCOLL™ gradient or by counterflow centrifugal elutriation. A specific subpopulation of T cells, such as CD3+, CD28+, CD4+, CD8+, CD45RA+, and CD45RO+ T cells, can be further isolated by positive or negative selection techniques. For example, in some forms, T cells can be isolated by incubation with anti-CD3/anti-CD28 (i.e., 3×28)-conjugated beads, such as DYNABEADS® M-450 CD3/CD28 T, for a time period sufficient for positive selection of the desired T cells.

C. Pharmaceutical Compositions

Disclosed are pharmaceutical compositions containing a genetically modified cell or a population of genetically modified cells with a pharmaceutically acceptable buffer, carrier, diluent or excipient. The population of cells can be derived by expanding an isolated genetically modified cell (e.g., CAR T cell), e.g., a homogenous population. In some forms, the population of cells can contain variable or different genetically modified cells, e.g., a heterogeneous population. The cells can be modified to be bispecific or multispecific. The cell can have been isolated from a diseased or healthy subject prior to genetic modification. Introduction of gene editing compositions (e.g., RNA-guided endonuclease and the one or more AAV vectors) to the cell can be performed ex vivo.

“Pharmaceutically acceptable carrier” describes a pharmaceutically acceptable material, composition, or vehicle that is involved in carrying or transporting a compound of interest from one tissue, organ, or portion of the body to another tissue, organ, or portion of the body. For example, the carrier can be a liquid or solid filler, diluent, excipient, solvent, or encapsulating material, or a combination thereof. Each component of the carrier must be “pharmaceutically acceptable” in that it must be compatible with the other ingredients of the formulation. It must also be suitable for use in contact with any tissues or organs with which it may come in contact, meaning that it must not carry a risk of toxicity, irritation, allergic response, immunogenicity, or any other complication that excessively outweighs its therapeutic benefits.

Such pharmaceutical compositions can comprise buffers such as neutral buffered saline, phosphate buffered saline and the like; carbohydrates such as glucose, mannose, sucrose or dextrans, mannitol; proteins; polypeptides or amino acids such as glycine; antioxidants; chelating agents such as EDTA or glutathione; adjuvants (e.g., aluminum hydroxide); and preservatives.

The pharmaceutical compositions can be formulated for delivery via any route of administration. “Route of administration” can refer to any administration pathway known in the art, including but not limited to aerosol, nasal, oral, intravenous, intramuscular, intraperitoneal, inhalation, transmucosal, transdermal, parenteral, implantable pump, continuous infusion, topical application, capsules and/or injections. The pharmaceutical compositions are preferably formulated for intravenous administration.

The disclosed pharmaceutical compositions can be administered in a manner appropriate to a disease to be treated (or prevented). The quantity and frequency of administration will be determined by such factors as the condition of the patient, and the type and severity of the patient's disease, although appropriate dosages can be determined by clinical trials.

The disclosed pharmaceutical compositions can be delivered in a therapeutically effective amount. The precise therapeutically effective amount is that amount of the composition that will yield the most effective results in terms of efficacy of treatment in a given subject. This amount will vary depending upon a variety of factors, including but not limited to the characteristics of the therapeutic compound (including activity, pharmacokinetics, pharmacodynamics, and bioavailability), the physiological condition of the subject (including age, sex, disease type and stage, general physical condition, responsiveness to a given dosage, and type of medication), the nature of the pharmaceutically acceptable carrier or carriers in the formulation, and the route of administration. One skilled in the clinical and pharmacological arts will be able to determine a therapeutically effective amount through routine experimentation, for instance, by monitoring a subject's response to administration of a compound and adjusting the dosage accordingly. For additional guidance, see Remington: The Science and Practice of Pharmacy (Gennaro ed. 20th edition, Williams & Wilkins PA, USA) (2000).

### III. Methods of CAR-T Generation with CRISPR/Cpf1 and AAV Systems

Chimeric antigen receptor (CAR) T cells have recently become powerful players in the arsenal of immune-based cancer therapy. More recently, gene-editing technologies have enabled more direct engineering of immune cells. However, current lentiviral, retroviral, or CRISPR/Cas9 based methods have various limitations in CAR targeting efficiency and modularity, especially for generation of multi-component CAR T cells. Therefore, methods for cellular genome engineering that permit simple, efficient, and versatile permutations of combinatorial or simultaneous knockout and knock-in genomic modifications are provided. In particular, the AAV-Cpf1 KIKO method which uses a combination of viral and non-viral approaches to generate a stable CAR-T with homology-directed repair (HDR) knock-in and immune checkpoint knockout at high efficiency in one step is provided.

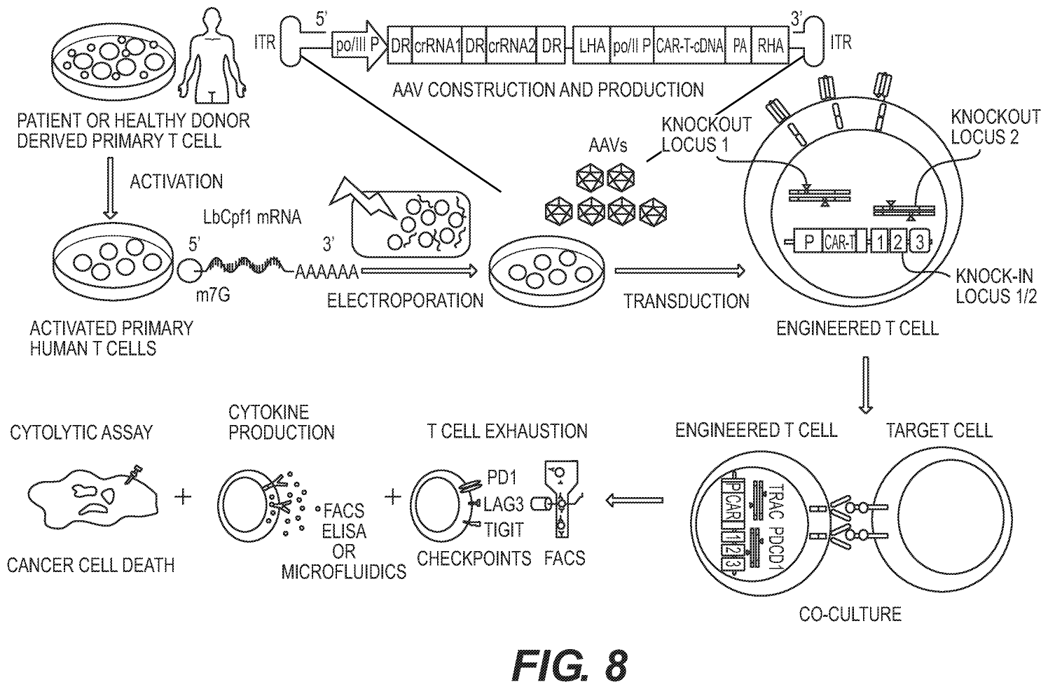

Advantages of this AAV-Cpf1 KIKO method include, but are not limited to, design simplicity, higher delivery efficiency, lower toxicity, reduced exhaustion, increased effector function, and long term CAR enrichment (e.g., compared to standard approaches such as lentiviral CRISPR/Cas9 based approaches). The efficiency of this approach makes it readily feasible to produce single knock-in, double knock-in, or three or more knock-in CAR-T cells on the order of 10⁸ to 10⁹ from a regular source of blood in two to three weeks, which is the scale and timeline typically needed in the clinical setting. FIG. 8 illustrates a simple workflow for the generation and functional testing of CAR-T cells using the AAV-Cpf1 KIKO system. This system can be used by, for example, both the scientific and clinical community for CAR-T research and production.

Gene editing compositions can be introduced to the cells together in the same or different composition, or the gene editing compositions can be introduced to cells separately. For example, in some forms, cells can be introduced to an RNA-guided endonuclease, followed by a vector (e.g., AAV vector) containing a sequence (e.g., a crRNA array) that encodes one or more crRNAs and optionally, one or more HDR templates and/or sequences homologous to one or more target sites. Alternatively, in some forms, cells can be first introduced to a vector (e.g., AAV vector) containing a sequence (e.g., a crRNA array) that encodes one or more crRNAs and optionally, one or more HDR templates and/or sequences homologous to one or more target sites, followed by an RNA-guided endonuclease. In some forms, an RNA-guided endonuclease and a vector (e.g., AAV vector) containing a sequence (e.g., a crRNA array) that encodes one or more crRNAs and optionally, one or more HDR templates and/or sequences homologous to one or more target sites are introduced to the cells simultaneously (e.g., in the same or different composition).

The following provides example materials and protocols that can be used to implement and use the disclosed systems.

A. Materials

1. Plasmids & DNA
(i) NSL-LbCpf1-NSL mRNA (TriLink BioTechnologies)
   Modified mRNA transcript with full substitution of pseudo-U and Capped (Cap 1) using CleanCap™ AG. mRNA can be polyadenylated with DNase and phosphatase treatment. mRNA can be purified by silica membrane and packaged as a solution in 1 mM Sodium Citrate, pH 6.4.
(ii) Plasmids: AAV6/AAV9, PDF6, AAV vector including pXD017, pXD017-39, pXD040, pXD042, pXD043, pXD050, pXD053, and pXD054

2. Cell Lines
(i) Human peripheral blood CD4+ T cells (STEMCELL Technologies, or other donors)
(ii) HEK293FT cells (ThermoFisher)
(iii) NALM6 cells (ATCC)

3. Kits & Chemicals
(i) X-VIVO 15, serum free hematopoietic cell medium (Lonza)
(ii) CD3/CD28 Dynabeads (Thermo Fisher)
(iii) Polyethyleneimine (Sigma)
(iv) Pierce Universal Nuclease (Thermo Fisher)
(v) QuickExtract DNA Extraction Solution (Epicentre)
(vi) Taqman assays (ThermoFisher)
(vii) T7E1 (New England BioLabs)
(viii) CD22-Fc (R&D system)
(ix) APC-CD4-Clone A161A1Biolegend-357408
   FITC-CD3-Clone HIT3a-Biolegend-300306
   PE-IgG-Fc-Clone HP6017-Biolegend-409304
   PD-1-FITC-Clone EH12.2H7-Biolegend-329904
   TIGIT-APC-Clone A15153G-Biolegend-372705
   LAGS-Percp/cy5.5-Clone 11C3C65-Biolegend-369312
   APC-anti-DYKDDDDK Tag-Clone L5-Biolegend-637308
   PerCP/Cyanine5.5 anti-DYKDDDDK Tag-Clone L5-Biolegend-637326
   IFNγ-APC-Clone 4S.B3-Biolegend-502512
TNF-α-FITC-Clone MAb11-Biolegend-502906
(x) Nextera Amplicon Tagment Mix (Illumina)
(xi) E-Gel™ X Agarose Gels, 2% (ThermoFisher)
(xii) Gibson assembly master mix (New England Biolabs)
(xiii) Quick ligation kit (New England Biolabs)
(ixv) Neon electroporation 100 μL kit and 10 μL kit (ThermoFisher)
(xv) IL-2 (Biolegend)
(xvi) Human AB serum (Corning)
(xvii) Fixation/Permeabilization Solution Kit (BD)
(xviii) XenoLight D-Luciferin—K+ Salt Bioluminescent Substrate (PerkinElmer)
(ixx) QIAquick gel extraction kit (QIAGEN)
(xx) Phusion flash high-fidelity PCR master mix (ThermoFisher)
(xxi) Amicon ultra centrifugal filter 100 kDa (Millipore)
(xxii) PEG 8000 powder (Promega)
(xxiii) E-Gel™ Low Range Quantitative DNA Ladder (ThermoFisher).

B. Equipment
(i) PCR Thermocycler
(ii) Tissue culture hood
(iii) 15-cm tissue culture dishes (Corning)
(iv) Retronectin-coated plates (Takara)
(v) Neon® Transfection System (ThermoFisher)
(vi) Bioanalyzer (Agilent)
(vii) Pipettes and tips
(viii) Next generation sequencing machines (Illumina)
(ix) Cell culture incubators (37° C., 5% CO₂)
(x) Countess automated cell counter (Thermo Fisher)
(xi) Plate reader (PerkinElmer)
(xii) BD FACSAria II (BD Biosciences)
(xiii) FlowJo software 9.9.4 (Treestar, Ashland, Oreg.)

C. Construction of AAV Vectors

1. crRNA Expression Vector Design and Construction
(i) Identify genes for knockout by targeted delivery of HDR template. Here, TRAC and PDCD1 are used as examples, but note that any gene with a Cpf1 PAM sequence can be targeted.
(ii) Design LbCpf1 crRNA (20 bp) with Benchling or other computational pipelines.

crTRAC:
(SEQ ID NO: 1)
GAGTCTCTCAGCTGGTACAC
crPDCD1:
(SEQ ID NO: 2)
GCACGAAGCTCTCCGATGTG

(iii) Synthesize oligonucleotides with two LbCpf1 direct repeats and sticky ends.
(iv) Digest pXD017 with FD BbsI and insert guide after U6 promoter (pXD017-39).

2. CAR Sequence Generation
(i) Generation of CD22BBz CAR can be performed as previously described (Haso, W., et al., Blood., 121(7):1165-74 (2013). CD22 binding scFV (m971) specific for the human CD22 followed by CD8 hinge-transmembrane-regions linked to 4-1BB (CD137) intracellular domains and CD3ζ intracellular domain.
(ii) The sequence of CD19 binding scFv (FMC63) can be found from NCBI (GenBank: HM852952) and can be followed by CD8 hinge-transmembrane-regions linked to 4-1BB (CD137) intracellular domains and CD3ζ intracellular domain (Kochenderfer, J N., et al., J. Immunother., 32(7):689-702 (2009)). In order to detect CD19BBz CAR in different way, the Flag-tag sequence (GATTACAAAGACGATGACGATAAG; SEQ ID NO:3) can be added after the CD8α leader sequence.
(iii) Synthesize m971-BBz and FMC63-BBz using gBlock (IDT).

3. HDR Template Design
(i) Amplify left and right homologous arms of the TRAC or PDCD1 locus from primary CD4+ T cells by PCR using locus-specific primer sets with multiple cloning site (MCS). PCR annealing temperature (60° C.).

TRAC_HDR_F1 (With AAV vector overlap sequence)
(SEQ ID NO: 4)
TCAACTAGATCTTGAGACAAGGTACGATGTAAGGAGCT
GCTGTGACT
TRAC_HDR_R1 (With MCS)
(SEQ ID NO: 5)
GGTACCTCGAGCGTACGGGTCAGGGTTCTGGATATCTGT
G
TRAC_HDR_F2 (With MCS)
(SEQ ID NO: 6)
CGTACGCTCGAGGTACCGAGAGACTCTAAATCCAGTGAC
AAG
TRAC_HDR_R2 (With AAV vector overlap sequence)
(SEQ ID NO: 7)
CTTTTATTAAGCTTGATATCGAATTGTGGGTTAATGAGT
GACTGCG
PDCD1_HDR_F1 (With AAV vector overlap sequence)
(SEQ ID NO: 8)
TGGCAGGAGAGGGCACGTGGGCAGCCTCACGTAGAAGG
AA
PDCD1_HDR_R1 (With MCS)
(SEQ ID NO: 9)
TCCGAGAATTCTTTGTTAACTGTGTTGGAGAAGCTGCAG
GT
PDCD1_HDR_F2 (With MCS)
(SEQ ID NO: 10)
CACAGTTAACAAAGAATTCTCGGAGAGCTTCGTGCTAAAC
TGG
PDCD1_HDR_R2 (With AAV vector overlap sequence)
(SEQ ID NO: 19)
GCGGCCGCTCGGTCCGCACCTGATCCTGTGCAGGAGGG

(ii) Sequence amplicons (e.g., Yale Keck or any other Sanger sequencing facility).

4. AAV-crRNA-HDR-CAR Vector Cloning
(i) pXD040 construction: Clone HDR sequences into the AAV vector (pXD017-39) by Gibson assembly. Incubate samples in a thermocycler at 50° C. for 30 minutes.
(ii) pXD043 (CD22CAR) and pXD054 (CD19CAR) construction: Digest pXD040 with BsiwI and Acc65I, and then clone CAR sequences into MCS by Gibson assembly.

D. AAV Production and Titration

1. AAV Production
(i) Transfect HEK293FT cells with AAV constructs in 15-cm tissue culture dishes, AAV2 transgene vectors, packaging (pDF6) plasmid, and AAV6/9 serotype plasmid together with polyethyleneimine (PEI).
(ii) Collect transfected cells with PBS after 72 hours of transfection.

2. AAV Purification and Titration
(i) Mix transfected cells with pure chloroform ( 1/10 volume).
(ii) Incubate cells at 37° C. with vigorous shaking for 1 hour.
(iii) Add NaCl to a final concentration of 1 M.
(iv) Centrifuge at 20,000 g at 4° C. for 15 minutes.
(v) Transfer aqueous layer to another tube and discard the chloroform layer.
(vi) Add PEG8000 to the sample until 10% (w/v) and shake until dissolved.
(vii) Incubate the mixture at 4° C. for 1 hour and then centrifuge at 20,000 g at 4° C.
(viii) Discard supernatant and suspend the pellet in DPBS with MgCl₂.
(ix) Treat the sample with universal nuclease and incubate at 37° C. for 30 minutes.
(x) Add chloroform (1:1 volume), shake and centrifuge at 12,000 g at 4° C. for 15 minutes.
(xi) Isolate the aqueous layer and concentrate through a 100-kDa MWCO. Important step: concentrate AAV at high concentration so the volume can be reduced when performing the infection, which can decrease the toxicity of AAV. AAV should be aliquoted and stored at −80° C.
(xii) Titer virus by qPCR using custom Taqman assays (ThermoFisher) targeted to promoter U6.

E. T Cell Electroporation

Human primary peripheral blood CD4+ T cells can be acquired from healthy donors (STEMCELL technologies). T cells can be cultured in X-VIVO media (Lonza) with 5% human AB serum and recombinant human IL-2 30 U/mL.
(i) Activate T cells with CD3/CD28 Dynabeads for 2 days prior to electroporation.
(ii) Use magnetic holder to remove Dynabeads.
(iii) Prepare cells at a density of 2×10⁵ cells per 10 μL tip reaction or 2×10⁶ cells per 100 μL tip reaction in electroporation Buffer R (Neon Transfection System Kits).
(iv) Mix with 1 μg or 10 μg of modified NLS-LbCpf1-NLS mRNA (TriLink) according to reaction volume.
(v) Electric shock at program 24 (1,600V, 10 ms and three pulses).
(vi) Transfer cells into 200 μl or 1 mL of pre-warmed X-VIVO media (without antibiotics) immediately after electroporation.
(vii) Add indicated volumes of AAV (AAV volume to not exceed 20% of culture volume) into the T cells 2-4 hours after electroporation. The CAR(s) will begin to be expressed after two to three days and have enrichment after stimulation with target cells.

F. CAR-T Detection by Flow Cytometry
(i) After electroporation for 5 days, incubate 1×10⁶ CD22BBz CAR transduced T cells with 0.2 μg CD22-Fc (R&D system) in 100 μL PBS for 30 minutes, and then stain with PE-IgG-Fc and FITC-CD3 antibodies for 30 minutes.
(ii) For CD19CAR detection, incubate CD19BBz CAR transduced T cells with APC-anti-DYKDDDDK Tag (SEQ ID NO:11) and FITC-CD3 antibodies for 30 minutes.
(iii) Wash cells twice and quantify and sort labeled cells on BD FACSAria II.
(iv) The staining patterns can be analyzed using FlowJo software 9.9.4 (Treestar, Ashland, Oreg.).

G. T7E1 Assay

Five days after electroporation, harvest the bulk transduced T cells and sorted T cells. The genomic DNA can be collected using the QuickExtract DNA Extraction

Solution (Epicentre).
(i) PCR amplify target loci from genomic DNA around cut site.

TRAC_suvF:
(SEQ ID NO: 12)
CTGAGTCCCAGTCCATCACG
TRAC_suvR:
(SEQ ID NO: 13)
AGGGTTTTGGTGGCAATGG
PDCD1_suvF:
(SEQ ID NO: 14)
GTAGGTGCCGCTGTCATTGC
PDCD1_suvR:
(SEQ ID NO: 15)
GAGCAGTGCAGACAGGACCA

(ii) Run PCR amplicons on 2% E-gel EX and purify (with known band size) using QIAquick Gel Extraction Kit.
(iii) After purification, denature 200 ng of purified PCR product, anneal, and digest with T7E1, 37° C. 45 minutes (New England BioLabs).
(iv) Load digested PCR products into 2% E-gel EX and quantify DNA fragment abundance using E-Gel™ Low Range Quantitative DNA Ladder (ThermoFisher).

H. HDR Quantification and NGS Sequencing Analysis

1. Semi-Quantitative In-Out PCR
(i) Use three primers for In-Out PCR:
   TRAC 1st: binds to a sequence of the left TRAC homology arm
   TRAC 2nd: binds to genomic sequence outside of this AAV donor
   CD22CAR 3rd primer: recognizes a sequence contained in the m971-BBz cassette

TRAC 1st:
(SEQ ID NO: 16)
CCCTTGTCCATCACTGGCAT
TRAC 2nd:
(SEQ ID NO: 17)
GCACACCCCTCATCTGACTT
CD22CAR 3rd:
(SEQ ID NO: 18)
GAAATCAAAGCGGCCGCAG

(ii) Normalize amplicon (labeled TRAC-HDR) concentration by comparison to the product resulting from the uninfected control with genomic DNA isolated from human CD4+ T cells.
(iii) PCR products can be used for Nextera library preparation following the manufacturer's protocols (Illumina)
(iv) Prepped libraries can be sequenced on 100-bp single-end reads on an Illumina HiSeq 4000 instrument or equivalent.

2. Indel Quantification
(i) Some PCR products from amplification around cut site of genomic DNA (same samples as T7E1 assay) can be used for Nextera library preparation following the manufacturer's protocols (Illumina).
(ii) Prepped libraries can be sequenced on 100-bp paired-end reads on an Illumina HiSeq 4000 instrument or equivalent (generating 29 to 74 million reads per library).
(iii) Map paired reads to amplicon sequences (expected sequences provided in FASTA form to generate indices) using BWA-MEM with the -M option.
(iv) Discard 100 bp reads in SAM file that fall outside a +/−75 bp window of expected cut site within the amplicon.
(v) Discard soft-clipped reads (identified with “S” character in CIGAR string).
(vi) Identify indel reads by the presence of “I” or “D” characters within the CIGAR string.
(vii) Quantify cutting efficiency as percentage of indels over total (indel plus wild-type reads) within the defined window.

3. HDR Quantification
(i) Map reads to possible amplicons based on primer combinations and HDR status.
(ii) Define “informative” amplicons as truncated so that 100 bp reads would have at least 20 bp homology with the CAR sequence (or with the other TRAC arm, in the case of wild-type sequences). Informative reads can be used to distinguish wild-type, NHEJ and HDR reads with higher confidence.
(iii) Map paired reads to amplicon sequences using BWA-MEM with -M flag to generate SAM files.
(iv) Use SAMtools to convert SAM files to BAM, sort, index, and generate summary statistics of read counts with the idxstats option.
(v) To quantify wild-type versus NHEJ reads, take reads that mapped to “info_nonHDR” sequence (described below), and call reads with indels (“I” or “D” characters within the CIGAR string) as NHEJ. Otherwise call reads as wild-type.
(vi) Pool read counts for downstream analysis.
(vii) Schema for amplicon sequences and quantifications provided below:
   amplicon_nonHDR: refers to full amplicon from F1 and R1 of genomic, wild-type DNA.
   amplicon_CAR_F1: refers to full amplicon from F1 and R1 of expected, integrated CAR.
   amplicon_CAR_F2: refers to full amplicon from F2 (primer site within the CAR as opposed to outside) and R1 of expected, integrated CAR.
   info_nonHDR: same as amplicon_nonHDR, except truncated to 80 bp of the TRAC arms.
   info_CAR_F1: same as amplicon_CAR_F1, except truncated to 80 bp of the TRAC arms flanking the TRAC-CAR interface.
   info_CAR_F2: same as amplicon_CAR_F2, except truncated to 80 bp of the TRAC arms flanking the TRAC-CAR interface (relevant to the right arm only, since F2 is within the CAR sequence).
   HDR, NHEJ, and WT scores were calculated as follows:
      info_nonHDR=info_WT+info_NHEJ
      hdr_score=info_CAR_F2/(info_CAR_F2+info_nonHDR)
      wt_score=info_WT/(info_CAR_F2+info_nonHDR)
      nhej_score=info_NHEJ/(info_CAR_F2+info_nonHDR)

I. Co-Culture Functional Assays

1. Stable Cell Line Generation
(i) Generate lentivirus including GFP-Luciferase reporter genes.
(ii) Infect NALM6 cells (ATCC) with 2× concentrated lentivirus by spinoculation in retronectin-coated (Takara) plates at 800 g for 45 minutes at 32° C.
(iii) After infection for 2 days, sort GFP positive cells (NALM6-GL) by flow cytometry.
(iv) Perform a second round of sorting after culturing for an additional two days.
(v) Incubate cells with 150 μg/l D-Luciferin (PerkinElmer) and measure bioluminescence signal intensity by an IVIS system to assess luciferase expression.

2. Cancer Cell Cytolytic Assay (Kill Assay)
(i) Seed 2×10⁴ NALM6-GL cells in a 96 well plate.
(ii) Co-culture modified T cells with NALM6-GL at indicated E:T ratios for 24 hours.
(iii) Add 150 μg/ml D-Luciferin (PerkinElmer) into each well and measure luciferase assay intensity by a plate reader (PerkinElmer) to assess cell proliferation.

3. T Cell Exhaustion Assay
(i) Co-culture T cells modified by AAV with NALM6-GL cells at 0.5:1 E:T ratio for 24 hours.
(ii) Collect cells and wash once by DPBS. Incubate cells with 0.2 μg CD22-Fc (R&D Systems) in 100 μL DPBS for 30 minutes.
(iii) Stain cells with PE-IgG-Fc, PD-1-FITC, TIGIT-APC and LAG3-Percp/cy5.5 (Biolegend) for 30 minutes.
(iv) Measure stained cells by flow cytometry.

4. Intracellular Staining of IFNγ and TNF-α
(i) After infection for 5 days, co-culture AAV transduced CD22BBz CAR-T cells with NALM6 at 1:1 E:T ratio in fresh media supplemented with brefeldin A and 2 ng/mL IL-2.
(ii) After 5 hours of incubation, collect and stain for surface CAR.
(iii) Fix and permeabilize cells by fixation/permeabilization solution (BD) and add anti-IFNγ-APC or anti-TNF-α-FITC for intracellular staining.
(iv) After 30 minutes, wash stained cells by BD Perm/Wash™ buffer and measure cells by flow cytometry.

J. Time Taken
AAV construction: 1-2 weeks
AAV production and purification: 4-5 days
T cell electroporation and infection: 5 hours
CAR-T expression and detection: 5 days
Cytolytic assay: 24 hours
T cell exhaustion assay: 24 hours
T cell intracellular staining: 8 hours

### IV. Methods of Treatment

Disclosed herein are methods of treatment. An exemplary method involves treating a subject (e.g., a human) having a disease, disorder, or condition by administering to the subject an effective amount of the aforementioned pharmaceutical composition. Disclosed is a method of treating a subject having a disease, disorder, or condition associated with an elevated expression or specific expression of an antigen by administering to the subject an effective amount of a T cell modified according to the disclosed methods to contain a CAR that targets the antigen.

Further disclosed is a method of treating a subject having a disease, disorder, or condition by administering to the subject an effective amount of a pharmaceutical composition having a genetically modified cell, where the cell is modified by introducing to the cell: (a) an RNA-guided endonuclease; and (b) one or more AAV vectors including (i) a sequence encoding one or more crRNAs that direct the RNA-guided endonuclease to one or more target genes; and (ii) one or more HDR templates containing a sequence that encodes one or more chimeric antigen receptors (CAR); and (iii) one or more sequences homologous to a target site.

The cell can have been isolated from the subject having the disease, disorder, or condition, or from a healthy donor, prior to genetic modification.

A. Diseases to be Treated

The subject to be treated can have a disease, disorder, or condition such as but not limited to, cancer, an inflammatory disease, a neuronal disorder, HIV/AIDS, diabetes, a cardiovascular disease, an infectious disease, an immune system disorder such autoimmune disease, or combinations thereof. The disease, disorder, or condition can be associated with an elevated expression or specific expression of an antigen.

1. Cancers

Cancer is a disease of genetic instability, allowing a cancer cell to acquire the hallmarks proposed by Hanahan and Weinberg, including (i) self-sufficiency in growth signals; (ii) insensitivity to anti-growth signals; (iii) evading apoptosis; (iv) sustained angiogenesis; (v) tissue invasion and metastasis; (vi) limitless replicative potential; (vii) reprogramming of energy metabolism; and (viii) evading immune destruction (Cell., 144:646-674, (2011)).

Tumors, which can be treated in accordance with the disclosed methods, are classified according to the embryonic origin of the tissue from which the tumor is derived. Carcinomas are tumors arising from endodermal or ectodermal tissues such as skin or the epithelial lining of internal organs and glands. Sarcomas, which arise less frequently, are derived from mesodermal connective tissues such as bone, fat, and cartilage. The leukemias and lymphomas are malignant tumors of hematopoietic cells of the bone marrow. Leukemias proliferate as single cells, whereas lymphomas tend to grow as tumor masses. Malignant tumors may show up at numerous organs or tissues of the body to establish a cancer.

Table 4 provides a non-limiting list of cancers for which the CAR of the disclosed methods and compositions can target a specific or an associated antigen.

TABLE 4

| | | | | |
|---|---|---|---|---|
| Acute | Acute | Adrenocortical | AIDS-Related | Kaposi |
| Lymphoblastic | Myeloid | Carcinoma | Cancers | Sarcoma |
| Leukemia | Leukemia | | | |
| (ALL) | (AML) | | | |
| AIDS-Related | Primary CNS | Anal Cancer | Appendix Cancer | Astrocytomas |
| Lymphoma | Lymphoma | | (Gastrointestinal | |
| | | | Carcinoid | |
| | | | Tumors) | |
| Atypical | Brain Cancer | Basal Cell | Bile Duct Cancer | Bladder Cancer |
| Teratoid/ | | Carcinoma of the | | |
| Rhabdoid | | Skin | | |
| Tumor | | | | |
| Bone Cancer | Brain Tumors | Breast Cancer | Bronchial Tumors | Burkitt |
| (includes | | | | Lymphoma |
| Ewing Sarcoma | | | | |
| and | | | | |
| Osteosarcoma | | | | |
| and Malignant | | | | |
| Fibrous | | | | |
| Histiocytoma) | | | | |
| Non-Hodgkin | Carcinoid | Carcinoma of | Cardiac (Heart) | Embryonal |
| Lymphoma | Tumors | Unknown Primary | Tumors | Tumors |
| Germ Cell | Primary CNS | Cervical Cancer | Cholangio- | Chordoma |
| Tumor | Lymphoma | | carcinoma | |
| Chronic | Chronic | Chronic | Colorectal Cancer | Cranio- |
| Lymphocytic | Myelogenous | Myeloproliferative | | pharyngioma |
| Leukemia | Leukemia | Neoplasms | | |
| (CLL) | (CML) | | | |
| Cutaneous T- | Ductal | Endometrial | Ependymoma | Esophageal |
| Cell | Carcinoma In | Cancer | | Cancer |
| Lymphoma | Situ (DCIS) | | | |
| (Mycosis | | | | |
| Fungoides and | | | | |
| Sézary | | | | |
| Syndrome) | | | | |
| Esthesioneuro- | Ewing | Extracranial Germ | Eye Cancer | Intraocular |
| blastoma | Sarcoma | Cell Tumor | | Melanoma |
| Fallopian Tube | Fibrous | Osteosarcoma | Gallbladder | Gastric Cancer |
| Cancer | Histiocytoma | | Cancer | |
| | of Bone | | | |
| Stomach | Gastrointestinal | Gastrointestinal | Central Nervous | Extracranial |
| Cancer | Carcinoid | Stromal Tumors | System Germ Cell | Germ Cell |
| | Tumor | (GIST) | Tumors | Tumors |
| Extragonadal | Ovarian Germ | Testicular Cancer | Gestational | Hairy Cell |
| Germ Cell | Cell Tumors | | Trophoblastic | Leukemia |
| Tumors | | | Disease | |
| Head and Neck | Heart Tumors | Hepatocellular | Histiocytosis | Hodgkin |
| Cancer | | (Liver) Cancer | (Langerhans Cell) | Lymphoma |
| Hypopharyngeal | Intraocular | Islet Cell Tumors | Pancreatic | Kidney Cancer |
| Cancer | Melanoma | | Neuroendocrine | |
| | | | Tumors | |
| Renal Cell | Langerhans | Laryngeal Cancer | Leukemia | Lip and Oral |
| Cancer | Cell | | | Cavity Cancer |
| | Histiocytosis | | | |
| Liver Cancer | Lung Cancer | Lymphoma | Male Breast | Malignant |
| | (Non-Small | | Cancer | Fibrous |
| | Cell and | | | Histiocytoma |
| | Small Cell) | | | of Bone and |
| | | | | Osteosarcoma |
| Melanoma | Intraocular | Merkel Cell | Malignant | Metastatic |
| | (Eye) | Carcinoma (Skin | Mesothelioma | Cancer |
| | Melanoma | Cancer) | | |
| Metastatic | Midline Tract | Mouth Cancer | Multiple | Multiple |
| Squamous | Carcinoma | | Endocrine | Myeloma/Plasma |
| Neck Cancer | With NUT | | Neoplasia | Cell |
| with Occult | Gene Changes | | Syndromes | Neoplasms |
| Primary | | | | |
| Mycosis | Myelodysplastic | Myelodysplastic/ | Nasal Cavity and | Nasopharyngeal |
| Fungoides | Syndromes | Myeloproliferative | Paranasal Sinus | Cancer |
| (Lymphoma) | | Neoplasms | Cancer | |
| Neuroblastoma | Non-Small | Oral Cancer | and | Ovarian Cancer |
| | Cell Lung | | Oropharyngeal | |
| | Cancer | | Cancer | |
| Pancreatic | Papillomatosis | Paraganglioma | Paranasal Sinus | Parathyroid |
| Cancer | | | and Nasal Cavity | Cancer |
| | | | Cancer | |
| Penile Cancer | Pharyngeal | Pheochromocytoma | Pituitary Tumor | Plasma Cell |
| | Cancer | | | Neoplasm/Multiple |
| | | | | Myeloma |
| Pleuropulmonary | Primary | Primary Peritoneal | Prostate Cancer | Rectal Cancer |
| Blastoma | Central | Cancer | | |
| | Nervous | | | |
| | System (CNS) | | | |
| | Lymphoma | | | |
| Recurrent | Retinoblastoma | Rhabdomyosarcoma | Salivary Gland | Sarcoma |
| Cancer | | | Cancer | |
| Vascular | Uterine | Sézary Syndrome | Small Cell Lung | Small Intestine |
| Tumors | Sarcoma | (Lymphoma) | Cancer | Cancer |
| Soft Tissue | Squamous | Stomach (Gastric) | Throat Cancer | Thymoma |
| Sarcoma | Cell | Cancer | | |
| | Carcinoma | | | |
| Thymic | Thyroid | Transitional Cell | Carcinoma of | Ureter and |
| Carcinoma | Cancer | Cancer of the | Unknown Primary | Renal Pelvis |
| | | Renal Pelvis and | | |
| | | Ureter | | |
| Transitional | Urethral | Uterine Cancer | Vaginal Cancer | Vulvar Cancer |
| Cell Cancer | Cancer | | | |
| Wilms Tumor | | | | |

The disclosed compositions and methods can be used in the treatment of one or more cancers provided in Table 4.

The disclosed compositions and methods of treatment thereof are generally suited for treatment of carcinomas, sarcomas, lymphomas and leukemias. The described compositions and methods are useful for treating, or alleviating subjects having benign or malignant tumors by delaying or inhibiting the growth/proliferation or viability of tumor cells in a subject, reducing the number, growth or size of tumors, inhibiting or reducing metastasis of the tumor, and/or inhibiting or reducing symptoms associated with tumor development or growth.

The types of cancer that can be treated with the provided compositions and methods include, but are not limited to, cancers such as vascular cancer such as multiple myeloma, adenocarcinomas and sarcomas, of bone, bladder, brain, breast, cervical, colorectal, esophageal, kidney, liver, lung, nasopharangeal, pancreatic, prostate, skin, stomach, and uterine. In some forms, the compositions are used to treat multiple cancer types concurrently. The compositions can also be used to treat metastases or tumors at multiple locations.

Exemplary tumor cells include, but are not limited to, tumor cells of cancers, including leukemias including, but not limited to, acute leukemia, acute lymphocytic leukemia, acute myelocytic leukemias such as myeloblastic, promyelocytic, myelomonocytic, monocytic, erythroleukemia leukemias and myelodysplastic syndrome, chronic leukemias such as, but not limited to, chronic myelocytic (granulocytic) leukemia, chronic lymphocytic leukemia, hairy cell leukemia; polycythemia vera; lymphomas such as, but not limited to, Hodgkin's disease, non-Hodgkin's disease; multiple myelomas such as, but not limited to, smoldering multiple myeloma, nonsecretory myeloma, osteosclerotic myeloma, plasma cell leukemia, solitary plasmacytoma and extramedullary plasmacytoma; Waldenström's macroglobulinemia; monoclonal gammopathy of undetermined significance; benign monoclonal gammopathy; heavy chain disease; bone and connective tissue sarcomas such as, but not limited to, bone sarcoma, osteosarcoma, chondrosarcoma, Ewing's sarcoma, malignant giant cell tumor, fibrosarcoma of bone, chordoma, periosteal sarcoma, soft-tissue sarcomas, angiosarcoma (hemangiosarcoma), fibrosarcoma, Kaposi's sarcoma, leiomyosarcoma, liposarcoma, lymphangiosarcoma, neurilemmoma, rhabdomyosarcoma, synovial sarcoma; brain tumors including, but not limited to, glioma, astrocytoma, brain stem glioma, ependymoma, oligodendroglioma, nonglial tumor, acoustic neurinoma, craniopharyngioma, medulloblastoma, meningioma, pineocytoma, pineoblastoma, primary brain lymphoma; breast cancer including, but not limited to, adenocarcinoma, lobular (small cell) carcinoma, intraductal carcinoma, medullary breast cancer, mucinous breast cancer, tubular breast cancer, papillary breast cancer, Paget's disease, and inflammatory breast cancer; adrenal cancer, including, but not limited to, pheochromocytom and adrenocortical carcinoma; thyroid cancer such as but not limited to papillary or follicular thyroid cancer, medullary thyroid cancer and anaplastic thyroid cancer; pancreatic cancer, including, but not limited to, insulinoma, gastrinoma, glucagonoma, vipoma, somatostatin-secreting tumor, and carcinoid or islet cell tumor; pituitary cancers including, but not limited to, Cushing's disease, prolactin-secreting tumor, acromegaly, and diabetes insipius; eye cancers including, but not limited to, ocular melanoma such as iris melanoma, choroidal melanoma, and ciliary body melanoma, and retinoblastoma; vaginal cancers, including, but not limited to, squamous cell carcinoma, adenocarcinoma, and melanoma; vulvar cancer, including, but not limited to, squamous cell carcinoma, melanoma, adenocarcinoma, basal cell carcinoma, sarcoma, and Paget's disease; cervical cancers including, but not limited to, squamous cell carcinoma, and adenocarcinoma; uterine cancers including, but not limited to, endometrial carcinoma and uterine sarcoma; ovarian cancers including, but not limited to, ovarian epithelial carcinoma, borderline tumor, germ cell tumor, and stromal tumor; esophageal cancers including, but not limited to, squamous cancer, adenocarcinoma, adenoid cyctic carcinoma, mucoepidermoid carcinoma, adenosquamous carcinoma, sarcoma, melanoma, plasmacytoma, verrucous carcinoma, and oat cell (small cell) carcinoma; stomach cancers including, but not limited to, adenocarcinoma, fungating (polypoid), ulcerating, superficial spreading, diffusely spreading, malignant lymphoma, liposarcoma, fibrosarcoma, and carcinosarcoma; colon cancers; rectal cancers; liver cancers including, but not limited to, hepatocellular carcinoma and hepatoblastoma, gallbladder cancers including, but not limited to, adenocarcinoma; cholangiocarcinomas including, but not limited to, papillary, nodular, and diffuse; lung cancers including, but not limited to, non-small cell lung cancer, squamous cell carcinoma (epidermoid carcinoma), adenocarcinoma, large-cell carcinoma and small-cell lung cancer; testicular cancers including, but not limited to, germinal tumor, seminoma, anaplastic, classic (typical), spermatocytic, nonseminoma, embryonal carcinoma, teratoma carcinoma, choriocarcinoma (yolk-sac tumor), prostate cancers including, but not limited to, adenocarcinoma, leiomyosarcoma, and rhabdomyosarcoma; penal cancers; oral cancers including, but not limited to, squamous cell carcinoma; basal cancers; salivary gland cancers including, but not limited to, adenocarcinoma, mucoepidermoid carcinoma, and adenoidcystic carcinoma; pharynx cancers including, but not limited to, squamous cell cancer, and verrucous; skin cancers including, but not limited to, basal cell carcinoma, squamous cell carcinoma and melanoma, superficial spreading melanoma, nodular melanoma, lentigo malignant melanoma, acral lentiginous melanoma; kidney cancers including, but not limited to, renal cell cancer, adenocarcinoma, hypernephroma, fibrosarcoma, transitional cell cancer (renal pelvis and/or uterer); Wilms' tumor; bladder cancers including, but not limited to, transitional cell carcinoma, squamous cell cancer, adenocarcinoma, and carcinosarcoma. For a review of such disorders, see Fishman et al., 1985, Medicine, 2d Ed., J.B. Lippincott Co., Philadelphia and Murphy et al., 1997, Informed Decisions: The Complete Book of Cancer Diagnosis, Treatment, and Recovery, Viking Penguin, Penguin Books U.S.A., Inc., United States of America).

2. Immune System Disorders

Immune system disorders can be treated in accordance with the disclosed compositions and methods. Non-limiting examples of immune system disorders include 22q11.2 deletion syndrome, Achondroplasia and severe combined immunodeficiency, Adenosine Deaminase 2 deficiency, Adenosine deaminase deficiency, Adult-onset immunodeficiency with anti-interferon-gamma autoantibodies, Agammaglobulinemia, non-Bruton type, Aicardi-Goutieres syndrome, Aicardi-Goutieres syndrome type 5, Allergic bronchopulmonary aspergillosis, Alopecia, Alopecia totalis, Alopecia universalis, Amyloidosis AA, Amyloidosis familial visceral, Ataxia telangiectasia, Autoimmune lymphoproliferative syndrome, Autoimmune lymphoproliferative syndrome due to CTLA4 haploinsuffiency, Autoimmune polyglandular syndrome type 1, Autosomal dominant hyper IgE syndrome, Autosomal recessive early-onset inflammatory bowel disease, Autosomal recessive hyper IgE syndrome, Bare lymphocyte syndrome 2, Barth syndrome, Blau syndrome, Bloom syndrome, Bronchiolitis obliterans, C1q deficiency, Candidiasis familial chronic mucocutaneous, autosomal recessive, Cartilage-hair hypoplasia, CHARGE syndrome, Chediak-Higashi syndrome, Cherubism, Chronic atypical neutrophilic dermatosis with lipodystrophy and elevated temperature, Chronic graft versus host disease, Chronic granulomatous disease, Chronic Infantile Neurological Cutaneous Articular syndrome, Chronic mucocutaneous candidiasis (CMC), Cohen syndrome, Combined immunodeficiency with skin granulomas, Common variable immunodeficiency, Complement component 2 deficiency, Complement component 8 deficiency type 1, Complement component 8 deficiency type 2, Congenital pulmonary alveolar proteinosis, Cryoglobulinemia, Cutaneous mastocytoma, Cyclic neutropenia, Deficiency of interleukin-1 receptor antagonist, Dendritic cell, monocyte, B lymphocyte, and natural killer lymphocyte deficiency, Dyskeratosis congenital, Dyskeratosis congenita autosomal dominant, Dyskeratosis congenita autosomal recessive, Dyskeratosis congenita X-linked, Epidermodysplasia verruciformis, Familial amyloidosis, Finnish type, Familial cold autoinflammatory syndrome, Familial Mediterranean fever, Familial mixed cryoglobulinemia, Felty's syndrome, Glycogen storage disease type 1B, Griscelli syndrome type 2, Hashimoto encephalopathy, Hashimoto's syndrome, Hemophagocytic lymphohistiocytosis, Hennekam syndrome, Hepatic venoocclusive disease with immunodeficiency, Hereditary folate malabsorption, Hermansky Pudlak syndrome 2, Herpes simplex encephalitis, Hoyeraal Hreidarsson syndrome, Hyper IgE syndrome, Hyper-IgD syndrome, ICF syndrome, Idiopathic acute eosinophilic pneumonia, Idiopathic CD4 positive T-lymphocytopenia, IL12RB1 deficiency, Immune defect due to absence of thymus, Immune dysfunction with T-cell inactivation due to calcium entry defect 1, Immune dysfunction with T-cell inactivation due to calcium entry defect 2, Immunodeficiency with hyper IgM type 1, Immunodeficiency with hyper IgM type 2, Immunodeficiency with hyper IgM type 3, Immunodeficiency with hyper IgM type 4, Immunodeficiency with hyper IgM type 5, Immunodeficiency with thymoma, Immunodeficiency without anhidrotic ectodermal dysplasia, Immunodysregulation, polyendocrinopathy and enteropathy X-linked, Immunoglobulin A deficiency 2, Intestinal atresia multiple, IRAK-4 deficiency, Isolated growth hormone deficiency type 3, Kawasaki disease, Large granular lymphocyte leukemia, Leukocyte adhesion deficiency type 1, LRBA deficiency, Lupus, Lymphocytic hypophysitis, Majeed syndrome, Melkersson-Rosenthal syndrome, MHC class 1 deficiency, Muckle-Wells syndrome, Multifocal fibrosclerosis, Multiple sclerosis, MYD88 deficiency, Neonatal systemic lupus erythematosus, Netherton syndrome, Neutrophil-specific granule deficiency, Nijmegen breakage syndrome, Omenn syndrome, Osteopetrosis autosomal recessive 7, Palindromic rheumatism, Papillon Lefevre syndrome, Partial androgen insensitivity syndrome, PASLI disease, Pearson syndrome, Pediatric multiple sclerosis, Periodic fever, aphthous stomatitis, pharyngitis and adenitis, PGM3-CDG, Poikiloderma with neutropenia, Pruritic urticarial papules plaques of pregnancy, Purine nucleoside phosphorylase deficiency, Pyogenic arthritis, pyoderma gangrenosum and acne, Relapsing polychondritis, Reticular dysgenesis, Sarcoidosis, Say Barber Miller syndrome, Schimke immunoosseous dysplasia, Schnitzler syndrome, Selective IgA deficiency, Selective IgM deficiency, Severe combined immunodeficiency, Severe combined immunodeficiency due to complete RAG1/2 deficiency, Severe combined immunodeficiency with sensitivity to ionizing radiation, Severe combined immunodeficiency, Severe congenital neutropenia autosomal recessive 3, Severe congenital neutropenia X-linked, Shwachman-Diamond syndrome, Singleton-Merten syndrome, SLC35C1-CDG (CDG-IIc), Specific antibody deficiency, Spondyloenchondrodysplasia, Stevens-Johnson syndrome, T-cell immunodeficiency, congenital alopecia and nail dystrophy, TARP syndrome, Trichohepatoenteric syndrome, Tumor necrosis factor receptor-associated periodic syndrome, Twin to twin transfusion syndrome, Vici syndrome, WHIM syndrome, Wiskott Aldrich syndrome, Woods Black Norbury syndrome, X-linked agammaglobulinemia, X-linked lymphoproliferative syndrome, X-linked lymphoproliferative syndrome 1, X-linked lymphoproliferative syndrome 2, X-linked magnesium deficiency with Epstein-Barr virus infection and neoplasia, X-linked severe combined immunodeficiency, and ZAP-70 deficiency.

The disclosed compositions and methods can also be used to treat autoimmune diseases or disorders. Exemplary autoimmune diseases or disorders, which are not mutually exclusive with the immune system disorders described above, include Achalasia, Addison's disease, Adult Still's disease, Agammaglobulinemia, Alopecia areata, Amyloidosis, Ankylosing spondylitis, Anti-GBM/Anti-TBM nephritis, Antiphospholipid syndrome, Autoimmune angioedema, Autoimmune dysautonomia, Autoimmune encephalomyelitis, Autoimmune hepatitis, Autoimmune inner ear disease (AIED), Autoimmune myocarditis, Autoimmune oophoritis, Autoimmune orchitis, Autoimmune pancreatitis, Autoimmune retinopathy, Autoimmune urticarial, Axonal & neuronal neuropathy (AMAN), Baló disease, Behcet's disease, Benign mucosal pemphigoid, Bullous pemphigoid, Castleman disease (CD), Celiac disease, Chagas disease, Chronic inflammatory demyelinating polyneuropathy (CIDP), Chronic recurrent multifocal osteomyelitis (CRMO), Churg-Strauss Syndrome (CSS) or Eosinophilic Granulomatosis (EGPA), Cicatricial pemphigoid, Cogan's syndrome, Cold agglutinin disease, Congenital heart block, Coxsackie myocarditis, CREST syndrome, Crohn's disease, Dermatitis herpetiformis, Dermatomyositis, Devic's disease (neuromyelitis optica), Discoid lupus, Dressler's syndrome, Endometriosis, Eosinophilic esophagitis (EoE), Eosinophilic fasciitis, Erythema nodosum, Essential mixed cryoglobulinemia, Evans syndrome, Fibromyalgia, Fibrosing alveolitis, Giant cell arteritis (temporal arteritis), Giant cell myocarditis, Glomerulonephritis, Goodpasture's syndrome, Granulomatosis with Polyangiitis, Graves' disease, Guillain-Barre syndrome, Hashimoto's thyroiditis, Hemolytic anemia, Henoch-Schonlein purpura (HSP), Herpes gestationis or pemphigoid gestationis (PG), Hidradenitis Suppurativa (HS) (Acne Inversa), Hypogammalglobulinemia, IgA Nephropathy, IgG4-related sclerosing disease, Immune thrombocytopenic purpura (ITP), Inclusion body myositis (IBM), Interstitial cystitis (IC), Juvenile arthritis, Juvenile diabetes (Type 1 diabetes), Juvenile myositis (JM), Kawasaki disease, Lambert-Eaton syndrome, Leukocytoclastic vasculitis, Lichen planus, Lichen sclerosus, Ligneous conjunctivitis, Linear IgA disease (LAD), Lupus, Lyme disease chronic, Meniere's disease, Microscopic polyangiitis (MPA), Mixed connective tissue disease (MCTD), Mooren's ulcer, Mucha-Habermann disease, Multifocal Motor Neuropathy (MMN) or MMNCB, Multiple sclerosis, Myasthenia gravis, Myositis, Narcolepsy, Neonatal Lupus, Neuromyelitis optica, Neutropenia, Ocular cicatricial pemphigoid, Optic neuritis, Palindromic rheumatism (PR), PANDAS, Paraneoplastic cerebellar degeneration (PCD), Paroxysmal nocturnal hemoglobinuria (PNH), Parry Romberg syndrome, Pars planitis (peripheral uveitis), Parsonnage-Turner syndrome, Pemphigus, Peripheral neuropathy, Perivenous encephalomyelitis, Pernicious anemia (PA), POEMS syndrome, Polyarteritis nodosa, Polyglandular syndromes type I, II, III, Polymyalgia rheumatic, Polymyositis, Postmyocardial infarction syndrome, Postpericardiotomy syndrome, Primary biliary cirrhosis, Primary sclerosing cholangitis, Progesterone dermatitis, Psoriasis, Psoriatic arthritis, Pure red cell aplasia (PRCA), Pyoderma gangrenosum, Raynaud's phenomenon, Reactive Arthritis, Reflex sympathetic dystrophy, Relapsing polychondritis, Restless legs syndrome (RLS), Retroperitoneal fibrosis, Rheumatic fever, Rheumatoid arthritis, Sarcoidosis, Schmidt syndrome, Scleritis, Scleroderma, Sjögren's syndrome, Sperm & testicular autoimmunity, Stiff person syndrome (SPS), Subacute bacterial endocarditis (SBE), Susac's syndrome, Sympathetic ophthalmia (SO), Takayasu's arteritis, Temporal arteritis/Giant cell arteritis, Thrombocytopenic purpura (TTP), Tolosa-Hunt syndrome (THS), Transverse myelitis, Type 1 diabetes, Ulcerative colitis (UC), Undifferentiated connective tissue disease (UCTD), Uveitis, Vasculitis, Vitiligo, Vogt-Koyanagi-Harada Disease, and Wegener's granulomatosis (or Granulomatosis with Polyangiitis (GPA)).

B. Effective Amounts

The effective amount or therapeutically effective amount of a disclosed pharmaceutical composition can be a dosage sufficient to treat, inhibit, or alleviate one or more symptoms of a disease or disorder, or to otherwise provide a desired pharmacologic and/or physiologic effect, for example, reducing, inhibiting, or reversing one or more of the underlying pathophysiological mechanisms underlying a disease or disorder such as cancer.

In some forms, administration of the pharmaceutical compositions elicits an anti-cancer response, the amount administered can be expressed as the amount effective to achieve a desired anti-cancer effect in the recipient. For example, in some forms, the amount of the pharmaceutical compositions is effective to inhibit the viability or proliferation of cancer cells in the recipient. In some forms, the amount of pharmaceutical compositions is effective to reduce the tumor burden in the recipient, or reduce the total number of cancer cells, and combinations thereof. In other forms, the amount of the pharmaceutical compositions is effective to reduce one or more symptoms or signs of cancer in a cancer patient. Signs of cancer can include cancer markers, such as PSMA levels in the blood of a patient.

The effective amount of the pharmaceutical compositions required will vary from subject to subject, depending on the species, age, weight and general condition of the subject, the severity of the disorder being treated, and its mode of administration. Thus, it is not possible to specify an exact amount for every pharmaceutical composition. However, an appropriate amount can be determined by one of ordinary skill in the art using only routine experimentation given the teachings herein. For example, effective dosages and schedules for administering the pharmaceutical compositions can be determined empirically, and making such determinations is within the skill in the art. In some forms, the dosage ranges for the administration of the compositions are those large enough to effect reduction in cancer cell proliferation or viability, or to reduce tumor burden for example.

The dosage should not be so large as to cause adverse side effects, such as unwanted cross-reactions, anaphylactic reactions, and the like. Generally, the dosage will vary with the age, condition, and sex of the patient, route of administration, whether other drugs are included in the regimen, and the type, stage, and location of the disease to be treated. The dosage can be adjusted by the individual physician in the event of any counter-indications. It will also be appreciated that the effective dosage of the composition used for treatment can increase or decrease over the course of a particular treatment. Changes in dosage can result and become apparent from the results of diagnostic assays.

Dosage can vary, and can be administered in one or more dose administrations daily, for one or several days. Guidance can be found in the literature for appropriate dosages for given classes of pharmaceutical products. Optimal dosing schedules can be calculated from measurements of drug accumulation in the body of the subject or patient. Persons of ordinary skill can easily determine optimum dosages, dosing methodologies and repetition rates. Optimum dosages can vary depending on the relative potency of individual pharmaceutical compositions, and can generally be estimated based on EC₅₀s found to be effective in in vitro and in vivo animal models.

It can generally be stated that a pharmaceutical composition containing the CAR T cells described herein can be administered at a dosage of 10⁴ to 10⁹ cells/kg body weight, preferably 10⁵ to 10⁶ cells/kg body weight, including all integer values within those ranges. In some forms, patients can be treated by infusing a disclosed pharmaceutical composition containing CAR expressing cells (e.g., T cells) in the range of about 10⁴ to 10¹² or more cells per square meter of body surface (cells/m). The infusion can be repeated as often and as many times as the patient can tolerate until the desired response is achieved. CAR T cell compositions can also be administered once or multiple times at these dosages. The cells can be administered by using infusion techniques that are commonly known in immunotherapy (see, e.g., Rosenberg et al., New Eng. J. of Med. 319:1676, 1988). The optimal dosage and treatment regime for a particular patient can readily be determined by one skilled in the art of medicine by monitoring the patient for signs of disease and adjusting the treatment accordingly. In some forms, the unit dosage is in a unit dosage form for intravenous injection. In some forms, the unit dosage is in a unit dosage form for oral administration. In some forms, the unit dosage is in a unit dosage form for inhalation. In some forms, the unit dosage is in a unit dosage form for intratumoral injection.

Treatment can be continued for an amount of time sufficient to achieve one or more desired therapeutic goals, for example, a reduction of the amount of cancer cells relative to the start of treatment, or complete absence of cancer cells in the recipient. Treatment can be continued for a desired period of time, and the progression of treatment can be monitored using any means known for monitoring the progression of anti-cancer treatment in a patient. In some forms, administration is carried out every day of treatment, or every week, or every fraction of a week. In some forms, treatment regimens are carried out over the course of up to two, three, four or five days, weeks, or months, or for up to 6 months, or for more than 6 months, for example, up to one year, two years, three years, or up to five years.

The efficacy of administration of a particular dose of the pharmaceutical compositions according to the methods described herein can be determined by evaluating the particular aspects of the medical history, signs, symptoms, and objective laboratory tests that are known to be useful in evaluating the status of a subject in need for the treatment of cancer or other diseases and/or conditions. These signs, symptoms, and objective laboratory tests will vary, depending upon the particular disease or condition being treated or prevented, as will be known to any clinician who treats such patients or a researcher conducting experimentation in this field. For example, if, based on a comparison with an appropriate control group and/or knowledge of the normal progression of the disease in the general population or the particular individual: (1) a subject's physical condition is shown to be improved (e.g., a tumor has partially or fully regressed), (2) the progression of the disease or condition is shown to be stabilized, or slowed, or reversed, or (3) the need for other medications for treating the disease or condition is lessened or obviated, then a particular treatment regimen will be considered efficacious. In some forms, efficacy is assessed as a measure of the reduction in tumor volume and/or tumor mass at a specific time point (e.g., 1-5 days, weeks or months) following treatment.

C. Modes of Administration

Any of the disclosed genetically modified cells (e.g., CAR T cells) can be used therapeutically in combination with a pharmaceutically acceptable carrier. The compositions described herein can be conveniently formulated into pharmaceutical compositions composed of one or more of the compounds in association with a pharmaceutically acceptable carrier. See, e.g., Remington's Pharmaceutical Sciences, latest edition, by E.W. Martin Mack Pub. Co., Easton, Pa., which discloses typical carriers and conventional methods of preparing pharmaceutical compositions that can be used in conjunction with the preparation of formulations of the therapeutics described herein and which is incorporated by reference herein. These most typically would be standard carriers for administration of compositions to humans. In one aspect, for humans and non-humans, these include solutions such as sterile water, saline, and buffered solutions at physiological pH. Other therapeutics can be administered according to standard procedures used by those skilled in the art.

The pharmaceutical compositions described herein can include, but are not limited to, carriers, thickeners, diluents, buffers, preservatives, surface active agents and the like in addition to the therapeutic(s) of choice.

Pharmaceutical compositions containing one or more therapeutics can be administered to the subject in a number of ways depending on whether local or systemic treatment is desired, and on the area to be treated. Thus, for example, a pharmaceutical composition can be administered as an ophthalmic solution and/or ointment to the surface of the eye. Moreover, a pharmaceutical composition can be administered to a subject vaginally, rectally, intranasally, orally, by inhalation, or parenterally, for example, by intradermal, subcutaneous, intramuscular, intraperitoneal, intrarectal, intraarterial, intralymphatic, intravenous, intrathecal and intratracheal routes. The compositions can be administered directly into a tumor or tissue, e.g., stereotactically.

Parenteral administration, if used, is generally characterized by injection. Injectables can be prepared in conventional forms, either as liquid solutions or suspensions, solid forms suitable for solution or suspension in liquid prior to injection, or as emulsions. A more recently revised approach for parenteral administration involves use of a slow release or sustained release system such that a constant dosage is maintained. See, e.g., U.S. Pat. No. 3,610,795, which is incorporated by reference herein. Suitable parenteral administration routes include intravascular administration (e.g., intravenous bolus injection, intravenous infusion, intra-arterial bolus injection, intra-arterial infusion and catheter instillation into the vasculature); peri- and intra-tissue injection (e.g., intraocular injection, intra-retinal injection, or sub-retinal injection); subcutaneous injection or deposition including subcutaneous infusion (such as by osmotic pumps); direct application by a catheter or other placement device (e.g., an implant comprising a porous, non-porous, or gelatinous material).

Preparations for parenteral administration include sterile aqueous or non-aqueous solutions, suspensions, and emulsions which can also contain buffers, diluents and other suitable additives. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives can also be present such as, for example, antimicrobials, anti-oxidants, chelating agents, and inert gases and the like.

Administration of the pharmaceutical compositions containing one or more genetically modified cells (e.g., CAR T cells) can be localized (i.e., to a particular region, physiological system, tissue, organ, or cell type) or systemic.

It is to be understood that the disclosed method and compositions are not limited to specific synthetic methods, specific analytical techniques, or to particular reagents unless otherwise specified, and, as such, can vary. It is also to be understood that the terminology used herein is for the purpose of describing particular embodiments only and is not intended to be limiting.

D. Combination Therapy

Any of the disclosed pharmaceutical compositions (e.g., containing a population of CAR cells) can be used alone, or in combination with other therapeutic agents or treatment modalities, for example, chemotherapy or stem-cell transplantation. As used herein, “combination” or “combined” refer to either concomitant, simultaneous, or sequential administration of the therapeutics.

In some forms, the pharmaceutical compositions and other therapeutic agents are administered separately through the same route of administration. In other forms, the pharmaceutical compositions and other therapeutic agents are administered separately through different routes of administration. The combinations can be administered either concomitantly (e.g., as an admixture), separately but simultaneously (e.g., via separate intravenous lines into the same subject; one agent is given orally while the other agent is given by infusion or injection, etc.), or sequentially (e.g., one agent is given first followed by the second).

Examples of preferred additional therapeutic agents include other conventional therapies known in the art for treating the desired disease, disorder or condition. In some forms, the therapeutic agent is one or more other targeted therapies (e.g., a targeted cancer therapy) and/or immune-checkpoint blockage agents (e.g., anti-CTLA-4, anti-PD1, and/or anti-PDL1 agents such as antibodies). In the context of cancer, targeted therapies are therapeutic agents that block the growth and spread of cancer by interfering with specific molecules (“molecular targets”) that are involved in the growth, progression, and spread of cancer. Many different targeted therapies have been approved for use in cancer treatment. These therapies include hormone therapies, signal transduction inhibitors, gene expression modulators, apoptosis inducers, angiogenesis inhibitors, immunotherapies, and toxin delivery molecules. Numerous antineoplastic drugs can be used in combination with the disclosed pharmaceutical compositions. In some forms, the additional therapeutic agent is a chemotherapeutic or antineoplastic drug. The majority of chemotherapeutic drugs can be divided into alkylating agents, antimetabolites, anthracyclines, plant alkaloids, topoisomerase inhibitors, monoclonal antibodies, and other antitumour agents.

The compositions and methods described herein may be used as a first therapy, second therapy, third therapy, or combination therapy with other types of therapies known in the art, such as chemotherapy, surgery, radiation, gene therapy, immunotherapy, bone marrow transplantation, stem cell transplantation, targeted therapy, cryotherapy, ultrasound therapy, photodynamic therapy, radio-frequency ablation or the like, in an adjuvant setting or a neoadjuvant setting.

The disclosed pharmaceutical compositions and/or other therapeutic agents, procedures or modalities can be administered during periods of active disease, or during a period of remission or less active disease. The pharmaceutical compositions can be administered before the additional treatment, concurrently with the treatment, post-treatment, or during remission of the disease or disorder. When administered in combination, the disclosed pharmaceutical compositions and the additional therapeutic agents (e.g., second or third agent), or all, can be administered in an amount or dose that is higher, lower or the same than the amount or dosage of each agent used individually, e.g., as a monotherapy. In certain forms, the administered amount or dosage of the disclosed pharmaceutical composition, the additional therapeutic agent (e.g., second or third agent), or all, is lower (e.g., at least 20%, at least 30%, at least 40%, or at least 50%) than the amount or dosage of each agent used individually, e.g., as a monotherapy (e.g., required to achieve the same therapeutic effect).

The disclosed compositions and methods can be further understood through the following numbered paragraphs.

1. A method of modifying the genome of a cell comprising introducing to the cell an RNA-guided endonuclease, and

one or more AAV vectors at least one of which comprises a sequence that encodes one or more crRNAs, wherein the one or more crRNAs collectively direct the RNA-guided endonuclease to one or more target genes;

and optionally, wherein at least one of the AAV vectors comprises or further comprises one or more HDR templates.

2. The method of paragraph 1, wherein two or more of the crRNAs are encoded by a crRNA array.
3. The method of paragraph 2, wherein each of the two or more crRNAs encoded by the crRNA array direct the RNA-guided endonuclease to a different target gene.
4. The method of any one of paragraphs 1-3, wherein two AAV vectors are introduced to the cell.
5. The method of any one of paragraphs 1-4, wherein at least one of the HDR templates comprises:

(a) a sequence that encodes a reporter gene, a chimeric antigen receptor (CAR), or combinations thereof; and

(b) one or more sequences collectively homologous to one or more target sites.

6. The method of paragraph 5, wherein the sequence in (a) further comprises a promoter and/or polyadenylation signal operationally linked to the reporter gene and the CAR.
7. The method of paragraph 5 or 6, wherein the RNA-guided endonuclease induces disruption of the target genes and/or the one or more HDR templates mediate targeted integration of the reporter gene, the CAR, or a combination thereof, at the target sites.
8. The method of paragraph 7, wherein the target site is within the locus of the disrupted gene.
9. The method of paragraph 7, wherein the target site is at a locus different from the disrupted gene.
10. The method of paragraph 7, wherein the target gene or target site comprises PDCD1, or TRAC genes.
11. The method of paragraph 10, wherein

(a) the PDCD1 or TRAC gene is disrupted;

(b) the PDCD1 and TRAC genes are disrupted;

(c) the reporter gene, CAR, or combination thereof, is integrated in the PDCD1 or TRAC gene;

(d) the reporter genes, CARs, or combination thereof are integrated in both the PDCD1 and TRAC genes;

(e) the PDCD1 gene is disrupted and the reporter gene, CAR, or combination thereof, is integrated in the TRAC gene; or

(f) the TRAC gene is disrupted and the reporter gene, CAR, or combination thereof, is integrated in the PDCD1 gene.

12. The method of any one of paragraphs 5-11, wherein the CAR targets one or more antigens specific for cancer, an inflammatory disease, a neuronal disorder, HIV/AIDS, diabetes, a cardiovascular disease, an infectious disease, an autoimmune disease, or combinations thereof.
13. The method of paragraph 12, wherein the CAR is bispecific or multivalent. 14. The method of paragraph 12 or 13, wherein the CAR targets one or more antigens selected from the group comprising AFP, AKAP-4, ALK, Androgen receptor, B7H3, BCMA, Bcr-Abl, BORIS, Carbonic, CD123, CD138, CD174, CD19, CD20, CD22, CD30, CD33, CD38, CD80, CD86, CEA, CEACAMS, CEACAM6, Cyclin, CYP1B1, EBV, EGFR, EGFR806, EGFRvIII, EpCAM, EphA2, ERG, ETV6-AML, FAP, Fos-related antigenl, Fucosyl, fusion, GD2, GD3, GloboH, GM3, gp100, GPC3, HER-2/neu, HER2, HMWMAA, HPV E6/E7, hTERT, Idiotype, IL12, IL13RA2, IM19, IX, LCK, Legumain, IgK, LMP2, MAD-CT-1, MAD-CT-2, MAGE, MelanA/MART1, Mesothelin, MET, ML-IAP, MUC1, Mutant p53, MYCN, NA17, NKG2D-L, NY-BR-1, NY-ESO-1, NY-ESO-1, OY-TES1, p53, Page4, PAP, PAX3, PAXS, PD-L1, PDGFR-β, PLAC1, Polysialic acid, Proteinase3 (PR1), PSA, PSCA, PSMA, Ras mutant, RGSS, RhoC, ROR1, SART3, sLe(a), Sperm protein 17, SSX2, STn, Survivin, Tie2, Tn, TRP-2, Tyrosinase, VEGFR2, WT1, and XAGE.
15. The method of paragraph 14, wherein the CAR is anti-CD19 or anti-CD22.
16. The method of paragraph 15, wherein the CAR is CD19BBz or CD22BBz.
17. The method of any one of paragraphs 1-16, wherein the RNA-guided endonuclease is provided as an mRNA that encodes the RNA-guided endonuclease, a viral vector that encodes the RNA-guided endonuclease, or an RNA-guided endonuclease protein or a complex of the RNA-guided endonuclease protein and RNA.
18. The method of paragraph 17, wherein the mRNA comprises N6-methyladenosine (m6A), 5-methylcytosine (m5C), pseudouridine (w), N1-methylpseudouridine (me liv), 5-methoxyuridine (5moU), a 5′ cap, a poly(A) tail, one or more nuclear localization signals, or combinations thereof.
19. The method of paragraph 17 or 18, wherein the mRNA is codon optimized for expression in a eukaryotic cell.
20. The method of paragraph 19, wherein the mRNA is introduced to the cell by electroporation, transfection, or nanoparticle mediated delivery.
21. The method of any one of paragraphs 17-20, wherein the RNA-guided endonuclease is Cpf1 or an active variant, derivative, or fragment thereof.
22. The method of paragraph 21, wherein the Cpf1 is derived from Francisella novicida U112 (FnCpf1), Acidaminococcus sp. BV3L6 (AsCpf1), Lachnospiraceae bacterium ND2006 (LbCpf1), Lachnospiraceae bacterium MA2020 (Lb2Cpf1), Lachnospiraceae bacterium MC2017 (Lb3Cpf1), Moraxella bovoculi 237 (MbCpf1), Butyrivibrio proteoclasticus (BpCpf1), Parcubacteria bacterium GWC2011_GWC2_44_17 (PbCpf1); Peregrinibacteria bacterium GW2011_GWA_33_10 (PeCpf1), Leptospira inadai (LiCpf1), Smithella sp. SC_K08D17 (SsCpf1), Porphyromonas crevioricanis (PcCpf1), Porphyromonas macacae (PmCpf1), Candidatus Methanoplasma termitum (CMtCpf1), Eubacterium eligens (EeCpf1), Moraxella bovoculi 237 (MbCpf1), or Prevotella disiens (PdCpf1).
23. The method of paragraph 22, wherein the Cpf1 is LbCpf1, or an active variant, derivative, or fragment thereof.
24. The method of any one of paragraphs 1-23, wherein at least one of the AAV vectors is AAV6 or AAV9.
25. The method of any one of paragraphs 1-24, wherein the introduction is performed ex vivo.
26. The method of paragraph 25, wherein the RNA-guided endonuclease and the one or more AAV vectors are introduced to the cell at the same or different times.
27. The method of any one of paragraphs 1-26, wherein the cell is a T cell, hematopoietic stem cell (HSC), macrophage, natural killer cell (NK), or dendritic cell (DC).
28. The method of paragraph 27, wherein the T cell is a CD8+ T cell selected from the group consisting of effector T cells, memory T cells, central memory T cells, and effector memory T cells.
29. The method of paragraph 27, wherein the T cell is a CD4+ T cell selected from the group consisting of Th1 cells, Th2 cells, Th17 cells, and Treg cells.
30. An isolated cell modified according to the method of any one of paragraphs 1-29.
31. The isolated cell of paragraph 30, wherein the cell is bispecific or multispecific.
32. A population of cells derived by expanding the cell of paragraph 30 or 31.
33. A pharmaceutical composition comprising the population of cells of paragraph 32 and a pharmaceutically acceptable buffer, carrier, diluent or excipient.
34. A method of treating a subject having a disease, disorder, or condition comprising administering to the subject an effective amount of the pharmaceutical composition of paragraph 33.
35. A method of treating a subject having a disease, disorder, or condition associated with an elevated expression or specific expression of an antigen, the method comprising administering to the subject an effective amount of a T cell modified according to the method of any one of paragraphs 1-26, wherein the T cell comprises a CAR that targets the antigen.
36. A method of treating a subject having a disease, disorder, or condition comprising administering to the subject an effective amount of a pharmaceutical composition comprising a genetically modified cell, wherein the cell is genetically modified by a method comprising introducing to the cell:

(a) an RNA-guided endonuclease; and

(b) one or more AAV vectors at least one of which comprises
(i) a sequence that encodes one or more crRNAs, wherein the one or more crRNAs collectively direct the RNA-guided endonuclease to one or more target genes; and
(ii) one or more HDR templates at least one of which comprises a sequence that encodes one or more chimeric antigen receptors (CAR); and
(iii) one or more sequences at least one of which is homologous to a target site.
   37. The method of paragraph 36, wherein the RNA-guided endonuclease induces disruption of the one or more target genes and wherein the one or more CARs are integrated at the target site.
   38. The method of paragraph 37, wherein the target site is within the locus of one of the disrupted genes.
   39. The method of paragraph 37, wherein the target site is at a locus different from the disrupted genes.
   40. The method of any one of paragraphs 36-39, wherein the target gene or target site comprises PDCD1 or TRAC genes.
   41. The method of paragraph 40, wherein

(a) the PDCD1 or TRAC gene is disrupted;

(b) the PDCD1 and TRAC genes are disrupted;

(c) the one or more CARs are integrated within the PDCD1 or TRAC gene;

(d) the one or more CARs are integrated within both the PDCD1 and TRAC gene;

(e) the PDCD1 gene is disrupted and the one or more CARs are integrated in the TRAC gene; or

(f) the TRAC gene is disrupted and the one or more CARs are integrated in the PDCD1 gene.

42. The method of any one of paragraphs 36-41, wherein at least one of the CARs targets one or more antigens specific for or associated with the disease, disorder, or condition.
43. The method of paragraph 42, wherein the disease, disorder, or condition is a cancer, an inflammatory disease, a neuronal disorder, HIV/AIDS, diabetes, a cardiovascular disease, an infectious disease, or an autoimmune disease.
44. The method of paragraph 43, wherein the cancer is a leukemia or lymphoma selected from the group comprising chronic lymphocytic leukemia (CLL), acute lymphocytic leukemia (ALL), acute myeloid leukemia (AML), chronic myelogenous leukemia (CML), mantle cell lymphoma, non-Hodgkin's lymphoma, and Hodgkin's lymphoma.

45. The method of any one of paragraphs 42-44, wherein the at least one of the CARs is bispecific or multivalent.

46. The method of any one of paragraphs 43-45, wherein the at least one of the CARs targets one or more antigens selected from the group comprising AFP, AKAP-4, ALK, Androgen receptor, B7H3, BCMA, Bcr-Abl, BORIS, Carbonic, CD123, CD138, CD174, CD19, CD20, CD22, CD30, CD33, CD38, CD80, CD86, CEA, CEACAMS, CEACAM6, Cyclin, CYP1B1, EBV, EGFR, EGFR806, EGFRvIII, EpCAM, EphA2, ERG, ETV6-AML, FAP, Fos-related antigenl, Fucosyl, fusion, GD2, GD3, GloboH, GM3, gp100, GPC3, HER-2/neu, HER2, HMWMAA, HPV E6/E7, hTERT, Idiotype, IL12, IL13RA2, IM19, IX, LCK, Legumain, IgK, LMP2, MAD-CT-1, MAD-CT-2, MAGE, MelanA/MART1, Mesothelin, MET, ML-IAP, MUC1, Mutant p53, MYCN, NA17, NKG2D-L, NY-BR-1, NY-ESO-1, NY-ESO-1, OY-TES1, p53, Page4, PAP, PAX3, PAXS, PD-L1, PDGFR-β, PLAC1, Polysialic acid, Proteinase3 (PR1), PSA, PSCA, PSMA, Ras mutant, RGSS, RhoC, ROR1, SART3, sLe(a), Sperm protein 17, SSX2, STn, Survivin, Tie2, Tn, TRP-2, Tyrosinase, VEGFR2, WT1, and XAGE.
47. The method of paragraph 46, wherein the at least one of the CARs is anti-CD19 or anti-CD22.
48. The method of paragraph 47, wherein the at least one of the CARs is CD19BBz or CD22BBz.
49. The method of any one of paragraphs 36-48, wherein the RNA-guided endonuclease is provided as an mRNA that encodes the RNA-guided endonuclease, a viral vector that encodes the RNA-guided endonuclease, or an RNA-guided endonuclease protein or a complex of the RNA-guided endonuclease protein and RNA.
50. The method of paragraph 49, wherein the mRNA is introduced to the cell by electroporation, transfection, or nanoparticle mediated delivery.
51. The method of 49 or 50, wherein the RNA-guided endonuclease is LbCpf1, or an active variant, derivative, or fragment thereof.
52. The method of any one of paragraphs 36-51, wherein at least one of the AAV vectors is AAV6 or AAV9.
53. The method of any one of paragraphs 36-51, wherein the genetically modified cell is a T cell, hematopoietic stem cell (HSC), macrophage, natural killer cell (NK), or dendritic cell (DC).
54. The method of paragraph 53, wherein the T cell is a CD8+ T cell selected from the group consisting of effector T cells, memory T cells, central memory T cells, and effector memory T cells.
55. The method of paragraph 53, wherein the T cell is a CD4+ T cell selected from the group consisting of Th1 cells, Th2 cells, Th17 cells, and Treg cells.
56. The method of any one of paragraphs 53-55, wherein the cell is bispecific or multispecific.
57. The method of any one of paragraphs 53-56, wherein the cell was isolated from the subject having the disease, disorder, or condition prior to the introduction to the cell.
58. The method of any one of paragraphs 53-56, wherein the cell was isolated from a healthy donor prior to the introduction to the cell.
59. The method of paragraph 57 or 58, wherein the introduction to the cell is performed ex vivo.
60. The method of any one of paragraphs 36-59, wherein the pharmaceutical composition comprises a population of cells derived by expanding the genetically modified cell.
61. The method of any one of paragraphs 34-60, wherein the subject is a human.

### EXAMPLES

### Example 1: AAV-Cpf1 Mediates Efficient Generation of Multiple Knockouts in Human Primary CD4+ T Cells

### Materials and Methods

Generation of LbCpf1 mRNA

Human codon optimized LbCpf1 was from Zetsche, B., et al., Cell. 163(3):759-771 (2015), which was then subcloned into a cDNA in vitro transcription vector. Pseudouridine-modified LbCpf1 mRNA with 5′ cap and poly A tail was generated from the vector at TriLink.

T Cell Culture

Human primary peripheral blood CD4⁺ T cells were acquired from healthy donors (STEMCELL technologies). T cells were cultured in X-VIVO media (Lonza) with 5% human AB serum and recombinant human IL-2 30 U/mL. Before electroporation, T cells were activated with 1:1 ratio of human anti-CD3/anti-CD28 beads (CD3/CD28 Dynabeads, ThermoFisher), which were later removed by magnetic separation rack after two days.

T Cell Electroporation

Electroporation was performed after T cells were activated for 2 days. After using a magnetic holder to remove CD3/CD28 Dynabeads, cells were prepared at a density of 2×10⁵ cells per 10 μL tip reaction or 2×10⁶ cells per 100 μL tip reaction in electroporation Buffer R (Neon Transfection System Kits). T cells were mixed with 1 μg or 10 μg of modified NLS-LbCpf1 mRNA (TriLink) according to reaction volume and electric shocked at program 24 (1,600V, 10 ms and three pulses). After electroporation, the cells were transferred into 1 mL of pre-warmed X-VIVO media (without antibiotics) immediately. Indicated volumes of AAV at a defined multiplicity of infection (MOI, specified in figure legends) were added into the T cells 2-4 hours after electroporation. In some forms, after electroporation, the cells can either be transduced immediately, or after a certain period of time such as, 1 h, 2 h, 4 h, 6 h, 8 h, 12 h, 24 h, 48 h, 72 h, or 96 h.

Western Blot Analysis

Cells were lysed by ice-cold RIPA buffer (Boston BioProducts) containing protease inhibitors (Roche, Sigma) and incubated on ice for 30 minutes. Protein supernatant was collected after centrifugation at 13,000 g at 4° C. for 30 minutes. Protein concentration was determined using the Bradford Protein Assay (Bio-Rad). Protein samples were separated under reducing conditions on 4-15% Tris-HCl gels (Bio-Rad) and analyzed by western blotting using primary antibodies: mouse anti-LbCpf1 (Diagenode 1:3000) followed by secondary anti-rabbit HRP antibodies (Sigma-Aldrich, 1:10,000). Blots were imaged with an Amersham Imager 600.

Construction of AAV Vectors

To generate an AAV crRNA expression vector (AAV-LbcrRNA, or pXD017), the U6-crRNA expression cassette with double BbsI cutting sites was synthesized and subcloned into an AAV backbone containing inverted terminal repeats (ITRs). The LbCpf1 crRNA was designed by Benchling to target the first exon of the TRAC locus and the second exon of PDCD1 (Table 5). Oligonucleotides (Yale Keck) with sticky ends were annealed, phosphorylated and ligated into BbsI-digested vector by T4 ligase (NEB).

TABLE 5
Cpf1 guide sequences used for single and crRNA
array

| Gene name | Spacer Sequence (5′→3′) |
|---|---|
| TRAC 20 nt guide | GAGTCTCTCAGCTGGTACAC |
| | (SEQ ID NO: 1) |
| PDCD1 23 nt guide | GCACGAAGCTCTCCGATGTGTTG |
| | (SEQ ID NO: 20) |
| PDCD1 20 nt guide | GCACGAAGCTCTCCGATGTG |
| | (SEQ ID NO: 2) |

AAV Production and Titration

AAV was produced by transfecting HEK293FT cells (ThermoFisher) in 15-cm tissue culture dishes (Corning). Transfection was done by using AAV2 transgene vectors, packaging (pDF6) plasmid and AAV6/9 serotype plasmid together with polyethyleneimine (PEI). Transfected cells were collected using PBS after post-transfection 72 hours. For the AAV purification, transfected cells were mixed with pure chloroform ( 1/10 volume) and incubated at 37° C. with vigorous shaking for 1 hour. NaCl was added to a final concentration of 1 M, and then centrifuged at 20,000 g at 4° C. for 15 minutes. The chloroform layer was discarded while the aqueous layer was transferred to another tube. PEG8000 was added to 10% (w/v) and shaken until dissolved. The mixture was incubated at 4° C. for 1 hour and then centrifuged at 20,000 g at 4° C. for 15 minutes. The supernatant was discarded and the pellet was suspended in DPBS with MgCl₂, treated with universal nuclease (ThermoFisher) and incubated at 37° C. for 30 minutes. Chloroform (1:1 volume) was then added, shaken and centrifuged at 12,000 g at 4° C. for 15 minutes. The aqueous layer was isolated and concentrated through a 100-kDa MWCO (Millipore). Virus was titered by qPCR using custom Taqman assays (ThermoFisher) targeted to promoter U6.

Flow Cytometry

Surface protein expression was determined by flow cytometry. After electroporation for 5 days, 1×10⁶ cells were incubated with APC-CD4, PE/Cy7-TCR (or PE-TCR) and FITC-CD3 antibodies (Biolegend) for 30 minutes. Stained cells were measured and sorted on BD FACSAria II and analyzed using FlowJo software 9.9.4 (Treestar, Ashland, Oreg.).

Amplicon Sequencing

The resultant PCR products were used for Nextera library preparation following the manufacturer's protocols (Illumina). Briefly, 1 ng of purified PCR product was fragmented and tagged using the Nextera Amplicon Tagment Mix according to the manufacturer's recommendations, followed by limited-cycle PCR with indexing primers and Illumina adaptors. After this amplification, DNA bands were purified with a gel extraction kit (Qiagen). Libraries were sequenced using 100-bp paired-end reads on an Illumina HiSeq 4000 instrument or equivalent, in general generating between 29 to 74 million reads per library. For indel quantification, paired reads were mapped to the amplicon sequences using BWA-MEM with the -M option. 100 bp reads from the SAM file that fully mapped within a +/−75 bp window of expected cut site within the amplicon were then identified (soft-clipped reads discarded). Indel reads were then identified by the presence of “I” or “D” characters within the CIGAR string. Cutting efficiency was quantified as percentage of indels over total (indel plus wild-type) reads within the defined window. Indel variant statistics are provided in Supplementary Dataset 51, where the raw sequencing files are being deposited to SRA.

Standard Statistical Analysis (Non-NGS)

Standard data analysis (Non-NGS) were performed using regular statistics, where NGS data were analyzed with specific pipelines described in Materials and Methods under separate sub-headlines. Data comparison between two groups was performed using a two-tailed unpaired t-test or non-parametric Wilcox test. p values and statistical significance were estimated for all analyses. Prism (GraphPad Software Inc.) and RStudio were used for these analyses.

### Results

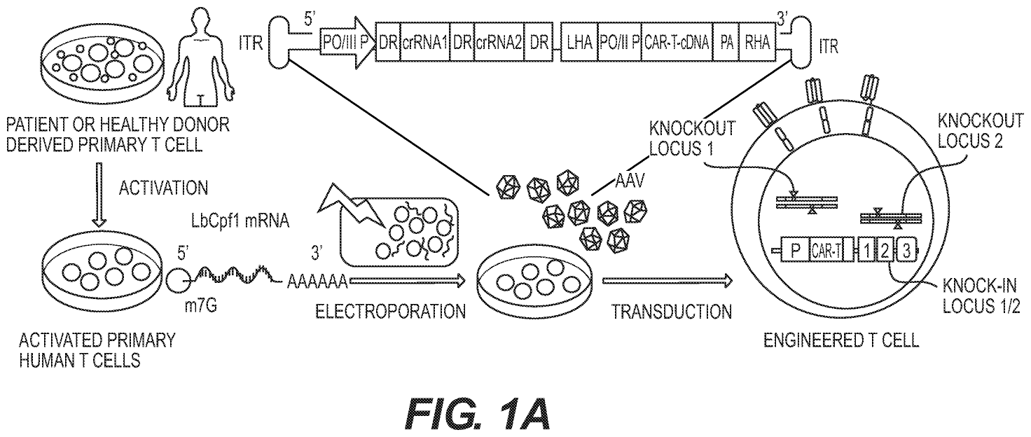

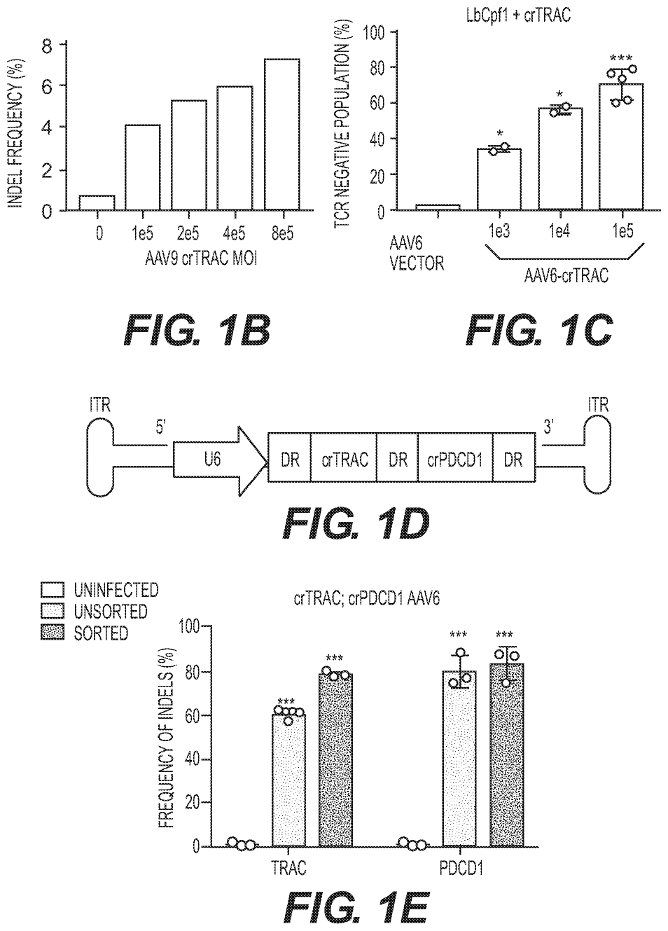

Given the potential of Cpf1 for mammalian cell genome editing and AAV as an effective vehicle for transgene delivery, a workflow of mRNA-AAV introduction for human primary T cell engineering was set up (FIG. 1A). In order to introduce sufficient expression of LbCpf1 (Cpf1 from Lachnospiraceae) and maximize its editing efficiency, a pseudouridine-modified LbCpf1 mRNA with 5′ cap and poly A tail was used according to Li, B., et al., Nat Biomed Eng. 1(5) pii: 0066 (2017). First, a western blot was used to investigate the kinetic expression of LbCpf1 after electroporation. It was observed that the expression of LbCpf1 peaked on day one post-electroporation and diminished around day four. To test the AAV-Cpf1 cutting efficiency in human primary T cells, a guide targeting the 5′ end of the first exon of the TRAC gene was designed and used with two different serotypes of AAV (AAV9 and AAV6) to evaluate cleavage efficiency in human primary CD4⁺ T cells. To confirm successful genomic targeting with AAV-Cpf1 and its functional effect on protein expression in human CD4⁺ T cells, targeted amplicon sequencing (Nextera library prep followed by Illumina sequencing) as well as flow cytometry was used to investigate on-target TCR knockout after AAV-Cpf1 treatment. While Cpf1 mRNA with AAV9-crTRAC generated on-target indels in primary CD4⁺ T cells in a dose-dependent manner (FIG. 1B), AAV6-crTRAC yielded a much higher knockout efficiency (FIG. 1C), where on average a 70.36% knockout efficiency was achieved with a single transduction at multiplicity of infection (MOI) of 1e5 (FIG. 1C).

Using a single customized CRISPR array, Cpf1 can introduce multiple mutations in various mammalian cell types (Zetsche, B., et al, Nature biotechnology. 35(1):31-34 (2017)). It was then explored whether the AAV-Cpf1 system could achieve highly efficient multiplex genome editing in human primary T cells by AAV6 delivery of a single crRNA array. A new AAV vector was constructed which delivered a U6-promoter-driven Cpf1 array targeting the TRAC and PDCD1 genes (crTRAC;crPDCD1) (FIG. 1D). It was observed that one transduction simultaneously generated efficient editing in both loci of primary CD4+ T cells. Quantification by Nextera library preparation and Illumina sequencing showed that the mutation frequencies at TRAC and PDCD1 loci in unsorted cells reached a bulk efficiency of 60.39% and 80.07%, respectively (FIG. 1E), which was further enriched by FACS sorting on the TCR⁻ population (78.80% and 83.63%, respectively) (FIG. 1E). Together, these results demonstrated that AAV6 delivery of crRNA in combination with LbCpf1 mRNA electroporation is an effective means to edit multiple loci in human primary CD4+ T cells.

### Example 2: AAV-Cpf1 Mediates Simultaneous Multiplex Knock-Ins and Knockouts in Human Primary CD4+ T Cells

### Materials and Methods

Construction of AAV Vectors

An AAV crRNA expression vector (AAV-LbcrRNA, or pXD017) containing the U6-crRNA expression cassette with crRNAs targeting the first exon of the TRAC locus and the second exon of PDCD1 was generated as described in Example 1. To generate the HDR construct, the left and right homologous arms of the TRAC or PDCD1 locus were amplified by PCR from primary CD4⁺ T cells using locus-specific primer sets HDR-F1/R1 and HDR-F2/R2 (Table 6). For transgene cloning, the HDR-R1 and HDR-F2 were connected with a multiple cloning site (MCS) (Table 6). Homologous donor templates were cloned into the AAV-LbcrRNA with or without a crRNA. For generation of the HDR template, the EFS-dTomato-PA cassette was cloned into the multi-clone site (MCS).

TABLE 6
PCR primers for HDR AAV vector construction

| Primer name | Sequence (5′→3′) |
|---|---|
| TRAC HDR F1 | TCAACTAGATCTTGAGACAAGGTACGATGTAAGGA |
| | GCTGCTGTGACT (SEQ ID NO: 4) |
| TRAC HDR R1 | GGTACCTCGAGCGTACGGGTCAGGGTTCTGGATAT |
| (With MCS) | CTGTG (SEQ ID NO: 5) |
| TRAC HDR F2 | CGTACGCTCGAGGTACCGAGAGACTCTAAATCCAG |
| (With MCS) | TGACAAG (SEQ ID NO: 6) |
| TRAC HDR R2 | CTTTTATTAAGCTTGATATCGAATTGTGGGTTAAT |
| | GAGTGACTGCG (SEQ ID NO: 7) |
| PDCD1 HDR F1 | TGGCAGGAGAGGGCACGTGGGCAGCCTCACGTAGA |
| | AGGAA (SEQ ID NO: 8) |
| PDCD1 HDR R1 | TCCGAGAATTCTTTGTTAACTGTGTTGGAGAAGCT |
| (With MCS) | GCAGGT (SEQ ID NO: 9) |
| PDCD1 HDR F2 | CACAGTTAACAAAGAATTCTCGGAGAGCTTCGTGC |
| (With MCS) | TAAACTGG (SEQ ID NO: 10) |
| PDCD1 HDR R2 | GCGGCCGCTCGGTCCGCACCTGATCCTGTGCAGGA |
| | GGG (SEQ ID NO: 19) |

TABLE 7
The sequences of HDR arms
HDR Template

| name | Sequence (5′→3′) |
|---|---|
| TRAC Left Arm | gatgtaaggagctgctgtgacttgctcaaggccttatatcgagtaaacggtagcgctggggctt |
| | agacgcaggtgttct |
| | gatttatagttcaAAACCTCTATCAATGAGAGAGCAATCTCCTGGTAATGTGATAGATTTCCCAA |
| | CTTAATGCCAACATACCATAAACCTCCCATTCTGCTAATGCCCAGCCTAAGTTGGGGAGAC |
| | CACTCCAGATTCCAAGATGTACAGTTTGCTTTGCTGGGCCTTTTTCCCATGCCTGCCTTTACT |
| | CTGCCAGAGTTATATTGCTGGGGTTTTGAAGAAGATCCTATTAAATAAAAGAATAAGCAGT |
| | ATTATTAAGTAGCCCTGCATTTCAGGTTTCCTTGAGTGGCAGGCCAGGCCTGGCCGTGAAC |
| | GTTCACTGAAATCATGGCCTCTTGGCCAAGATTGATAGCTTGTGCCTGTCCCTGAGTCCCA |
| | GTCCATCACGAGCAGCTGGTTTCTAAGATGCTATTTCCCGTATAAAGCATGAGACCGTGAC |
| | TTGCCAGCCCCACAGAGCCCCGCCCTTGTCCATCACTGGCATCTGGACTCCAGCCTGGGTT |
| | GGGGCAAAGAGGGAAATGAGATCATGTCCTAACCCTGATCCTCTTGTCCCACAGATATCCA |
| | GAACCCTGACC (SEQ ID NO: 26) |
| TRAC Right | GAGAGACTCTAAATCCAGTGACAAGTCTGTCTGCCTATTCACCGATTTTGATTCTCAAACAA |
| Arm | ATGTGTCACAAAGTAAGGATTCTGATGTGTATATCACAGACAAAACTGTGCTAGACATGA |
| | GGTCTATGGACTTCAAGAGCAACAGTGCTGTGGCCTGGAGCAACAAATCTGACTTTGCAT |
| | GTGCAAACGCCTTCAACAACAGCATTATTCCAGCAGACACCTTCTTCCCCAGCCCAGGTAA |
| | GGGCAGCTTTGGTGCCTTCGCAGGCTGTTTCCTTGCTTCAGGAATGGCCAGGTTCTGCCCA |
| | GAGCTCTGGTCAATGATGTCTAAAACTCCTCTGATTGGTGGTCTCGGCCTTATCCATTGCCA |
| | CCAAAACCCTCTTTTTACTAAGAAACAGTGAGCCTTGTTCTGGCAGTCCAGAGAATGACAC |
| | GGAAAAAAAGCAGATGAAGAGAAGGTGGCAGGAGAGGGCACGTGGCCCAGCCTCAGTC |
| | TCTCCAACTGAGTTCCTGCCTGCCTGCCTTTGCTCAGACTGTTTGCCCCTTACTGCTCTTCTA |
| | GGCCTCATTCTAAGCCCCTTCTCCAAGTTGCCTCTCCTTATTTCTCCCTGTCTGCCAAAAAAT |
| | CTTTCCCAGCTCACTAAGTCAGTCTCACGCAGTCACTCATTAACCCAC (SEQ ID NO: 27) |
| PDCD1 Left | GCAGCCTCACGTAGAAGGAAGAGGCTCTGCAGTGGAGGCCAGTGCCCATCCCCGGGTGG |
| Arm | CAGAGGCCCCAGCAGAGACTTCTCAATGACATTCCAGCTGGGGTGGCCCTTCCAGAGCCC |
| | TTGCTGCCCGAGGGATGTGAGCAGGTGGCCGGGGAGGCTTTGTGGGGCCACCCAGCCCC |
| | TTCCTCACCTCTCTCCATCTCTCAGACTCCCCAGACAGGCCCTGGAACCCCCCCACCTTCTCC |
| | CCAGCCCTGCTCGTGGTGACCGAAGGGGACAACGCCACCTTCACCTGCAGCTTCTCCAACA |
| | CA (SEQ ID NO: 28) |
| PDCD1 Right | TCGGAGAGCTTCGTGCTAAACTGGTACCGCATGAGCCCCAGCAACCAGACGGACAAGCTG |
| Arm | GCCGCCTTCCCCGAGGACCGCAGCCAGCCCGGCCAGGACTGCCGCTTCCGTGTCACACAA |
| | CTGCCCAACGGGCGTGACTTCCACATGAGCGTGGTCAGGGCCCGGCGCAATGACAGCGG |
| | CACCTACCTCTGTGGGGCCATCTCCCTGGCCCCCAAGGCGCAGATCAAAGAGAGCCTGCG |
| | GGCAGAGCTCAGGGTGACAGGTGCGGCCTCGGAGGCCCCGGGGCAGGGGTGAGCTGAG |
| | CCGGTCCTGGGGTGGGTGTCCCCTCCTGCACAGGATCAG (SEQ ID NO: 29) |

Surveyor (T7E1) Assay

Genomic DNA was collected using the QuickExtract DNA Extraction Solution (Epicentre) after electroporation for 5 days. Target loci from human T cell genomic DNA were amplified using appropriate primers (Table 8). The PCR products were gel-purified using QIAquick Gel Extraction Kit from 2% E-gel EX and quantified. After purification, 200 ng of purified PCR product was denatured, annealed, and digested with T7E1 (New England BioLabs). The digested PCR products were loaded into 2% E-gel EX, and the amount of DNA fragments were quantified using E-Gel™ Low Range Quantitative DNA Ladder (ThermoFisher).

TABLE 8
PCR primers for T7E1 assay

| Primer | | |
|---|---|---|
| name | Forward (5′→3′) | Reverse (5′→3′) |
| TRAC | CTGAGTCCCAGTCCATC | AGGGTTTTGGTGGCAAT |
| | ACG (SEQ ID NO: 12) | GGA (SEQ ID NO: 13) |
| PDCD1 | GTAGGTGCCGCTGTCAT | GAGCAGTGCAGACAGGA |
| | TGC (SEQ ID NO: 14) | CCA (SEQ ID NO: 15) |

Analysis of HDR by In-Out PCR

A semi-quantitative In-Out PCR was performed to measure the rates of dTomato integration at the TRAC locus as previously described (Wang, J., et al., Nucleic Acids Res., 44(3):e30 (2016)). The assay used three primers in one PCR reaction. One primer recognizes a sequence contained in the dTomato cassette; a second primer binds to genomic sequence outside of this AAV donor; the third primer binds to a sequence of the left TRAC homology arm (Table 9). This PCR product, designated TRAC-HDR, was normalized by comparison to the product resulting from the control with genomic DNA isolated from normal human CD4⁺ T cells.

TABLE 9
PCR primers for In-Out PCR assay

| Primer name | Forward (5′→3′) |
|---|---|
| TRAC 1st | CCCTTGTCCATCACTGGCAT |
| (Left arm) | (SEQ ID NO: 16) |
| TRAC 2st (Genomic | GCACACCCCTCATCTGACTT |
| sequence outside | (SEQ ID NO: 17) |
| of AAV donor) | |
| dTomato 3rd | AACACAGGACCGGTTCTAGACGTACGGCCA |
| | CCATGGTGAGCAAGGGCGAG |
| | (SEQ ID NO: 25) |
| CD22CAR 3rd | GAAATCAAAGCGGCCGCAG |
| | (SEQ ID NO: 18) |

Flow Cytometry

Flow cytometry was performed as described in Example 1.

Amplicon Sequencing

Targeted amplicon capture and next-generation sequencing (NGS) was performed as described in Example 1.

HDR Mapping

For HDR quantification, FASTQ reads were mapped to possible amplicons based on primer combinations and HDR status. Mapping was performed for full amplicons and for “informative” amplicons, which were truncated so that 100 bp reads would have at least 20 bp homology with the CAR sequence (or with the other TRAC arm, in the case of wild-type sequences). Informative reads would be used to distinguish wild-type, NHEJ, and HDR reads with higher confidence. Paired reads were mapped to amplicon sequences using BWA-MEM with -M flag to generate SAM files. SAMtools was used to convert files to BAM, sort, index, and generate summary statistics of read counts with the idxstats option. To quantify wild-type vs NHEJ reads, reads that mapped to “info_nonHDR” sequence (described below) were taken, reads with indels (I″ or “D” characters within the CIGAR string) were called as NHEJ. Otherwise, reads were called as wild-type. Read counts were then pooled for downstream analysis. Description of amplicon sequences are provided below:

amplicon_nonHDR: refers to full amplicon from F1 and R1 of genomic, wild-type DNA.

amplicon_CAR_F1: refers to full amplicon from F1 and R1 of expected, integrated CAR.

amplicon_CAR_F2: refers to full amplicon from F2 (primer site within the MCS as opposed to outside) and R1 of expected, integrated CAR.

info_nonHDR: same as amplicon_nonHDR, except truncated to 80 bp of the TRAC arms.

info_CAR_F1: same as amplicon_CAR_F1, except truncated to 80 bp of the TRAC arms flanking the TRAC-CAR interface.

info_CAR_F2: same as amplicon_CAR_F2, except truncated to 80 bp of the TRAC arms flanking the TRAC-CAR interface (relevant to the right arm only, since F2 is within the CAR sequence).

HDR, NHEJ and WT scores were calculated as follows:

info_nonHDR=info_WT+info_NHEJ

hdr_score=info_CAR_F2/(info_CAR_F2+info_nonHDR)

wt_score=info_WT/(info_CAR_F2+info_nonHDR)

nhej_score=info_NHEJ/(info_CAR_F2+info_nonHDR)

### Results

Due to various technical hurdles including the size of Cas9 transgene and protein, the dependence on tracrRNA for the Cas9 system, and previously conceived low targeting efficiency by Cpf1, one-step generation of precise knock-ins of multiple large functional gene cassettes and simultaneous knockouts of two or more genes in primary T cells remains challenging. To overcome this, in addition to using chemically modified LbCpf1 mRNA, the HDR template was cloned into the AAV6-crRNA array vector, thereby allowing simultaneous delivery of the homologous donor template as well as a multi-loci targeting single crRNA array.

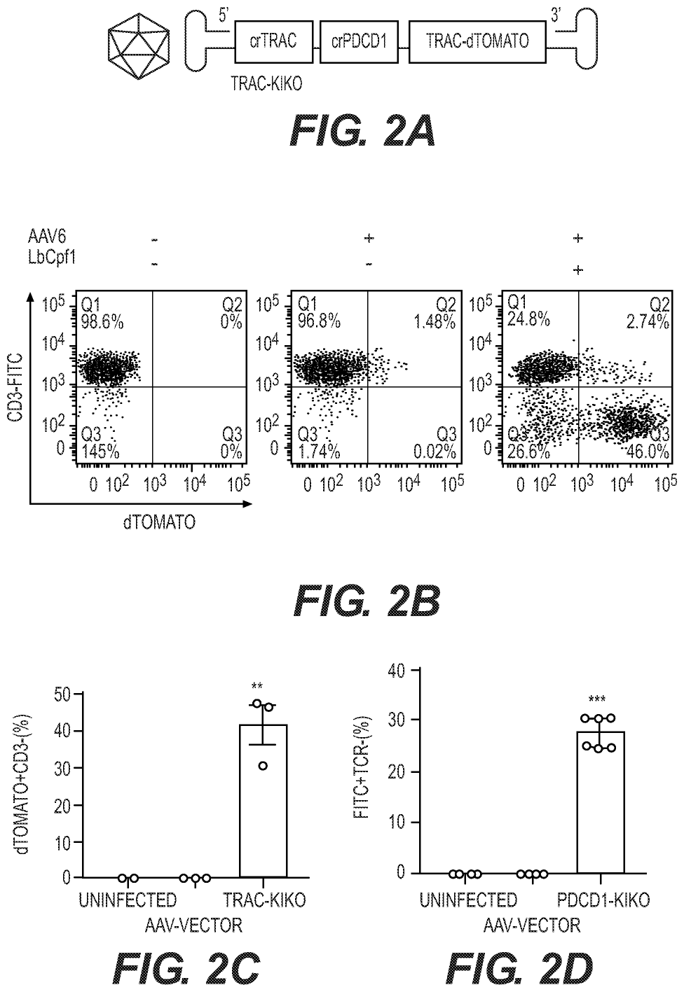

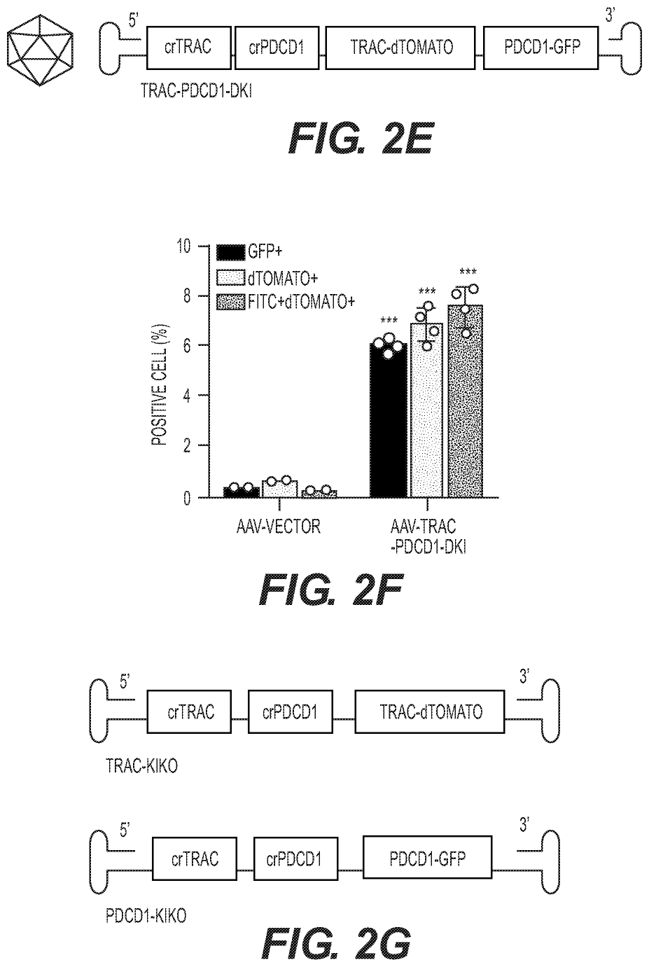

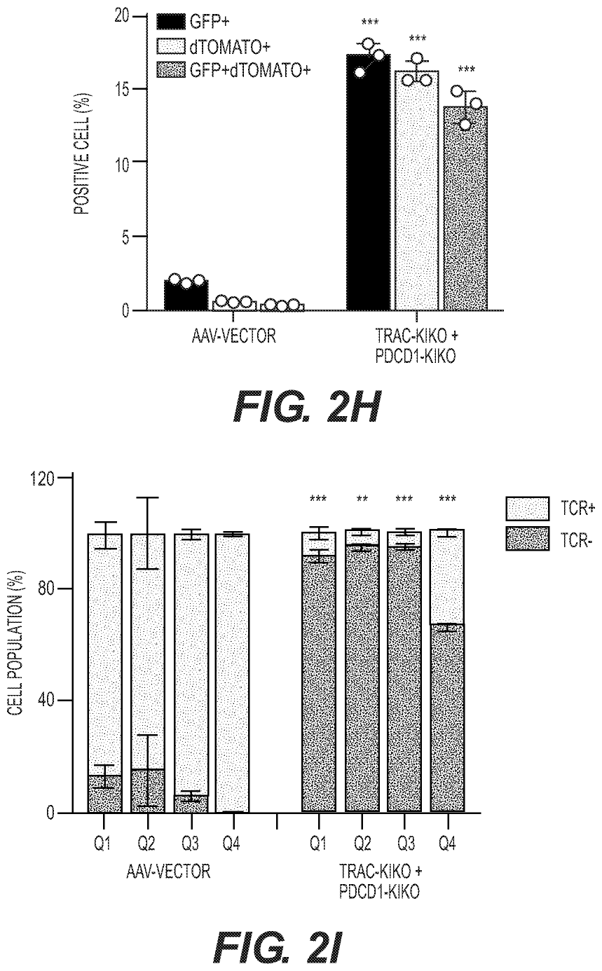

The simultaneous knock-in of a transgene expression and knockout of an additional gene was first tested; a reporter gene (dTomato) driven by an EFS promoter was targeted between TRAC homology arms, with a crRNA opening the double stranded DNA, and a second crRNA in the same array knocking out PDCD1, termed PDCD1^{KO};dTomato-TRAC^{KI} (TRAC-KIKO for short) (FIG. 2A). Five days after joint electroporation and AAV transduction, TRAC-KIKO mediated efficient, targeted dTomato integration as measured by flow cytometry (FIG. 2B). CD3 and TCR can form a complex on the cell surface, thus TCR knockout efficiency can be determined by staining of CD3 (Torikai, H., et al., Blood., 119(24):5697-705 (2012)). Greater than 70% of CD4⁺ T cells lost expression of CD3, with on-target integration of dTomato at greater than 40% of total treated cells (FIGS. 2B-C). To confirm the integration of the dTomato in the genome at the DNA level, a semi-quantitative In-Out PCR was first used to measure the rates of dTomato integration at the TRAC locus. It was observed that the bulk HDR efficiency reached 34.7% in unsorted cells, and was further enriched to 69.5% in CD3⁻dTomato⁺ sorted cells by gel quantification. Mapping AAV6 vector integration by targeted amplicon capture and next-generation sequencing (NGS) revealed the HDR junctions and confirmed the quantitative results (42.18% and 72.84% HDR in bulk and enriched, respectively). A T7E1 assay was also used to survey the PDCD1 knockout efficiency. Indels at the predicted cleavage sites at bulk frequencies of 47% (Nextera 62.34%) and 72% (Nextera 87.03%) were observed in unsorted and TCR⁻ dTomato⁺ sorted cells. The T7E1 result is likely an underestimate of the indel efficiency due to the fact that high frequency homoduplex mutants are insensitive to T7E1 (Kim, H., et al., Nat. Methods., 8(11):941-3 (2011)). To test knock-in at sites other than TRAC, another KIKO vector was generated: TRAC^{KO};GFP-PDCD1^{KI} (PDCD1-KIKO for short) which mediates combinatorial TRAC knockout and GFP transgene knock-in into the PDCD1 locus. This vector contains a crRNA array including crRNAs targeting the PDCD1 and TRAC genes and a cassette with a GFP reporter driven by an EFS promoter is inserted between the PDCD1 homology arms. Five days after electroporation and transduction of AAV-Cpf1 PDCD1-KIKO, the knock-in and knockout efficiency was examined by flow cytometry. As shown in FIG. 2D, over 80% of CD4⁺ T cells lost expression of the TCR, with stable GFP addition detected in nearly 30% of the cells.

The capacity to undergo double knock-ins is essential for multi-feature CAR-T, such as bi-specifics. In order to enable double knock-in to the same T cell, an AAV vector was first generated, designated dTomato-TRAC^{KI};GFP-PDCD1^{KI} (TRAC-PDCD1-DKI for short), where dTomato and GFP are targeted for integration into the TRAC locus and PDCD1 locus respectively (FIG. 2E). 5 days after LbCpf1 mRNA electroporation and AAV TRAC-PDCD1-DKI transduction of human primary T cells, a population of double positive GFP⁺dTomato⁺ T cells was produced at 7.54%, along with 5.97% GFP single positive and 6.82% dTomato single positive populations (FIG. 2F). Considering that two HDR templates in a single AAV vector might compete with each other, an alternative strategy was developed. Two different AAV vectors (PDCD1^{KO};dTomato-TRAC^{KI} and TRAC^{KO};GFP-PDCD1^{KI}) were used for dual-targeting, which shared the same crRNA array but contained different HDR templates (FIG. 2G). Flow cytometry results showed that compared to the single AAV method, the two-AAV system templates had higher integration efficiency for generation of both double positive and single positive knock-ins, which produced on average 13.83% GFP⁺dTomato⁺, 17.23% GFP⁺ and 16.17% dTomato⁺ T cells (FIG. 2H). The TCR expression levels in different subpopulations were also analyzed by FACS. It was observed that all the T cells that underwent integration (including single and double positives, Q1, Q2 and Q3) almost completely lost the TCR expression, and 65% of cells that did not undergo integration (GFP⁻dTomato⁻, Q4) lost TCR expression; whereas vector transduced T cells mostly retained intact TCR (FIG. 2I). These data indicate that efficient and precise double knock-ins in human T cells can be achieved by AAV-Cpf1 using either an all-in-one AAV, or two AAVs with different donors.

### Example 3: One-Step Generation of CAR T Cells with Anti-CD22 CAR Knock-in at the TRAC Locus and Simultaneous PDCD1 Disruption by AAV-Cpf1 KIKO

### Materials and Methods

Cell culture, mRNA electroporation, AAV transduction, Nextera—Illumina sequencing, and the T7E1 assay were performed as previously described in Examples 1 and 2.

Construction of AAV Vectors

An AAV crRNA expression vector (AAV-LbcrRNA, or pXD017) containing the U6-crRNA expression cassette with crRNAs targeting the first exon of the TRAC locus and the second exon of PDCD1 was generated as described in Example 1. To generate the HDR construct, the left and right homologous arms of the TRAC or PDCD1 locus were amplified by PCR using locus-specific primer sets HDR-F1/R1 and HDR-F2/R2 from primary CD4⁺ T cells. For transgene cloning, the HDR-R1 and HDR-F2 were connected with a multiple cloning site (MCS). Homologous donor templates were cloned into the AAV-LbcrRNA with or without a crRNA. The generation of CD22BBz CAR was previously described (Haso, W., et al., Blood., 121(7):1165-74 (2013)). Briefly, the CAR comprises a single chain variable fragment CD22 binding scFV (m971) specific for the human CD22 followed by CD8 hinge-transmembrane-regions linked to 4-1BB (CD137) intracellular domains and CD3ζ intracellular domain. Based on a pXD017-dTomato backbone, the m971-BBz was cloned into this vector using a gBlock (IDT). For generation of the HDR template, the EFS-CAR22BBz-PA cassette was cloned into the multi-clone site (MCS).

Flow Cytometry

Surface protein expression was determined by flow cytometry. After electroporation for 5 days, 1×10⁶ cells were incubated with APC-CD4, PE/Cy7-TCR (or PE-TCR) and FITC-CD3 antibodies (Biolegend) for 30 minutes. For the CD22BBz CAR transduced T cells were incubated with 0.2 μg CD22-Fc (R&D system) in 100 μL PBS for 30 minutes, and then stained with PE-IgG-Fc (Biolegend). After washing twice, the stained cells were measured and sorted on BD FACSAria II, and analyzed using FlowJo software 9.9.4 (Treestar, Ashland, Oreg.).

Analysis of HDR by In-Out PCR

A semi-quantitative In-Out PCR was performed to measure the rates of CAR22 m971-BBz integration at the TRAC locus as previously described (See Example 2). Briefly, three primers were used in one PCR reaction. One primer recognizes a sequence contained in the m971-BBz cassette; a second primer binds to genomic sequence outside of this AAV donor; the third primer binds to a sequence of the left TRAC homology arm. This PCR product, designated TRAC-HDR, was normalized by comparison to the product resulting from the control with genomic DNA isolated from normal human CD4⁺ T cells.

### Results

Recent studies have demonstrated that integration of CAR into the TRAC locus can potentiate anti-CD19 CAR effects in leukemia, and specific genetic knockouts in CART cells could reduce T cell exhaustion (Eyquem, J., et al., Nature. 543(7643):113-117 (2017); Rafiq, S., et al., Nat. Biotechnol., 36(9):847-856 (2018); Ren, J., et al., Clin. Cancer Res., 23(9):2255-2266 (2017)). CD22-CAR targeting B-cell precursor acute lymphoblastic leukemia was safe and provided high response rates for pediatric patients who had failed chemotherapy and/or a CD19-targeted CAR T cell treatment (Haso, W., et al., Blood., 121(7):1165-74 (2013); Fry, T J., et al., Nat. Med., 24(1):20-28 (2018)).

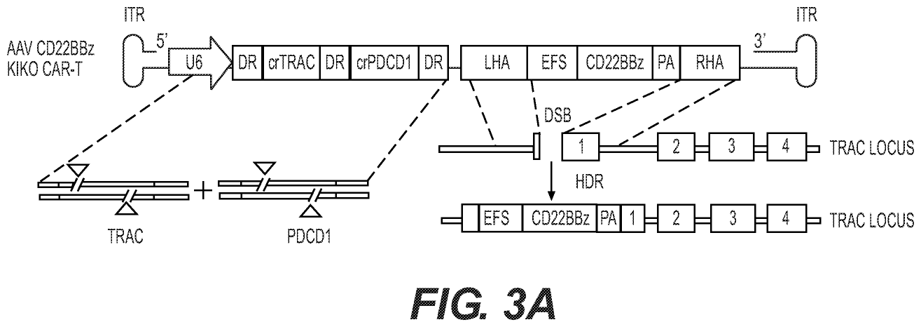

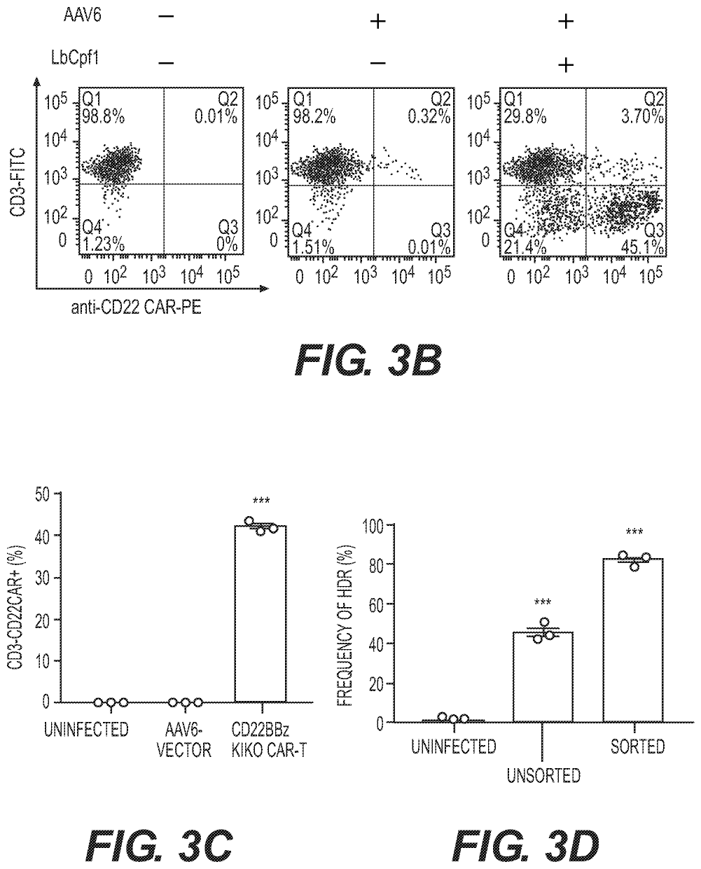

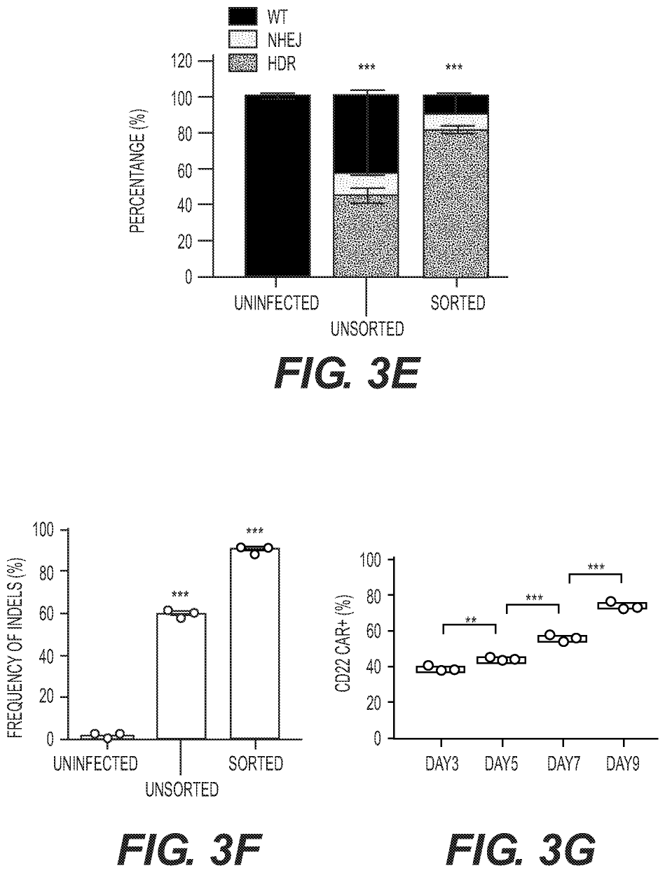

To evaluate whether this technology could be applied to therapeutically-relevant CAR-T gene editing, a single AAV construct, designated PDCD1^{KO};CD22BBz-TRAC^{KI} (CD22BBz KIKO or Cpf1 CAR22 for short) (FIG. 3A), was generated for delivering a double-targeting crRNA array and an HDR template, mediating a CD22-specific CAR integration into the TRAC locus with PDCD1 knockout in human primary T cells. The HDR template contains an EFS-CD22BBz-PolyA cassette, where the CD22BBz CAR transgene is driven by an EFS promoter and terminated by a short polyA, flanked by two arms homologous to the TRAC locus (FIG. 3A).

Five days after electroporation and transduction, AAV-Cpf1 with CD22BBz KIKO generated precisely targeted knock-ins and knockouts (FIGS. 3B-C) with limited toxicity and high viability (not shown). With stimulation, the electroporated T cells quickly expanded over the course of the 26 days observed. Specifically, a bulk population of 66.5% of CD4⁺ T cells had endogenous TCR knocked out (FIG. 3B), and 44.6% of these TCRs were replaced by the CD22BBz CAR as measured by flow cytometry (FIG. 3C). To confirm the integration of the CD22BBz CAR at the genomic level, semi-quantitative In-Out PCR as well as Nextera—Illumina sequencing was used to measure the rates of CD22BBz integration at the TRAC locus. As shown in FIG. 3D, the bulk HDR efficiency with single transduction reached 45.46% in unsorted cells and enriched to 81.88% in CD3⁻CAR⁺ sorted cells. The non-homologous end joining (NHEJ) variants of TRAC were observed at 13.01% in the bulk population and 9.97% in the sorted population on average (FIG. 3E). The T7E1 assay and Nextera—NGS were used to survey the PDCD1 knockout efficiency. A high efficiency of knockouts was observed, showing an average of 59.73% indels in bulk population and 90.39% in CD3⁻CAR⁺ sorted T cells (FIG. 3F). No mutation was found at uninfected control, indicating a clean background. The fraction of CAR22⁺ T cells steadily increased over time, starting at 38.73% on day 3 and ramping up to 74.13% on day 9 after stimulation with target cells (FIG. 3G). The increase of the percentage of CD22BBz⁺ CAR-T cells was likely due to the negative selection of non-functional cells. These data demonstrated a simple and rapid method to generate targeted knock-in CARs with simultaneous immune checkpoint regulator knockout at high efficiency using the AAV-Cpf1 KIKO system in one-step.

### Example 4: AAV-Cpf1 KIKO Derived CAR-T Cells Outperform Lentiviral CAR T Cells

### Materials and Methods

Cell culture, mRNA electroporation, and AAV vector construction and transduction were performed as previously described in Examples 1-3.

Flow Cytometry

Flow cytometry was performed as described in the previous Examples. For the CD22BBz CAR, transduced T cells were incubated with 0.2 μg CD22-Fc (R&D system) in 100 μL PBS for 30 minutes, and then stained with PE-IgG-Fc (Biolegend). For the T cell exhaustion assay, T cells (e.g., T-cells modified by AAV or lentivirus) were co-cultured with NALM6 cells at 1:1 E:T ratio for 3 days. 1×10⁶ cells were incubated with 0.2 μg CD22-Fc (R&D Systems) in 100 μL PBS for 30 minutes and then stained with PE-IgG-Fc, PD-1-FITC, TIGIT-APC and LAG3-Percp/cy5.5 (Biolegend) for 30 minutes. After washing twice, the stained cells were measured and sorted on BD FACSAria II, and analyzed using FlowJo software 9.9.4 (Treestar, Ashland, Oreg.).

Generation of Lentiviral CD22BBz CAR T Cells

The EFS1α-CAR22BBz-PA cassette was cloned into a lentiviral vector, making Lenti-EFS1α-CAR22BBz-PA (pXD039). For lentivirus production, HEK293FT cells were plated in 15 cm dishes the night before transfection. Cells were transfected with lentiviral vector, pSPAX2 and pMD2.G packaging plasmids at a ratio of 4:3:2 using the Polyethylenimine (PEI) reagent. Transfection media was changed with fresh media (DMEM with 10% FBS and 1% penicillin/streptromycin). After transfection for 48 hours, the viral supernatant was collected, filtered and concentrated by ultracentrifugation at 25,000 rpm for 90 minutes 4° C. in 70-Ti rotor. The viral pellet was then resuspended at 100× in cold PBS and stored at −80° C. T cells for viral infection were activated similarly to T cell electroporation. After stimulation for 48 hours, T cells were infected with 2× concentrated virus by spinoculation in retronectin-coated (Takara) plates at 800 g for 45 minutes at 32° C. Control mock-transduced T cells were also generated in the same way.

Generation of Stable Cell Lines

Lentivirus including GFP-luciferase reporter genes were produced as previously described by Chen, et al., Cell, 160(6):1246-1260 (2015). NALM6 cells (ATCC) were infected with 2× concentrated lentivirus by spinoculation in retronectin-coated (Takara) plates at 800 g for 45 minutes at 32° C. After infection for 2 days, the GFP positive cells (NALM6-GL) were sorted on a BD FACSAria II. The second round sorting was performed after culture for two additional days. To test the luciferase expression in NALM6-GL, cells were incubated with 150 μg/ml D-Luciferin (PerkinElmer) and intensity of bioluminescence was measured by an IVIS system.

Intracellular Staining of IFNγ and TNF-α

Intracellular flow cytometry was performed to detect the expression level of IFNγ and TNF-α. After infection for 4 days, AAV transduced CD22BBz CAR and Lenti-CD22BBz CAR T cells were co-cultured with NALM6 in fresh media which was supplied with brefeldin A and 2 ng/mL IL-2. After being incubated for 5 hours, T cells were collected and stained for surface CAR first. After membrane protein staining, cells were fixed and permeabilized by fixation/permeabilization solution (BD), followed by addition of anti-IFNγ-APC or anti-TNF-α-FITC for intracellular staining After 30 mins, the stained cells were washed by BD Perm/Wash™ buffer and measured by BD FACSAria II.

Cancer Cell Killing Assay

2×10⁴ NALM6-GL cells were seeded in a 96 well plate. The modified or control T cells were co-cultured with NALM6-GL at indicated E:T ratios for 24 hours. Cell proliferation was tested by adding 150 μg/ml D-Luciferin (PerkinElmer) into each well. After 5 minutes, luciferase assay intensity was measured by a plate reader (PerkinElmer).

### Results

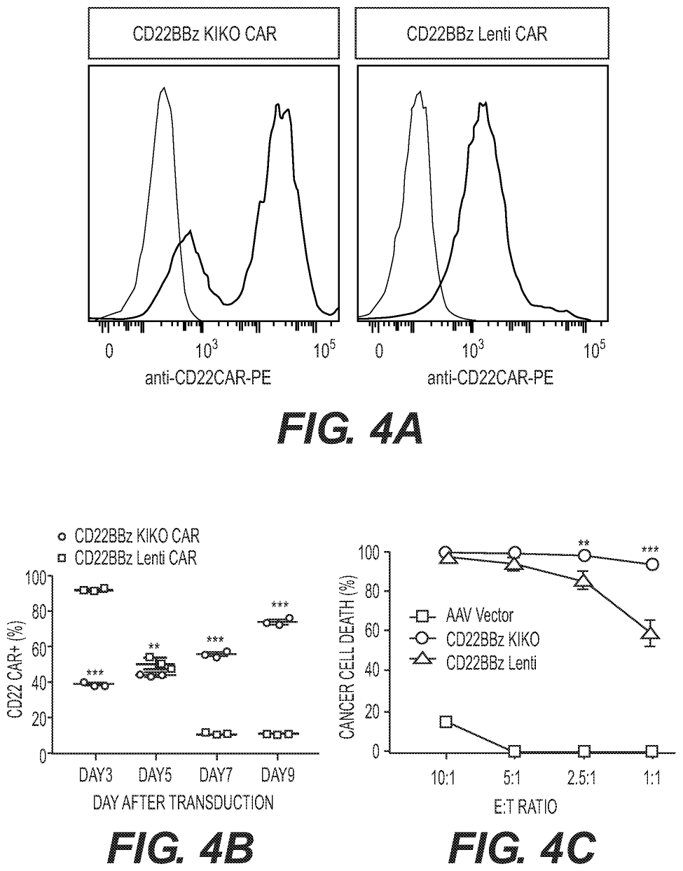

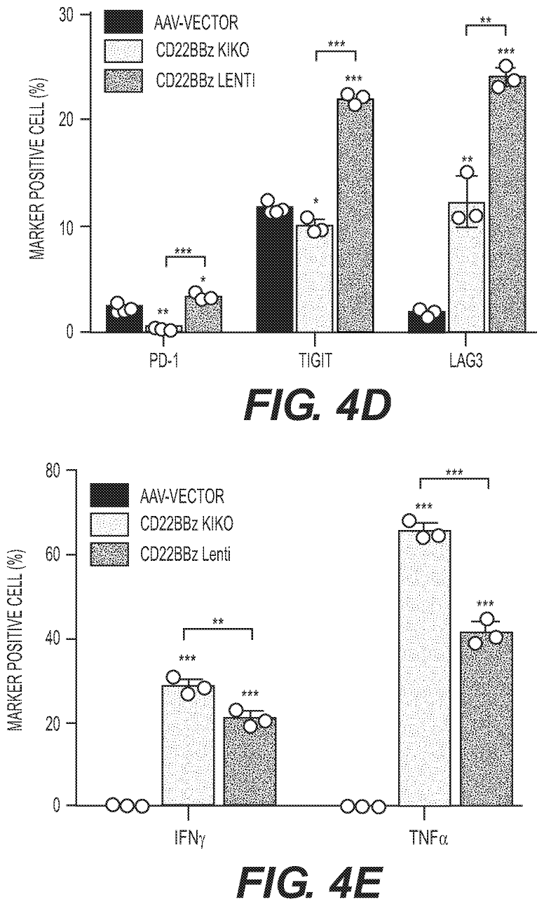

Using functional assays, the characteristics of AAV-Cpf1 KIKO CAR-T were evaluated in comparison to lentiviral CAR-T. Flow cytometry analysis showed that the CD22BBz KIKO generated bulk CAR-T cells with a higher-level bimodal pattern of CAR transgene expression (clear CAR⁺ vs. CAR⁻ populations), compared to CD22BBz Lenti CAR transduced T cells that have a continuous pattern (mixture of CARP vs. CAR⁻ populations) (FIG. 4A). Time-course analysis of CAR transgene retention showed that KIKO CAR-T exhibited a steadily increasing population of CD22CAR⁺ cells after transduction, whereas the Lenti-CD22BBz transduced human T cells showed a decreasing fraction of CD22CAR⁺ cells (FIG. 4B). Starting day 7 post transduction, the CAR expression levels in bulk AAV-Cpf1 transduced T cells were significantly higher than those in lentivirally transduced cells (FIG. 4B).

The ability of CAR T cells to kill cognate cancer cells was evaluated using co-culture (kill assay). The cytotoxic activity of CD22CAR⁺ T cells against NALM6-GL target cells (stably transduced with GFP and Luciferase transgenes) at different effector:target (E:T) ratios was determined. The results demonstrated that while killing was saturated or near-saturated at 10:1 or 5:1 E:T ratio, KIKO CD22BBz CAR showed significantly higher killing ability at 2.5:1 and 1:1 E:T ratios compared to Lenti-CD22BBz CAR (FIG. 4C). In fact, the KIKO CD22BBz CAR demonstrated a relatively steady killing ability across all the tested E:T ratios (all >90% cancer cell death), whereas the Lenti-CD22BBz CAR rapidly lost killing ability as the E:T ratio decreased (FIG. 4C).

Since effector cells such as CAR-T often undergo exhaustion, the T cell exhaustion markers including PD-1, TIGIT and LAG-3 were examined. Comparison of AAV-Cpf1 KIKO CAR-T vs. lentiviral CAR-T showed that the expression of all three markers were significantly lower in KIKO CD22BBz CAR-T than that of lentiviral CAR-T (FIG. 4D; PD1 group: Vector vs. PDCD1^{KO}; CD22BBz-TRAC^{KI},** p=0.0014; Vector vs. lentiviral CAR-T, * p=0.0489; PDCD1^{KO};CD22BBz-TRAC^{KI} vs. lentiviral CAR-T, *** p<0.001; TIGIT group: Vector vs. PDCD1^{KO};CD22BBz-TRAC^{KI},* p=0.0254; Vector vs. lentiviral CAR-T, *** p<0.001; PDCD1^{KO};CD22BBz-TRAC^{KI} vs. lentiviral CAR-T, *** p<0.001; LAG3 group: Vector vs. PDCD1^{KO}; CD22BBz-TRAC^{KI}, ** p=0.0017; Vector vs. lentiviral CAR-T, *** p<0.001; PDCD1_{KO};CD22BBz-TRAC^{KI} vs. lentiviral CAR-T, ** p=0.015).

Furthermore, production of effector cytokines by KIKO CAR-T and lentiviral CAR-T was directly measured. Quantitative analysis by flow cytometry demonstrated that the KIKO CAR-T showed significantly higher IFNγ and TNF-α production compared to lentiviral CAR-T (FIG. 4E; IFNγ group: Vector vs. PDCD1^{KO};CD22BBz-TRAC^{KI},*** p<0.001; Vector vs. lentiviral CAR-T, ***p<0.001; PDCD1^{KO};CD22BBz-TRAC^{KI} vs. lentiviral CAR-T, **p=0.006; TNF-α group: Vector vs. PDCD1^{KO};CD22BBz-TRAC^{KI},*** p<0.001; Vector vs. lentiviral CAR-T, *** p<0.001; PDCD1^{KO};CD22BBz-TRAC^{KI} vs. lentiviral CAR-T, *** p<0.001).

These experiments together demonstrate that the KIKO CAR targeting method generates engineered CAR-T cells with superior effector function and reduced levels of exhaustion without compromising the simplicity of transgene delivery, making the AAV-Cpf1 KIKO CAR a favorable system for rapid and efficient generation of CAR-T cells with genomic precision and modular characteristics.

### Example 5: Modular Combinations of AAV-Cpf1 Mediate Efficient Generation of CD19 and CD22 Bi-Specific CAR-T Cells with Dual TRAC;PDCD1 Disruption

### Materials and Methods

Cell culture, mRNA electroporation, and AAV vector construction and transduction were performed as previously described in Examples 1-3.

Construction of AAV Vectors

An AAV crRNA expression vector (AAV-LbcrRNA, or pXD017) containing the U6-crRNA expression cassette with crRNAs targeting the first exon of the TRAC locus and the second exon of PDCD1 was generated as described in Example 1. To generate the HDR construct, the left and right homologous arms of the TRAC or PDCD1 locus were amplified by PCR using locus-specific primer sets HDR-F1/R1 and HDR-F2/R2 from primary CD4⁺ T cells. For transgene cloning, the HDR-R1 and HDR-F2 were connected with a multiple cloning site (MCS). Homologous donor templates were cloned into the AAV-LbcrRNA with or without a crRNA. The generation of CD22BBz CAR was as previously described in Example 3. To generate CD19BBz CAR, the sequence of CD19 binding scFv (FMC63) was obtained from NCBI (GenBank: HM852952) and followed by CD8 hinge-transmembrane-regions linked to 4-1BB (CD137) intracellular domains and CD3ζ intracellular domain (Kochenderfer, J N., et al., J. Immunother., 32(7):689-702 (2009)). In order to detect CD19BBz CAR in a different way, the Flag-tag sequence (GATTACAAAGACGATGACGATAAG; (SEQ ID NO:3)) was added after the CD8a leader sequence (Han, C., et al., Nat. Commun., 9(1):468 (2018)). Based on a pXD017-dTomato backbone, the FMC63-BBz was cloned into this vector using a gBlock (IDT). For generation of the HDR template, the EFS-CAR22BBz-PA or EFS-CAR19BBz-PA cassette was cloned into the multi-clone site (MCS).

Flow Cytometry

Flow cytometry was performed as described in the previous Examples. For the CD22BBz CAR, transduced T cells were incubated with 0.2 μg CD22-Fc (R&D system) in 100 μL PBS for 30 minutes, and then stained with PE-IgG-Fc (Biolegend). For the CD19BBz CAR detection, the transduced T cells were stained with APC-anti-DYKDDDDK Tag (SEQ ID NO:11) (Biolegend). Stained cells were measured and sorted on BD FACSAria II, and analyzed using FlowJo software 9.9.4 (Treestar, Ashland, Oreg.).

Intracellular Staining of IFNγ and TNF-α

Intracellular flow cytometry was performed to detect the expression level of IFNγ and TNF-α. After infection for 4 days, AAV transduced CAR T cells were co-cultured with NALM6 in fresh media which was supplied with brefeldin A and 2 ng/mL IL-2. After being incubated for 5 hours, T cells were collected and stained for surface CAR first. After membrane protein staining, cells were fixed and permeabilized by fixation/permeabilization solution (BD), followed by addition of anti-IFNγ-APC or anti-TNF-α-FITC for intracellular staining. After 30 minutes, the stained cells were washed by BD Perm/Wash™ buffer and measured by BD FACSAria II.

Cancer Cell Killing Assay

2×10⁴ NALM6-GL cells were seeded in a 96 well plate. The modified or control T cells were co-cultured with NALM6-GL at indicated E:T ratios for 24 hours. Cell proliferation was tested by adding 150 μg/ml D-Luciferin (PerkinElmer) into each well. After 5 minutes, luciferase assay intensity was measured by a plate reader (PerkinElmer).

### Results

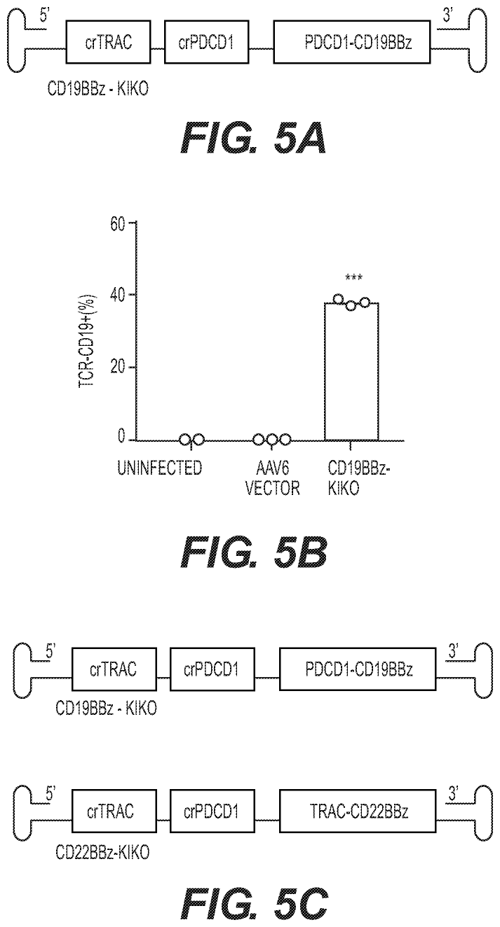

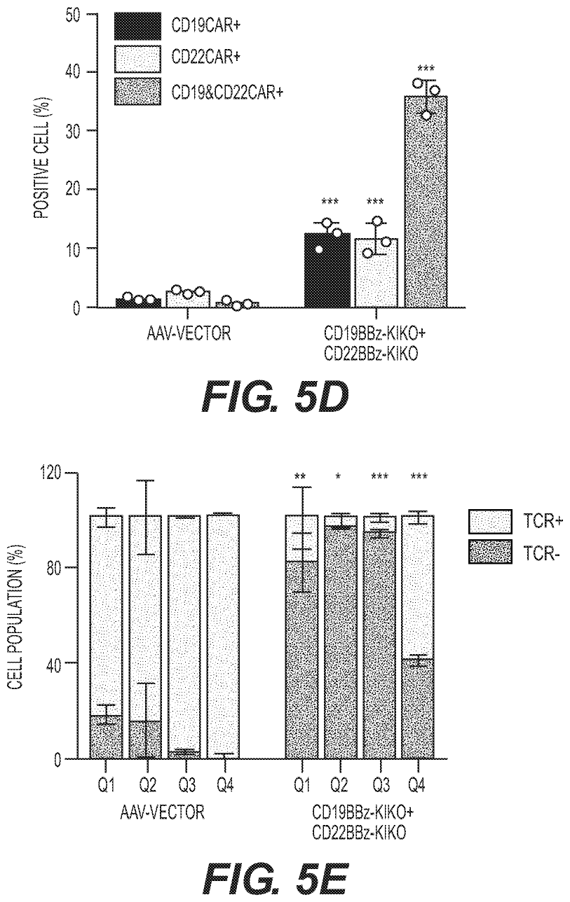

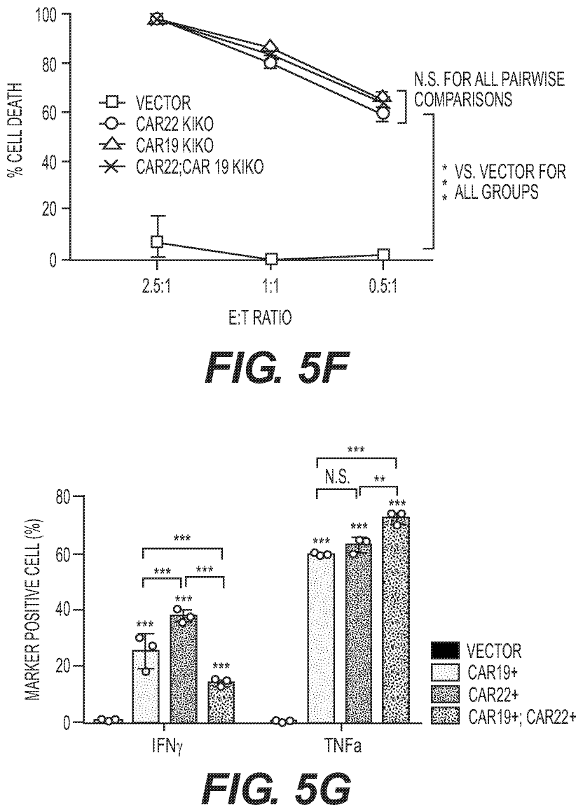

Given the results observed in Example 4, the AAV-Cpf1 KIKO system was then assessed for efficient generation of more complex CAR-Ts using simple engineering steps. First, an AAV vector designated TRAC^{KO};CD19BBz-PDCD1^{KI} (CD19BBz-KIKO for short) was generated to mediate CD19BBz transgene knock-in into the PDCD1 locus with simultaneous TRAC knockout (FIG. 5A). After LbCpf1 mRNA electroporation and AAV transduction, the efficiency of CD19BBz CAR knock-in and TCR knockout in human primary CD4+ T cells was quantified by FACS. This analysis demonstrated that one transduction generated CD19BBz-PDCD1 knock-in at a bulk efficiency of 46.87% at day 5 (not shown) and 37.83% at day 8 (FIG. 5B), with efficient TRAC knockout (FIG. 5B).

Primary CD4⁺ T cells were then jointly transduced with both CD22BBz-KIKO and CD19BBz-KIKO vectors to generate CAR-T cells that are specific to both CD22 and CD19 antigens (FIG. 5C). Five and eight days post LbCpf1 mRNA electroporation and AAV transduction, knock-in efficiencies of both CD22BBz and CD19BBz CARs were analyzed by FACS. The results revealed that one transduction generated dual knock-in of CD22BBz⁺CD19BBz⁺ double positive CAR-T cells at a bulk efficiency of 21.70% at day 5 (not shown), which further increased to 35.80% at day 8 (FIG. 5D). CD22BBz⁺ and CD19BBz⁺ single positive cells were also generated, with bulk efficiency of 22.53% and 7.27% respectively on day 5 (not shown), which were measured at 11.54% and 12.16% respectively on day 8 (FIG. 5D). The increase in the percentage of CD22BBz⁺CD19BBz⁺ double positive CAR-T cells was likely due to the negative selection of non-functional cells. Again, in all targeted T cells that underwent integration (Q1, Q2 and Q3) near-complete TCR disruption was observed, whereas vector transduced T cells mostly retained intact TCR (FIG. 5E).

These data indicate that efficient and precise double knock-ins in human T cells can be achieved by AAV-Cpf1 using either an all-in-one AAV, or two AAVs with different donors. These data demonstrated simple one-step modular generation of engineered T cells with CD19BBz and CD22BBz double CAR knock-in and simultaneous TRAC;PDCD1 dual-disruption. The phenotypes of single knock-in and double knock-in CAR-T cells generated by the AAV-Cpf1 KIKO system were then compared. Using the cognate cancer cell line NALM6, a NALM6-GL cell line that stably expressed GFP and luciferase transgenes was generated. The cytolytic activity of CAR-T cells at different titration series of effector:target (E:T) ratios in a co-culture setting (kill assay) were determined. Vector-transduced T cells showed minimal cytolytic activity against NALM6-GL. In sharp contrast, all three forms of CAR-T cells generated by Cpf1 KIKO, i.e., CAR22, CAR19 and CAR22;CAR19 double knock-ins, exhibited strong potency in killing NALM6-GL cancer cells in a dose-dependent manner (FIG. 5F). These three forms of CAR-T cells had similar cytotoxicity when compared to each other (FIG. 5F). Upon measuring the effector cytokine production, it was observed that all three forms of CAR-Ts showed highly boosted IFNγ and TNF-α production as compared to vector-transduced T cells (FIG. 5G). The CAR22;CAR19 double knock-in CAR-T cells showed relatively higher TNF-α and lower IFNγ productivity as compared to the single knock-in counterparts (FIG. 5G). These data demonstrated that both the single and double knock-in versions of the AAV-Cpf1 KIKO generated CAR-T cells are robustly functional against cognate target cancer cells.

### Example 6: AAV-Cpf1 Mediates More Efficient Generation of Double Knock-in CAR T Cells than AAV-Cas9

### Materials and Methods

Cell culture, mRNA electroporation, AAV vector construction and transduction, cell killing assay, and assaying of cytokine production and exhaustion markers were performed as previously described in Examples 1-5.

Cas9 RNP Electroporation

RNPs were produced by complexing a two-component gRNA to Cas9, as previously described (Roth, T L., et al., Nature, 559(7714):405-409 (2018)). In brief, the Cas9 guide RNA designed at the same sites with Cpf1 crRNA targeting TRAC and PDCD1 by Benchling (Table 10). crRNAs and tracrRNAs were chemically synthesized (Dharmacon, IDT), and resuspended in nuclease-free IDTE buffer at a concentration of 160 μM. The crRNA and tracrRNA were mixed at 1:1 ratio and annealed together in Nuclease-Free IDTE buffer at 95° C. for 5 min and 37° C. for 10 min (multiple guides annealed separately). RNPs were formed by the addition of SpCas9 nuclease (Dharmacon, IDT) with 80 μM gRNA (1:2 Cas9 to sgRNA molar ratio) on the benchtop for 15 min. RNPs were electroporated immediately after complexing. After 2-4 hours, AAV6 was added to cells at MOI=1e5.

TABLE 10
spCas9 guide sequences

| Gene name | Spacer Sequence (5′→3′) |
|---|---|
| hTRAC | TCTCTCAGCTGGTACACGGC (SEQ ID NO: 24) |
| hPDCD1 sg-2 | CACGAAGCTCTCCGATGTGT (SEQ ID NO: 23) |
| hPDCD1 sg-3 | CGGAGAGCTTCGTGCTAAAC (SEQ ID NO: 22) |
| hPDCD1 sg-4 | CGATGTGTTGGAGAAGCTGC (SEQ ID NO: 21) |

Flow Cytometry

Flow cytometry was performed as described in the previous Examples. In particular, for the T cell exhaustion assay, T cells from various groups were co-cultured with NALM6 cells at 0.5:1 E:T ratio for 24 hours. 1×10⁶ cells were incubated with 0.2 μg CD22-Fc (R&D Systems) in 100 μL PBS for 30 minutes and then stained with PE-IgG-Fc, PD-1-FITC, TIGIT-APC and LAG3-Percp/cy5.5 (Biolegend) for 30 minutes. After washing twice, the stained cells were measured and sorted on BD FACSAria II, and analyzed using FlowJo software 9.9.4 (Treestar, Ashland, Oreg.).

Plasmids

TABLE 11
Brief descriptions of the plasmids listed in the Examples and
throughout the text of the instant disclosure.

| Construct | | |
|---|---|---|
| name | Compositions | Targeted modifications |
| pXD017 | AAV Cpf1 crRNA | AAV backbone |
| | vector | |
| pXD017- | AAV crTRAC; | AAV with crTRAC and |
| 39 | crPDCD1 | crPDCD1 array |
| pXD039 | CD22BBz Lenti CAR | Lentivirus with EFS1α- |
| | | CD22BBz-WPRE cassette |
| pXD042 | PDCD1KO;dTomato- | AAV with crTRAC and |
| | TRACKI | crPDCD1 array and |
| | (TRAC-KIKO | HDR-EFS-dTomato- |
| | for short) | PA cassette |
| pXD043 | PDCD1KO;CD22BBz- | AAV with crTRAC and |
| | TRACKI | crPDCD1 array and HDR- |
| | (CD22BBz KIKO | EFS-CD22BBz-PA |
| | for short) | cassette |
| pXD050 | dTomato-TRACKI; | AAV with crTRAC and |
| | GFP-PDCD1KI | crPDCD1 array and HDR- |
| | (TRAC-PDCD1- | EFS-dTomato-PA&HDR- |
| | DKI for short) | EFS-GFP-PA cassette |
| pXD053 | TRACKO;GFP- | AAV with crTRAC and |
| | PDCD1KI (PDCD1- | crPDCD1 array and HDR- |
| | KIKO for short) | EFS-GFP-PA cassette |
| pXD054 | TRACKO;CD19BBz- | AAV with crTRAC and |
| | PDCD1KI | crPDCD1 array and HDR- |
| | (CD19BBz KIKO | EFS-CD19BBz- |
| | for short) | PA cassette |

### Results

The AAV-Cpf1 KIKO platform and the Cas9-mediated CAR-T generation platform were then investigated for targeting the same genes. First, the Cas9 ribonucleoprotein (RNP) with crRNA and tracrRNA (annealed together as a guide RNA) was electroporated into the cells to introduce double-stranded breaks. The electroporated cells were then infected with AAVs that carry HDR templates for CARs. Using this approach, a similar knock-in efficiency for CD22BBz CAR into the TRAC locus (CAR22) was obtained with an average of 44.73% and 53.57% CAR22⁺ T cells on days 5 and 8, respectively. This result was confirmed with two independent PDCD1 guide RNAs. Successful generation of CD19BBz CAR-T knocked into the PDCD1 locus (CAR19) was also obtained in a similar manner.

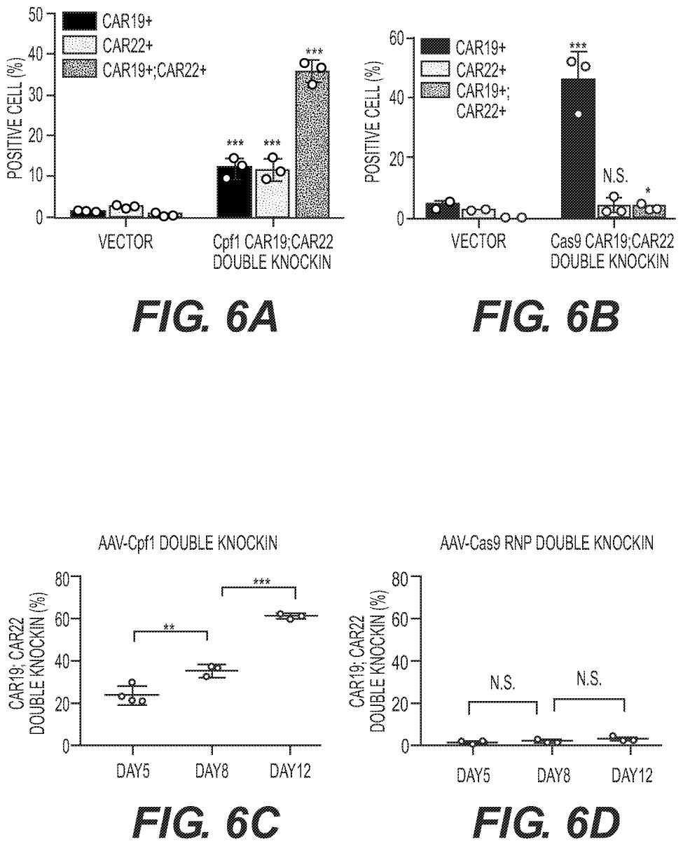

Double knock-in cells were then generated using Cas9 RNP (Cas9:crRNA:tracrRNA complex) electroporation followed by AAV infection with both CD22BBz and CD19BBz HDR templates. In parallel, double knock-in cells were generated using the AAV-Cpf1 KIKO pipeline, i.e., Cpf1 mRNA electroporation followed by AAV infection with both CD22BBz and CD19BBz HDR templates. The AAV-Cpf1 KIKO double knock-in pipeline efficiently generated CAR19⁺;CAR22⁺ double positive cells, averaging 35.80% on day 8 (FIG. 6A). The Cas9 RNP double knock-in pipeline only generated 3.41% (FIG. 6B). Using different guide RNAs did not change the efficiency of CAR19⁺;CAR22⁺ double knock-in for the Cas9 RNP system. The frequency of the CAR19±;CAR22⁺ double positive cells in the bulk unsorted population generated by the AAV-Cpf1 KIKO steadily increased from an average of 23.80% on day 5 to 61.73% on day 12 (FIG. 6C), and up to 76.30% on days 14-16. The frequency of double positive cells in the bulk unsorted population generated by the Cas9 RNP pipeline averaged at 2.56% on day 5 to 4.06% on day 12 (FIG. 6D). While Cpf1 and Cas9 represent two different nucleases and the two systems do not have strict parity, these data show that using approaches disclosed herein, the AAV-Cpf1 KIKO platform is highly efficient for generating endogenous genomic loci targeted dual knock-in CAR-Ts.

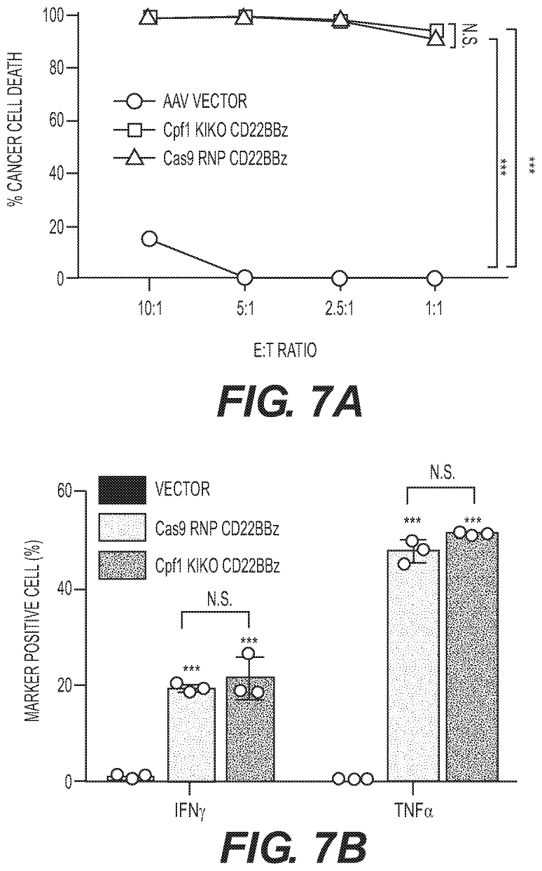

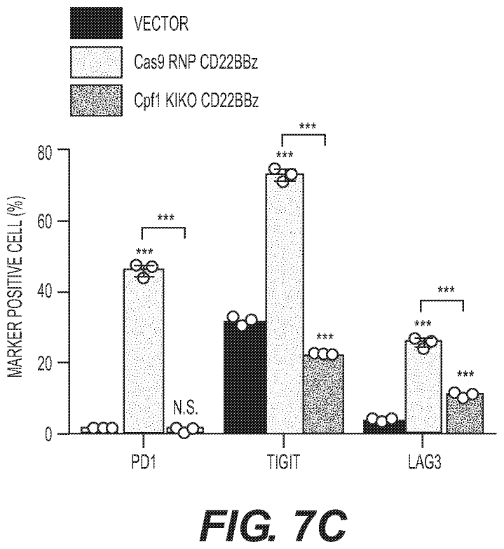

The immunological characteristics of the CAR-T cells generated by the AAV-Cpf1 KIKO and AAV-Cas9 RNP platforms were then examined in parallel. Using the previously described co-culture assay, it was observed that the CD22BBz CAR-T generated by both AAV-Cpf1 KIKO (Cpf1 KIKO CD22BBz) and AAV-Cas9 RNP (Cas9 RNP CD22BBz) were highly potent compared to vector transduced T cells, with no statistical difference between the two approaches (FIG. 7A). In addition, both Cpf1- and Cas9-generated CAR-T cells were potent IFNγ and TNF-α producers; eliciting comparable levels of IFNγ and TNF-α (FIG. 7B). In contrast to Cas9 RNP CD22BBz, the Cpf1 KIKO CD22BBz CAR-T cells expressed lower levels of T cell exhaustion markers including PD-1, TIGIT and LAGS (FIG. 7C).

In view of the efficiency data above, these experiments demonstrate that the AAV-Cpf1 KIKO CAR targeting method generates engineered CAR-T cells with potent effector function and reduced levels of exhaustion without compromising the simplicity of transgene delivery, especially when involving the generation of double knock-in CAR-Ts. These features make AAV-Cpf1 KIKO a favorable system for rapid and efficient generation of modular CAR-T cells with genomic precision and modular characteristics.

It is understood that the disclosed method and compositions are not limited to the particular methodology, protocols, and reagents described as these can vary. It is also to be understood that the terminology used herein is for the purpose of describing particular embodiments only, and is not intended to limit the scope of the present invention which will be limited only by the appended claims.

Disclosed are materials, compositions, and components that can be used for, can be used in conjunction with, can be used in preparation for, or are products of the disclosed method and compositions. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. For example, if a crRNA is disclosed and discussed and a number of modifications that can be made to a number of molecules including the crRNA are discussed, each and every combination and permutation of crRNA and the modifications that are possible are specifically contemplated unless specifically indicated to the contrary. Thus, if a class of molecules A, B, and C are disclosed as well as a class of molecules D, E, and F and an example of a combination molecule, A-D is disclosed, then even if each is not individually recited, each is individually and collectively contemplated. Thus, is this example, each of the combinations A-E, A-F, B-D, B-E, B-F, C-D, C-E, and C-F are specifically contemplated and should be considered disclosed from disclosure of A, B, and C; D, E, and F; and the example combination A-D. Likewise, any subset or combination of these is also specifically contemplated and disclosed. Thus, for example, the sub-group of A-E, B-F, and C-E are specifically contemplated and should be considered disclosed from disclosure of A, B, and C; D, E, and F; and the example combination A-D. Further, each of the materials, compositions, components, etc. contemplated and disclosed as above can also be specifically and independently included or excluded from any group, subgroup, list, set, etc. of such materials. These concepts apply to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods, and that each such combination is specifically contemplated and should be considered disclosed.

It must be noted that as used herein and in the appended claims, the singular forms “a”, “an”, and “the” include plural reference unless the context clearly dictates otherwise. Thus, for example, reference to “a crRNA” includes a plurality of such crRNAs, reference to “the crRNAs” is a reference to one or more crRNAs and equivalents thereof known to those skilled in the art, and so forth.

“Optional” or “optionally” means that the subsequently described event, circumstance, or material may or may not occur or be present, and that the description includes instances where the event, circumstance, or material occurs or is present and instances where it does not occur or is not present.

Unless the context clearly indicates otherwise, use of the word “can” indicates an option or capability of the object or condition referred to. Generally, use of “can” in this way is meant to positively state the option or capability while also leaving open that the option or capability could be absent in other forms or embodiments of the object or condition referred to. Unless the context clearly indicates otherwise, use of the word “may” indicates an option or capability of the object or condition referred to. Generally, use of “may” in this way is meant to positively state the option or capability while also leaving open that the option or capability could be absent in other forms or embodiments of the object or condition referred to. Unless the context clearly indicates otherwise, use of “may” herein does not refer to an unknown or doubtful feature of an object or condition.

Ranges can be expressed herein as from “about” one particular value, and/or to “about” another particular value. When such a range is expressed, also specifically contemplated and considered disclosed is the range from the one particular value and/or to the other particular value unless the context specifically indicates otherwise. Similarly, when values are expressed as approximations, by use of the antecedent “about,” it will be understood that the particular value forms another, specifically contemplated embodiment that should be considered disclosed unless the context specifically indicates otherwise. It will be further understood that the endpoints of each of the ranges are significant both in relation to the other endpoint, and independently of the other endpoint unless the context specifically indicates otherwise. It should be understood that all of the individual values and sub-ranges of values contained within an explicitly disclosed range are also specifically contemplated and should be considered disclosed unless the context specifically indicates otherwise. Finally, it should be understood that all ranges refer both to the recited range as a range and as a collection of individual numbers from and including the first endpoint to and including the second endpoint. In the latter case, it should be understood that any of the individual numbers can be selected as one form of the quantity, value, or feature to which the range refers. In this way, a range describes a set of numbers or values from and including the first endpoint to and including the second endpoint from which a single member of the set (i.e. a single number) can be selected as the quantity, value, or feature to which the range refers. The foregoing applies regardless of whether in particular cases some or all of these embodiments are explicitly disclosed.

Unless defined otherwise, all technical and scientific terms used herein have the same meanings as commonly understood by one of skill in the art to which the disclosed method and compositions belong. Although any methods and materials similar or equivalent to those described herein can be used in the practice or testing of the present method and compositions, the particularly useful methods, devices, and materials are as described. Publications cited herein and the material for which they are cited are hereby specifically incorporated by reference. Nothing herein is to be construed as an admission that the present invention is not entitled to antedate such disclosure by virtue of prior invention. No admission is made that any reference constitutes prior art. The discussion of references states what their authors assert, and applicants reserve the right to challenge the accuracy and pertinency of the cited documents. It will be clearly understood that, although a number of publications are referred to herein, such reference does not constitute an admission that any of these documents forms part of the common general knowledge in the art.

Although the description of materials, compositions, components, steps, techniques, etc. can include numerous options and alternatives, this should not be construed as, and is not an admission that, such options and alternatives are equivalent to each other or, in particular, are obvious alternatives. Thus, for example, a list of different gene targets does not indicate that the listed gene targets are obvious one to the other, nor is it an admission of equivalence or obviousness.

Every component disclosed herein is intended to be and should be considered to be specifically disclosed herein. Further, every subgroup that can be identified within this disclosure is intended to be and should be considered to be specifically disclosed herein. As a result, it is specifically contemplated that any component, or subgroup of components can be either specifically included for or excluded from use or included in or excluded from a list of components.

Those skilled in the art will recognize, or be able to ascertain using no more than routine experimentation, many equivalents to the specific embodiments of the method and compositions described herein. Such equivalents are intended to be encompassed by the following claims.

1. A method of modifying the genome of a cell comprising introducing to the cell an RNA-guided endonuclease, and
one or more AAV vectors at least one of which comprises a sequence that encodes one or more crRNAs, wherein the one or more crRNAs collectively direct the RNA-guided endonuclease to one or more target genes;
and optionally, wherein at least one of the AAV vectors comprises or further comprises one or more HDR templates.

2. The method of claim 1, wherein two or more of the crRNAs are encoded by a crRNA array, wherein each of the two or more crRNAs encoded by the crRNA array direct the RNA-guided endonuclease to a different target gene.

3. (canceled)

4. The method of claim 1, wherein two AAV vectors are introduced to the cell.

5. The method of claim 1, wherein at least one of the HDR templates comprises:
(a) a sequence that encodes a reporter gene, a chimeric antigen receptor (CAR), or combinations thereof; and
(b) one or more sequences collectively homologous to one or more target sites.

6. (canceled)

7. The method of claim 5, wherein the RNA-guided endonuclease induces disruption of the target genes and/or the one or more HDR templates mediate targeted integration of the reporter gene, the CAR, or a combination thereof, at the target sites.

8. The method of claim 7, wherein the target site is within the locus of the disrupted gene or at a locus different from the disrupted gene.

9. (canceled)

10. The method of claim 7, wherein the target gene or target site comprises PDCD1 or TRAC genes.

11. The method of claim 10, wherein
(a) the PDCD1 or TRAC gene is disrupted;
(b) the PDCD1 and TRAC genes are disrupted;
(c) the reporter gene, CAR, or combination thereof, is integrated in the PDCD1 or TRAC gene;
(d) the reporter genes, CARs, or combination thereof are integrated in both the PDCD1 and TRAC genes;
(e) the PDCD1 gene is disrupted and the reporter gene, CAR, or combination thereof, is integrated in the TRAC gene; or
(f) the TRAC gene is disrupted and the reporter gene, CAR, or combination thereof, is integrated in the PDCD1 gene.

12. The method of claim 5, wherein the CAR targets one or more antigens specific for cancer, an inflammatory disease, a neuronal disorder, HIV/AIDS, diabetes, a cardiovascular disease, an infectious disease, an autoimmune disease, or combinations thereof.

13. The method of claim 12, wherein the CAR is bispecific or multivalent.

14. (canceled)

15. The method of claim 12, wherein the CAR is anti-CD19 or anti-CD22.

16. (canceled)

17. The method of claim 1, wherein the RNA-guided endonuclease is provided as an mRNA that encodes the RNA-guided endonuclease, a viral vector that encodes the RNA-guided endonuclease, or an RNA-guided endonuclease protein or a complex of the RNA-guided endonuclease protein and RNA.

18-19. (canceled)

20. The method of claim 17, wherein the mRNA is introduced to the cell by electroporation, transfection, or nanoparticle mediated delivery.

21. The method of claim 1, wherein the RNA-guided endonuclease is Cpf1 or an active variant, derivative, or fragment thereof.

22-23. (canceled)

24. The method of claim 1, wherein at least one of the AAV vectors is AAV6 or AAV9.

25. The method of claim 1, wherein the introduction is performed ex vivo.

26. The method of claim 25, wherein the RNA-guided endonuclease and the one or more AAV vectors are introduced to the cell at the same or different times.

27. The method of claim 1, wherein the cell is a T cell, hematopoietic stem cell (HSC), macrophage, natural killer cell (NK), or dendritic cell (DC).

28-32. (canceled)

33. A pharmaceutical composition comprising a population of cells modified according to the method of claim 1 and a pharmaceutically acceptable buffer, carrier, diluent or excipient.

34. A method of treating a subject having a disease, disorder, or condition comprising administering to the subject an effective amount of the pharmaceutical composition of claim 33.

35. (canceled)

36. A method of treating a subject having a disease, disorder, or condition comprising administering to the subject an effective amount of a pharmaceutical composition comprising a genetically modified cell, wherein the cell is genetically modified by a method comprising introducing to the cell:
(a) an RNA-guided endonuclease; and
(b) one or more AAV vectors at least one of which comprises
(i) a sequence that encodes one or more crRNAs, wherein the one or more crRNAs collectively direct the RNA-guided endonuclease to one or more target genes; and
(ii) one or more HDR templates at least one of which comprises a sequence that encodes one or more chimeric antigen receptors (CAR); and
(iii) one or more sequences at least one of which is homologous to a target site.

37. The method of claim 36, wherein the RNA-guided endonuclease induces disruption of the one or more target genes and wherein the one or more CARs are integrated at the target site.

38. The method of claim 37, wherein the target site is within the locus of one of the disrupted genes or at a locus different from the disrupted genes.

39. (canceled)

40. The method of claim 36, wherein the target gene or target site comprises PDCD1 or TRAC genes.

41. (canceled)

42. The method of claim 36, wherein at least one of the CARs targets one or more antigens specific for or associated with the disease, disorder, or condition.

43. The method of claim 42, wherein the disease, disorder, or condition is a cancer, an inflammatory disease, a neuronal disorder, HIV/AIDS, diabetes, a cardiovascular disease, an infectious disease, or an autoimmune disease.

44. The method of claim 43, wherein the cancer is a leukemia or lymphoma selected from the group comprising chronic lymphocytic leukemia (CLL), acute lymphocytic leukemia (ALL), acute myeloid leukemia (AML), chronic myelogenous leukemia (CML), mantle cell lymphoma, non-Hodgkin's lymphoma, and Hodgkin's lymphoma.

45. (canceled)

46. The method of claim 43, wherein the at least one of the CARs targets one or more antigens selected from the group comprising AFP, AKAP-4, ALK, Androgen receptor, B7H3, BCMA, Bcr-Abl, BORIS, Carbonic, CD123, CD138, CD174, CD19, CD20, CD22, CD30, CD33, CD38, CD80, CD86, CEA, CEACAMS, CEACAM6, Cyclin, CYP1B1, EBV, EGFR, EGFR806, EGFRvIII, EpCAM, EphA2, ERG, ETV6-AML, FAP, Fos-related antigenl, Fucosyl, fusion, GD2, GD3, GloboH, GM3, gp100, GPC3, HER-2/neu, HER2, HMWMAA, HPV E6/E7, hTERT, Idiotype, IL12, IL13RA2, IM19, IX, LCK, Legumain, IgK, LMP2, MAD-CT-1, MAD-CT-2, MAGE, MelanA/MART1, Mesothelin, MET, ML-IAP, MUC1, Mutant p53, MYCN, NA17, NKG2D-L, NY-BR-1, NY-ESO-1, NY-ESO-1, OY-TES1, p53, Page4, PAP, PAX3, PAXS, PD-L1, PDGFR-β, PLAC1, Polysialic acid, Proteinase3 (PR1), PSA, PSCA, PSMA, Ras mutant, RGSS, RhoC, ROR1, SART3, sLe(a), Sperm protein 17, SSX2, STn, Survivin, Tie2, Tn, TRP-2, Tyrosinase, VEGFR2, WT1, and XAGE.

47-50. (canceled)

51. The method of claim 36, wherein the RNA-guided endonuclease is LbCpf1, or an active variant, derivative, or fragment thereof.

52. (canceled)

53. The method of claim 36, wherein the genetically modified cell is a T cell, hematopoietic stem cell (HSC), macrophage, natural killer cell (NK), or dendritic cell (DC).

54. The method of claim 53, wherein the T cell is a CD8+ T cell selected from the group consisting of effector T cells, memory T cells, central memory T cells, and effector memory T cells or a CD4+ T cell selected from the group consisting of Th1 cells, Th2 cells, Th17 cells, and Treg cells.

55-56. (canceled)

57. The method of claim 53, wherein the cell was isolated from the subject having the disease, disorder, or condition prior to the introduction to the cell.

58. The method of claim 53, wherein the cell was isolated from a healthy donor prior to the introduction to the cell.

59-61. (canceled)


SEQUENCE LISTING (29 sequences)

SEQ ID NO: 1 - DNA, 20 bp, Artificial Sequence
  TRAC guide
gagtctctcagctggtacac

SEQ ID NO: 2 - DNA, 20 bp, Artificial Sequence
  PDCD1 guide
gcacgaagctctccgatgtg

SEQ ID NO: 3 - DNA, 24 bp, Artificial Sequence
  Flag Tag sequence
gattacaaagacgatgacgataag

SEQ ID NO: 4 - DNA, 47 bp, Artificial Sequence
  TRAC_HDR_F1
tcaactagatcttgagacaaggtacgatgtaaggagctgctgtgact

SEQ ID NO: 5 - DNA, 40 bp, Artificial Sequence
  TRAC_HDR_R1
ggtacctcgagcgtacgggtcagggttctggatatctgtg

SEQ ID NO: 6 - DNA, 42 bp, Artificial Sequence
  TRAC_HDR_F2
cgtacgctcgaggtaccgagagactctaaatccagtgacaag

SEQ ID NO: 7 - DNA, 46 bp, Artificial Sequence
  TRAC_HDR_R2
cttttattaagcttgatatcgaattgtgggttaatgagtgactgcg

SEQ ID NO: 8 - DNA, 40 bp, Artificial Sequence
  PDCD1 _HDR_F1
tggcaggagagggcacgtgggcagcctcacgtagaaggaa

SEQ ID NO: 9 - DNA, 41 bp, Artificial Sequence
  PDCD1 _HDR_R1
tccgagaattctttgttaactgtgttggagaagctgcaggt

SEQ ID NO: 10 - DNA, 43 bp, Artificial Sequence
  PDCD1 _HDR_F2
cacagttaacaaagaattctcggagagcttcgtgctaaactgg

SEQ ID NO: 11 - protein, 8 aa, Artificial Sequence
  Synthetic polypeptide (DYKDDDDK tag)
DYKDDDDK

SEQ ID NO: 12 - DNA, 20 bp, Artificial Sequence
  TRAC_suvF
ctgagtcccagtccatcacg

SEQ ID NO: 13 - DNA, 19 bp, Artificial Sequence
  TRAC_suvR
agggttttggtggcaatgg

SEQ ID NO: 14 - DNA, 20 bp, Artificial Sequence
  PDCD1_suvF
gtaggtgccgctgtcattgc

SEQ ID NO: 15 - DNA, 20 bp, Artificial Sequence
  PDCD1_suvR
gagcagtgcagacaggacca

SEQ ID NO: 16 - DNA, 20 bp, Artificial Sequence
  TRAC 1st
cccttgtccatcactggcat

SEQ ID NO: 17 - DNA, 20 bp, Artificial Sequence
  TRAC 2nd
gcacacccctcatctgactt

SEQ ID NO: 18 - DNA, 19 bp, Artificial Sequence
  CD22CAR 3rd
gaaatcaaagcggccgcag

SEQ ID NO: 19 - DNA, 38 bp, Artificial Sequence
  PDCD1 _HDR_R2
gcggccgctcggtccgcacctgatcctgtgcaggaggg

SEQ ID NO: 20 - DNA, 23 bp, Artificial Sequence
  PDCD1 23nt guide
gcacgaagctctccgatgtgttg

SEQ ID NO: 21 - DNA, 20 bp, Artificial Sequence
  hPDCD1 sg-4
cgatgtgttggagaagctgc

SEQ ID NO: 22 - DNA, 20 bp, Artificial Sequence
  hPDCD1 sg-3
cggagagcttcgtgctaaac

SEQ ID NO: 23 - DNA, 20 bp, Artificial Sequence
  hPDCD1 sg-2
cacgaagctctccgatgtgt

SEQ ID NO: 24 - DNA, 20 bp, Artificial Sequence
  hTRAC
tctctcagctggtacacggc

SEQ ID NO: 25 - DNA, 50 bp, Artificial Sequence
  dTomato 3rd
aacacaggaccggttctagacgtacggccaccatggtgagcaagggcgag

SEQ ID NO: 26 - DNA, 645 bp, Artificial Sequence
  TRAC Left Arm
gatgtaaggagctgctgtgacttgctcaaggccttatatcgagtaaacggtagcgctggggcttagacgcaggtgttctgatttatagttcaaaacctctatcaatgagagagcaatctcctggtaatgtgatagatttcccaacttaatgccaacataccataaacctcccattctgctaatgcccagcctaagttggggagaccactccagattccaagatgtacagtttgctttgctgggcctttttcccatgcctgcctttactctgccagagttatattgctggggttttgaagaagatcctattaaataaaagaataagcagtattattaagtagccctgcatttcaggtttccttgagtggcaggccaggcctggccgtgaacgttcactgaaatcatggcctcttggccaagattgatagcttgtgcctgtccctgagtcccagtccatcacgagcagctggtttctaagatgctatttcccgtataaagcatgagaccgtgacttgccagccccacagagccccgcccttgtccatcactggcatctggactccagcctgggttggggcaaagagggaaatgagatcatgtcctaaccctgatcctcttgtcccacagatatccagaaccctgacc

SEQ ID NO: 27 - DNA, 659 bp, Artificial Sequence
  TRAC Right Arm
gagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagcagacaccttcttccccagcccaggtaagggcagctttggtgccttcgcaggctgtttccttgcttcaggaatggccaggttctgcccagagctctggtcaatgatgtctaaaactcctctgattggtggtctcggccttatccattgccaccaaaaccctctttttactaagaaacagtgagccttgttctggcagtccagagaatgacacggaaaaaaagcagatgaagagaaggtggcaggagagggcacgtggcccagcctcagtctctccaactgagttcctgcctgcctgcctttgctcagactgtttgccccttactgctcttctaggcctcattctaagccccttctccaagttgcctctccttatttctccctgtctgccaaaaaatctttcccagctcactaagtcagtctcacgcagtcactcattaacccac

SEQ ID NO: 28 - DNA, 304 bp, Artificial Sequence
  PDCD1 Left Arm
gcagcctcacgtagaaggaagaggctctgcagtggaggccagtgcccatccccgggtggcagaggccccagcagagacttctcaatgacattccagctggggtggcccttccagagcccttgctgcccgagggatgtgagcaggtggccggggaggctttgtggggccacccagccccttcctcacctctctccatctctcagactccccagacaggccctggaacccccccaccttctccccagccctgctcgtggtgaccgaaggggacaacgccaccttcacctgcagcttctccaacaca

SEQ ID NO: 29 - DNA, 336 bp, Artificial Sequence
  PDCD1 Right Arm
tcggagagcttcgtgctaaactggtaccgcatgagccccagcaaccagacggacaagctggccgccttccccgaggaccgcagccagcccggccaggactgccgcttccgtgtcacacaactgcccaacgggcgtgacttccacatgagcgtggtcagggcccggcgcaatgacagcggcacctacctctgtggggccatctccctggcccccaaggcgcagatcaaagagagcctgcgggcagagctcagggtgacaggtgcggcctcggaggccccggggcaggggtgagctgagccggtcctggggtgggtgtcccctcctgcacaggatcag